SEQUENCE LISTING (339 sequences)

SEQ ID NO: 1 - DNA, 960 bp, Bacillus subtilis
  CDS (1)..(960)
atgcaactattcgatctgccgctcgaccaattgcaaacatataagcctgaaaaaacagcaccgaaagatttttctgagttttggaaattgtctttggaggaacttgcaaaagtccaagcagaacctgatttacagccggttgactatcctgctgacggagtaaaagtgtaccgtctcacatataaaagcttcggaaacgcccgcattaccggatggtacgcggtgcctgacaagcaaggcccgcatccggcgatcgtgaaatatcatggctacaatgcaagctatgatggtgagattcatgaaatggtaaactgggcactccatggctacgccgcattcggcatgcttgtccgcggccagcagagcagcgaggatacgagtatttcactgcacggtcacgctttgggctggatgacgaaaggaattcttgataaagatacatactattaccgcggtgtttatttggacgccgtccgcgcgcttgaggtcatcagcagcttcgacgaggttgacgaaacaaggatcggtgtgacaggaggaagccaaggcggaggtttaaccattgccgcagcagcgctgtcagacattccaaaagccgcggttgccgattatccttatttaagcaacttcgaacgggccattgatgtggcgcttgaacagccgtaccttgaaatcaattccttcttcagaagaaatggcagcccggaaacagaagtgcaggcgatgaagacactttcatatttcgatattatgaatctcgctgaccgagtgaaggtgcctgtcctgatgtcaatcggcctgattgacaaggtcacgccgccgtccaccgtgtttgccgcctacaatcatttggaaacagagaaagagctgaaggtgtaccgctacttcggacatgagtatatccctgcttttcaaacggaaaaacttgctttctttaagcagcatcttaaaggctgataa

SEQ ID NO: 2 - protein, 318 aa, Bacillus subtilis
MQLFDLPLDQLQTYKPEKTAPKDFSEFWKLSLEELAKVQAEPDLQPVDYPADGVKVYRLTYKSFGNARITGWYAVPDKQGPHPAIVKYHGYNASYDGEIHEMVNWALHGYAAFGMLVRGQQSSEDTSISLHGHALGWMTKGILDKDTYYYRGVYLDAVRALEVISSFDEVDETRIGVTGGSQGGGLTIAAAALSDIPKAAVADYPYLSNFERAIDVALEQPYLEINSFFRRNGSPETEVQAMKTLSYFDIMNLADRVKVPVLMSIGLIDKVTPPSTVFAAYNHLETEKELKVYRYFGHEYIPAFQTEKLAFFKQHLKG

SEQ ID NO: 3 - DNA, 957 bp, Bacillus subtilis
atgcaactattcgatctgccgctcgaccaattgcaaacatataagcctgaaaaaacagcaccgaaagatttttctgagttttggaaattgtctttggaggaacttgcaaaagtccaagcagaacctgatttacagccggttgactatcctgctgacggagtaaaagtgtaccgtctcacatataaaagcttcggaaacgcccgcattaccggatggtacgcggtgcctgacaaggaaggcccgcatccggcgatcgtgaaatatcatggctacaatgcaagctatgatggtgagattcatgaaatggtaaactgggcactccatggctacgccacattcggcatgcttgtccgcggccagcagagcagcgaggatacgagtatttcaccgcacggtcacgctttgggctggatgacgaaaggaattcttgataaagatacatactattaccgcggtgtttatttggacgccgtccgcgcgcttgaggtcatcagcagcttcgacgaggttgacgaaacaaggatcggtgtgacaggaggaagccaaggcggaggtttaaccattgccgcagcagcgctgtcagacattccaaaagccgcggttgccgattatccttatttaagcaacttcgaacgggccattgatgtggcgcttgaacagccgtaccttgaaatcaattccttcttcagaagaaatggcagcccggaaacagaagtgcaggcgatgaagacactttcatatttcgatattatgaatctcgctgaccgagtgaaggtgcctgtcctgatgtcaatcggcctgattgacaaggtcacgccgccgtccaccgtgtttgccgcctacaatcatttggaaacaaagaaagagctgaaggtgtaccgctacttcggacatgagtatatccctgcttttcaaactgaaaaacttgctttctttaagcagcatcttaaaggctga

SEQ ID NO: 4 - protein, 318 aa, Bacillus subtilis
MQLFDLPLDQLQTYKPEKTAPKDFSEFWKLSLEELAKVQAEPDLQPVDYPADGVKVYRLTYKSFGNARITGWYAVPDKEGPHPAIVKYHGYNASYDGEIHEMVNWALHGYATFGMLVRGQQSSEDTSISPHGHALGWMTKGILDKDTYYYRGVYLDAVRALEVISSFDEVDETRIGVTGGSQGGGLTIAAAALSDIPKAAVADYPYLSNFERAIDVALEQPYLEINSFFRRNGSPETEVQAMKTLSYFDIMNLADRVKVPVLMSIGLIDKVTPPSTVFAAYNHLETKKELKVYRYFGHEYIPAFQTEKLAFFKQHLKG

SEQ ID NO: 5 - DNA, 957 bp, Bacillus subtilis
atgcaactattcgatctgccgctcgaccaattgcaaacgtataagcctgaaaaaacaacaccgaacgatttttctgagttttggaaatcgtctttggacgaacttgcgaaagtcaaagcagcacctgatttacagctggttgattatcctgctgatggagtcaaggtgtaccgcctcacatataaaagcttcggaaacgcccgcattaccggatggtacgcagtgcctgacaaggaaggaccgcatccggcgatcgtcaaatatcatggctacaacgctagctatgacggtgagattcatgaaatggtaaactgggcgctccacggttacgccgcattcggcatgctagtccgcggccagcagagcagcgaggatacgagtatttctccacatggccatgctttgggctggatgacgaaaggaatccttgataaagatacatactattaccggggcgtttatttggacgctgtccgcgcgcttgaggtcatcagcagctttgacgaagttgacgaaacaagaatcggtgtgacaggcggaagccaaggaggcggcttaaccattgccgcagccgctctgtcagacattccaaaagccgcggttgccgattatccttatttaagcaactttgaacgggccattgatgtggcgcttgaacagccgtaccttgaaatcaattccttctttagaagaaatggaagcccggaaacggaagagaaggcgatgaagacactttcatatttcgatattatgaatctcgctgaccgagtgaaggtccctgtcctgatgtcgatcggtctgattgacaaggtcacgccgccgtccaccgtgtttgccgcatacaaccacttggagacagagaaagagctcaaagtgtaccgctacttcgggcatgagtatatccctgcctttcaaacagaaaaacttgctttctttaagcagcatcttaaaggctga

SEQ ID NO: 6 - protein, 318 aa, Bacillus subtilis
MQLFDLPLDQLQTYKPEKTTPNDFSEFWKSSLDELAKVKAAPDLQLVDYPADGVKVYRLTYKSFGNARITGWYAVPDKEGPHPAIVKYHGYNASYDGEIHEMVNWALHGYAAFGMLVRGQQSSEDTSISPHGHALGWMTKGILDKDTYYYRGVYLDAVRALEVISSFDEVDETRIGVTGGSQGGGLTIAAAALSDIPKAAVADYPYLSNFERAIDVALEQPYLEINSFFRRNGSPETEEKAMKTLSYFDIMNLADRVKVPVLMSIGLIDKVTPPSTVFAAYNHLETEKELKVYRYFGHEYIPAFQTEKLAFFKQHLKG

SEQ ID NO: 7 - DNA, 957 bp, Bacillus licheniformis
atgcagcagccttatgatatgccgcttgaacagctttatcagtataaacctgaacggacggcaccggccgattttaaagagttctggaagggttcattggaggaattggcaaatgaaaaagcgggaccgcagcttgaaccgcatgaatatccggctgacggggtaaaagtctactggcttacatacagaagcatcgggggagcgcgaattaaaggctggtacgcagtacccgaccgccaagggcctcatcctgcgatcgtcaaataccacggctataacgcaagctatgacggagacattcacgatattgtcaattgggctcttcacggctatgcggcattcggtatgctggtccgcggacagaacagcagtgaagatacagagatctctcatcacggacatgtacccggctggatgacaaaaggaatcctcgatccgaaaacatattactacagaggggtctatttagatgccgtacgagcagtcgaagtggtcagcggttttgctgaagtcgatgaaaagcggatcggggtgatcggggcaagccaaggaggcgggctggccgtcgcggtttcggcgctgtccgatattccaaaagcagccgtgtcagaatacccttatttaagcaattttcaacgagcgatcgatacagcgatcgaccagccatatctcgaaatcaactcctttttcagaagaaacaccagtccggatattgagcaggcggccatgcataccctgtcttatttcgatgtcatgaaccttgcccaattggtcaaagcgaccgtactcatgtcgatcggactggttgacaccatcactccgccatccaccgtctttgcggcttacaatcacttggaaacggataaagaaataaaagtgtaccgttattttggacacgaatacatcccgccgttccaaaccgaaaagctggcgtttctgagaaagcatctgaaataa

SEQ ID NO: 8 - protein, 318 aa, Bacillus licheniformis
MQQPYDMPLEQLYQYKPERTAPADFKEFWKGSLEELANEKAGPQLEPHEYPADGVKVYWLTYRSIGGARIKGWYAVPDRQGPHPAIVKYHGYNASYDGDIHDIVNWALHGYAAFGMLVRGQNSSEDTEISHHGHVPGWMTKGILDPKTYYYRGVYLDAVRAVEVVSGFAEVDEKRIGVIGASQGGGLAVAVSALSDIPKAAVSEYPYLSNFQRAIDTAIDQPYLEINSFFRRNTSPDIEQAAMHTLSYFDVMNLAQLVKATVLMSIGLVDTITPPSTVFAAYNHLETDKEIKVYRYFGHEYIPPFQTEKLAFLRKHLK

SEQ ID NO: 9 - DNA, 963 bp, Bacillus pumilis
atgcaattgttcgatttatcactagaagagctaaaaaaatataaaccaaagaaaacagcacgtcctgatttctcagacttttggaagaaatcgctcgaagaactgcgccaagtggaggcagagccaacacttgaatcttatgactatccagtgaaaggcgtcaaggtgtaccgcctgacgtatcaaagctttggacattctaaaattgaaggcttttatgctgtgcctgatcaaactggtccgcatccagcgctcgttcgttttcatggctataatgccagctatgacggcggcattcacgacatcgtcaactgggcgctgcacggctatgcaacatttggtatgctcgtccgcggtcaaggtggcagtgaagacacatcagtgacaccaggcgggcatgcattagggtggatgacaaaaggcattttatcgaaagatacgtactattatcgaggcgtttatctagatgctgttcgtgcacttgaagtcattcagtctttccccgaagtagatgaacaccgtatcggcgtgatcggtggaagtcaggggggtgcgttagcgattgcggccgcagccctttcagacattccaaaagtcgttgtggcagactatccttacttatcaaattttgagcgtgcagttgatgttgccttggagcagccttatttagaaatcaattcatactttcgcagaaacagtgatccgaaagtggaggaaaaggcatttgagacattaagctattttgatttaatcaatttagctggatgggtgaaacagccaacattgatggcgatcggtctgattgacaaaataaccccaccatctactgtgtttgcggcatacaaccatttagaaacagataaagacctgaaagtatatcgctattttggacacgagtttatccctgcttttcaaacagagaagctgtcctttttacaaaagcatttgcttctatcaacataa

SEQ ID NO: 10 - protein, 320 aa, Bacillus pumilis
MQLFDLSLEELKKYKPKKTARPDFSDFWKKSLEELRQVEAEPTLESYDYPVKGVKVYRLTYQSFGHSKIEGFYAVPDQTGPHPALVRFHGYNASYDGGIHDIVNWALHGYATFGMLVRGQGGSEDTSVTPGGHALGWMTKGILSKDTYYYRGVYLDAVRALEVIQSFPEVDEHRIGVIGGSQGGALAIAAAALSDIPKVVVADYPYLSNFERAVDVALEQPYLEINSYFRRNSDPKVEEKAFETLSYFDLINLAGWVKQPTLMAIGLIDKITPPSTVFAAYNHLETDKDLKVYRYFGHEFIPAFQTEKLSFLQKHLLLST

SEQ ID NO: 11 - DNA, 963 bp, Clostridium thermocellum
atggcacaattatatgatatgcctttggaggaattaaaaaaatataagcctgcgcttacaaaacagaaagattttgatgagttttgggaaaaaagccttaaagagctggctgaaattcctttaaaatatcaacttataccttatgattttccggcccggagggtaaaagttttcagagttgaatatcttggttttaaaggtgcaaatattgaagggtggcttgccgttcccgagggagaagggttgtatcccgggcttgtacagtttcacggatacaactgggcgatggatggatgtgttcccgatgtggtaaattgggctttgaatggatatgccgcatttcttatgcttgttcggggacagcagggaagaagcgtggacaatattgtgcccggcagcggtcatgctttgggatggatgtcgaaaggtattttgtcaccggaggaatattattatagaggagtatatatggatgcggttcgtgctgttgaaattttggcttcgcttccttgtgtggatgaatcgagaataggagtgacagggggcagccagggtggaggacttgcactggcggtggctgctctgtccggcataccgaaagttgcagccgtgcattatccgtttctggcacattttgagcgtgccattgacgttgcgccggacggcccttatcttgaaattaacgaatatttaagaagaaacagcggtgaagaaatagaaagacaggtaaagaaaaccctttcctattttgatatcatgaatcttgctccccgtataaaatgccgtacttggatttgcactggtcttgtggatgagattactcctccgtcaacggtttttgcagtgtacaatcacctcaaatgcccaaaggaaatttcggtattcagatattttgggcatgaacatatgccaggaagcgttgaaatcaagctgaggatacttatggatgagctgaatccgtaa

SEQ ID NO: 12 - protein, 320 aa, Clostridium thermocellum
MAQLYDMPLEELKKYKPALTKQKDFDEFWEKSLKELAEIPLKYQLIPYDFPARRVKVFRVEYLGFKGANIEGWLAVPEGEGLYPGLVQFHGYNWAMDGCVPDVVNWALNGYAAFLMLVRGQQGRSVDNIVPGSGHALGWMSKGILSPEEYYYRGVYMDAVRAVEILASLPCVDESRIGVTGGSQGGGLALAVAALSGIPKVAAVHYPFLAHFERAIDVAPDGPYLEINEYLRRNSGEEIERQVKKTLSYFDIMNLAPRIKCRTWICTGLVDEITPPSTVFAVYNHLKCPKEISVFRYFGHEHMPGSVEIKLRILMDELNP

SEQ ID NO: 13 - DNA, 978 bp, Thermotoga neapolitana
atggccttcttcgatatgccccttgaggaactgaaaaagtaccggcctgaaaggtacgaggagaaagatttcgatgagttctggagggaaacacttaaagaaagcgaaggattccctctggatcccgtctttgaaaaggtggactttcatctcaaaacggttgaaacgtacgatgttactttctctggatacagggggcagagaataaagggctggcttcttgttccgaagttggcggaagaaaagcttccatgcgtcgtgcagtacataggttacaatggtggaaggggttttccacacgactggctgttctggccgtcaatgggttacatctgttttgtcatggacaccagggggcagggaagcggctggatgaagggagacacaccggattaccctgagggtccagtcgatccacagtaccccggattcatgacgaggggcattctggatccgggaacctattactacaggcgagtcttcgtggatgcggtcagggcggtggaagcagccatttccttcccgagagtggattccaggaaggtggtggtggccggaggcagtcagggtgggggaatcgcccttgcggtgagtgccctgtcgaacagggtgaaggctctgctctgcgatgtgccgtttctgtgccacttcagaagggccgtgcaacttgtcgacacacacccatacgtggagatcaccaacttcctcaaaacccacagggacaaagaggagattgttttcagaacactttcctacttcgatggtgtgaactttgcagcaagggcaaaggtgcccgccctgttttccgttgggctcatggacaccatctgtcctccctcgacggtcttcgccgcttacaaccactacgccggtccaaaggagatcagaatctatccgtacaacaaccacgaaggtggaggttctttccaggcaattgagcaggtgaaattcttgaagagactatttgaggaaggctag

SEQ ID NO: 14 - protein, 325 aa, Thermotoga neapolitana
MAFFDMPLEELKKYRPERYEEKDFDEFWRETLKESEGFPLDPVFEKVDFHLKTVETYDVTFSGYRGQRIKGWLLVPKLAEEKLPCVVQYIGYNGGRGFPHDWLFWPSMGYICFVMDTRGQGSGWMKGDTPDYPEGPVDPQYPGFMTRGILDPGTYYYRRVFVDAVRAVEAAISFPRVDSRKVVVAGGSQGGGIALAVSALSNRVKALLCDVPFLCHFRRAVQLVDTHPYVEITNFLKTHRDKEEIVFRTLSYFDGVNFAARAKVPALFSVGLMDTICPPSTVFAAYNHYAGPKEIRIYPYNNHEGGGSFQAIEQVKFLKRLFEEG

SEQ ID NO: 15 - DNA, 978 bp, Thermotoga maritima
atggccttcttcgatttaccactcgaagaactgaagaaatatcgtccagagcggtacgaagagaaagacttcgatgagttctgggaagagacactcgcagagagcgaaaagttccccttagaccccgtcttcgagaggatggagtctcacctcaaaacagtcgaagcgtacgatgtcaccttctccggatacaggggacagaggatcaaagggtggctccttgttccaaaactggaagaagaaaaacttccctgcgttgtgcagtacataggatacaacggtggaagaggattccctcacgactggctgttctggccttctatgggttacatatgtttcgtcatggatactcgaggtcagggaagcggctggctgaaaggagacacaccggattaccctgagggtcccgttgaccctcagtatccaggattcatgacaagaggaatactggatcccagaacttactactacagacgagtcttcacggacgctgtcagagccgttgaagctgctgcttcttttcctcaggtagatcaagaaagaatcgtgatagctggaggcagtcagggtggcggaatagcccttgcggtgagcgctctctcaaagaaagcaaaggctcttctgtgcgatgtgccgtttctgtgtcacttcagaagagcagtacagcttgtggatacgcatccatacgcggagatcacgaactttctaaagacccacagagacaaggaagaaatcgtgttcaggactctttcctatttcgatggagtgaacttcgcagccagagcgaagatccctgcgctgttttctgtgggtctcatggacaacatttgtcctccttcaacggttttcgctgcctacaattactacgctggaccgaaggaaatcagaatctatccgtacaacaaccacgagggaggaggctctttccaagcggttgaacaggtgaaattcttgaaaaaactatttgagaaaggctaa

SEQ ID NO: 16 - protein, 325 aa, Thermotoga maritima
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPALFSVGLMDNICPPSTVFAAYNYYAGPKEIRIYPYNNHEGGGSFQAVEQVKFLKKLFEKG

SEQ ID NO: 17 - DNA, 963 bp, Thermoanaerobacterium sp.
atgggacttttcgacatgccattacaaaaacttagagaatacactggtacaaatccatgccctgaagatttcgatgagtattggaatagggctttagatgagatgaggtcagttgatcctaaaattgaattgaaagaaagtagctttcaagtatcctttgcagaatgctatgacttgtactttacaggtgttcgtggtgccagaattcatgcaaagtatataaaacctaagacagaagggaaacatccagcgttgataagatttcatggatattcgtcaaattcaggcgactggaacgacaaattaaattacgtggcggcaggcttcaccgttgtggctatggatgtaagaggtcaaggagggcagtctcaagatgttggcggtgtaactgggaatactttaaatgggcatattataagagggctagacgatgatgctgataatatgcttttcaggcatattttcttagacactgcccaattggctggaatagttatgaacatgccagaagttgatgaagatagagtgggagtcatgggaccttctcaaggcggagggctgtcgttggcgtgtgctgcattggagccaagggtacgcaaagtagtatctgaatatccttttttatctgactacaagagagtttgggacttagaccttgcaaaaaacgcctatcaagagattacggactatttcaggctttttgacccaaggcatgaaagggagaatgaggtatttacaaagcttggatatatagacgttaaaaaccttgcgaaaaggataaaaggcgatgtcttaatgtgcgttgggcttatggaccaagtatgtccgccatcaactgtttttgcagcctacaacaacatacagtcaaaaaaagatataaaagtgtatcctgattatggacatgaacctatgagaggatttggagatttagcgatgcagtttatgttggaactatattcataa

SEQ ID NO: 18 - protein, 320 aa, Thermoanaerobacterium sp.
MGLFDMPLQKLREYTGTNPCPEDFDEYWNRALDEMRSVDPKIELKESSFQVSFAECYDLYFTGVRGARIHAKYIKPKTEGKHPALIRFHGYSSNSGDWNDKLNYVAAGFTVVAMDVRGQGGQSQDVGGVTGNTLNGHIIRGLDDDADNMLFRHIFLDTAQLAGIVMNMPEVDEDRVGVMGPSQGGGLSLACAALEPRVRKVVSEYPFLSDYKRVWDLDLAKNAYQEITDYFRLFDPRHERENEVFTKLGYIDVKNLAKRIKGDVLMCVGLMDQVCPPSTVFAAYNNIQSKKDIKVYPDYGHEPMRGFGDLAMQFMLELYS

SEQ ID NO: 19 - DNA, 978 bp, Bacillus sp.
  CDS (1)..(978)
atgaacctttttgatatgccccttgaggagctgcagcattacaagcctgcccagaccaggcaggatgattttgagtcattctggaaaaagcggattgaggagaacagtcaatatccgctgaatatagaagtaatggagcgggtttatccggttccgggagtgagagtatatgatatttattttgacgggttccggaattcccgcatccatggggtgtatgttactccagaaactccgggagcggacactcctgcggcagtgatttttcacggctataactggaacacgctgcagccgcattacagcttcaagcacgtgattcaggggattcctgtactgatggtggaggtgcggggacaaaatctcttgtctccagatagaaatcattatgggaatggaggtccgggaggctggatgacactcggcgtgatggatcccgatcaatattattacagcctggtatatatggactgcttccgcagcattgatgctgtcagggaactgtcgaggaagagaagtgtgtttgtggaaggcggaagccagggaggtgcactggcgattgccgcagccgccctgcaggatgacatcctgcttgcactcgccgacatcccttttctcacccatttcaagcgttccgtggagctttcctcggatggaccgtatcaggagatttcccactacttcaaagttcatgatcctcttcatcaaacggaagagcaggtatatcagacgctcagctatgtggactgcatgaacatggccagcatggttgaatgtccagtccttctttcagccggtctggaagacatcgtttgtcccccgtccagtgcatttgcactgttcaaccatctcggcgggccaaaagaaatacgggcctatccggaatacgcccatgaagtaccggctgtccatgaagaggaaaagctgaagtttatatcttcaaggctaaaaaatagagaaaagaggtgccggccatga

SEQ ID NO: 20 - protein, 325 aa, Bacillus sp.
MNLFDMPLEELQHYKPAQTRQDDFESFWKKRIEENSQYPLNIEVMERVYPVPGVRVYDIYFDGFRNSRIHGVYVTPETPGADTPAAVIFHGYNWNTLQPHYSFKHVIQGIPVLMVEVRGQNLLSPDRNHYGNGGPGGWMTLGVMDPDQYYYSLVYMDCFRSIDAVRELSRKRSVFVEGGSQGGALAIAAAALQDDILLALADIPFLTHFKRSVELSSDGPYQEISHYFKVHDPLHQTEEQVYQTLSYVDCMNMASMVECPVLLSAGLEDIVCPPSSAFALFNHLGGPKEIRAYPEYAHEVPAVHEEEKLKFISSRLKNREKRCRP

SEQ ID NO: 21 - DNA, 960 bp, Bacillus halodurans
ttagagatcagataaaaattgaaaaatccgatcacgatggcctggcaaatcttcgtgagcaaagtctggatataactcgatactttttgtcgtcgtgagtttgttatacatggcaaattgtgtagacggcgggcaaaccgtatccattaacccaacagcaagtaagacttctccctttacgagtggagcaagatgctgaatatcaatatagcctagcttcgtaaagatttcagcctcacgtcggtgctgtggatcaaagcgacgaaaatacgtttgcaattcgtcataagctttctcggctaaatccatctcccatacgcgttggtaatcgctaaggaaaggataaacaggagctacctttttaattttcggttccaaagccgcacaagcaatcgctaaggcccctccttgtgaccaacctgtcactgccacgcgctcttcatcgacttcaggaaggttcatcacaatgttggcaagctgagccgtatcaagaaacacatgacggaacaataattgatcagcattatcatcgagtccgcgtattatatgaccggaatgagtattccccttcacgcctcctgtgtcttcagacaagcctccttgcccgcgaacgtccattgcaagaacagaatatccgagggctgcgtaatgaagtaaacccgtccattcccccgcattcatcgtatatccgtgaaaatgaataaccgccgggtgtgtcccgctcgtgtgtcttgggcgcacgtattttgcgtgaattctagcacccctaacccctgtaaaatataggtggaagcattctgcatacgtggtttgaaaatcactcggtatgagctctacgtttggatttacctttctcatctcttgtaaagcacgatcccaatactcagtaaagtcatctggctttggattacgtcccatgtactcttttaattcggttaacggcatgtctattagtggcat

SEQ ID NO: 22 - protein, 319 aa, Bacillus halodurans
MPLIDMPLTELKEYMGRNPKPDDFTEYWDRALQEMRKVNPNVELIPSDFQTTYAECFHLYFTGVRGARIHAKYVRPRHTSGTHPAVIHFHGYTMNAGEWTGLLHYAALGYSVLAMDVRGQGGLSEDTGGVKGNTHSGHIIRGLDDNADQLLFRHVFLDTAQLANIVMNLPEVDEERVAVTGWSQGGALAIACAALEPKIKKVAPVYPFLSDYQRVWEMDLAEKAYDELQTYFRRFDPQHRREAEIFTKLGYIDIQHLAPLVKGEVLLAVGLMDTVCPPSTQFAMYNKLTTTKSIELYPDFAHEDLPGHRDRIFQFLSDL

SEQ ID NO: 23 - DNA, 954 bp, Bacillus clausii
atgccattagtcgatatgccgttgcgcgagttgttagcttatgaaggaataaaccctaaaccagcagattttgaccaatactggaaccgggccaaaacggaaattgaagcgattgatcccgaagtcactctagtcgaatcttctttccagtgttcgtttgcaaactgttaccatttctattatcgaagcgctggaaatgcaaaaatccatgcgaaatacgtacagccaaaagcaggggagaagacgccagcagtttttatgttccatgggtatggggggcgttcagccgaatggagcagcttgttaaattatgtagcggcgggtttttctgttttctatatggacgtgcgtggacaaggtggaacttcagaggatcctgggggcgtaagggggaatacatataggggccacattattcgcggcctcgatgccgggccagacgcacttttttaccgcagcgttttcttggacaccgtccaattggttcgtgctgctaaaacattgcctcacatcgataaaacacggcttatggccacagggtggtcgcaagggggcgccttaacgcttgcctgtgctgcccttgttcctgaaatcaagcgtcttgctccagtatacccgtttttaagcgattacaagcgagtgtggcaaatggatttagcggttcgttcgtataaagaattggctgattatttccgttcatacgatccgcaacataaacgccatggcgaaatttttgaacgccttggctacatcgatgtccagcatcttgctgaccggattcaaggagatgtcctaatgggagttggtttaatggatacagaatgcccgccgtctacccaatttgctgcttataataaaataaaggctaaaaaatcgtatgagctctatcctgattttggccatgagcaccttccaggaatgaacgatcatatttttcgctttttcactagttga

SEQ ID NO: 24 - protein, 317 aa, Bacillus clausii
MPLVDMPLRELLAYEGINPKPADFDQYWNRAKTEIEAIDPEVTLVESSFQCSFANCYHFYYRSAGNAKIHAKYVQPKAGEKTPAVFMFHGYGGRSAEWSSLLNYVAAGFSVFYMDVRGQGGTSEDPGGVRGNTYRGHIIRGLDAGPDALFYRSVFLDTVQLVRAAKTLPHIDKTRLMATGWSQGGALTLACAALVPEIKRLAPVYPFLSDYKRVWQMDLAVRSYKELADYFRSYDPQHKRHGEIFERLGYIDVQHLADRIQGDVLMGVGLMDTECPPSTQFAAYNKIKAKKSYELYPDFGHEHLPGMNDHIFRFFTS

SEQ ID NO: 25 - DNA, 960 bp, Bacillus subtilis
  CDS (1)..(960)
atgcaactattcgatctgccgctcgaccaattgcaaacatataagcctgaaaaaacagcaccgaaagatttttctgagttttggaaattgtctttggaggaacttgcaaaagtccaagcagaacctgatctacagccggttgactatcctgctgacggagtaaaagtgtaccgtctcacatataaaagcttcggaaacgcccgcattaccggatggtacgcggtgcctgacaagcaaggcccgcatccggcgatcgtgaaatatcatggctacaatgcaagctatgatggtgagattcatgaaatggtaaactgggcactccatggctacgccgcattcggcatgcttgtccgcggccagcagagcagcgaggatacgagtatttcaccgcacggtcacgctttgggctggatgacgaaaggaattcttgataaagatacatactattaccgcggtgtttatttggacgccgtccgcgcgcttgaggtcatcagcagcttcgacgaggttgacgaaacaaggatcggtgtgacaggaggaagccaaggcggaggtttaaccattgccgcagcagcgctgtcagacattccaaaagccgcggttgccgattatccttatttaagcaacttcgaacgggccattgatgtggcgcttgaacagccgtaccttgaaatcaattccttcttcagaagaaatggcagcccggaaacagaagtgcaggcgatgaagacactttcatatttcgatattatgaatctcgctgaccgagtgaaggtgcctgtcctgatgtcaatcggcctgattgacaaggtcacgccgccatccaccgtgtttgccgcctacaatcatttggaaacagagaaagagctgaaggtgtaccgctacttcggacatgagtatatccctgcttttcaaacggaaaaacttgctttctttaagcagcatcttaaaggctgataa

SEQ ID NO: 26 - protein, 318 aa, Bacillus subtilis
MQLFDLPLDQLQTYKPEKTAPKDFSEFWKLSLEELAKVQAEPDLQPVDYPADGVKVYRLTYKSFGNARITGWYAVPDKQGPHPAIVKYHGYNASYDGEIHEMVNWALHGYAAFGMLVRGQQSSEDTSISPHGHALGWMTKGILDKDTYYYRGVYLDAVRALEVISSFDEVDETRIGVTGGSQGGGLTIAAAALSDIPKAAVADYPYLSNFERAIDVALEQPYLEINSFFRRNGSPETEVQAMKTLSYFDIMNLADRVKVPVLMSIGLIDKVTPPSTVFAAYNHLETEKELKVYRYFGHEYIPAFQTEKLAFFKQHLKG

SEQ ID NO: 27 - protein, 325 aa, Thermotoga neapolitana
  MISC_FEATURE (277)..(277): Xaa is Ala, Val, Ser, or Thr.
MAFFDMPLEELKKYRPERYEEKDFDEFWRETLKESEGFPLDPVFEKVDFHLKTVETYDVTFSGYRGQRIKGWLLVPKLAEEKLPCVVQYIGYNGGRGFPHDWLFWPSMGYICFVMDTRGQGSGWMKGDTPDYPEGPVDPQYPGFMTRGILDPGTYYYRRVFVDAVRAVEAAISFPRVDSRKVVVAGGSQGGGIALAVSALSNRVKALLCDVPFLCHFRRAVQLVDTHPYVEITNFLKTHRDKEEIVFRTLSYFDGVNFAARAKVPALFSVGLMDTIXPPSTVFAAYNHYAGPKEIRIYPYNNHEGGGSFQAIEQVKFLKRLFEEG

SEQ ID NO: 28 - protein, 325 aa, Thermotoga maritima
  MISC_FEATURE (277)..(277): Xaa is Ala, Val, Ser, or Thr.
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPALFSVGLMDNIXPPSTVFAAYNYYAGPKEIRIYPYNNHEGGGSFQAVEQVKFLKKLFEKG

SEQ ID NO: 29 - protein, 326 aa, Thermotoga lettingae
  MISC_FEATURE (277)..(277): Xaa is Ala, Val, Ser, or Thr.
MVYFDMPLEDLRKYLPQRYEEKDFDDFWKQTIHETRGYFQEPILKKVDFYLQNVETFDVTFSGYRGQKIKGWLILPKFRNGKLPCVVEFVGYGGGRGFPYDWLLWSAAGYAHFIMDTRGQGSNWMKGDTPDYEDNPSDPQYPGFLTKGVLNPETYYYRRVFMDAFMAVETISQLEQIDSQTIILSGASQGGGIALAVSALSSKVMALLCDVPFLCHYKRAVQITDSMPYAEITRYCKTHIDKIQTVFRTLSYFDGVNFAARAKCPALFSVGLMDDIXPPSTVFAAYNYYAGEKDIRIYPYNNHEGGGSFHTLEKLKFVKKTISMRE

SEQ ID NO: 30 - protein, 325 aa, Thermotoga petrophilia
  MISC_FEATURE (277)..(277): Xaa is Ala, Val, Ser, or Thr.
MAFFDLPLEELKKYRPERYEEKDFDEFWEGTLAENEKFPLDPVFERMESHLKTVEAYDVTFSGYMGQRIKGWLLVPKLEEEKLPCVVQYIGYNGGRGFPHDWLFWPSMGYICFVMDTRGQGSGWMKGDTPDYPEDPVDPQYPGFMTRGILDPRTYYYRRVFTDAVRAVEAAASFPRVDHERIVIAGGSQGGGIALAVSALSKKAKALLCDVPFLCHFRRAVQLVDTHPYAEITNFLKTHRDKEEIVFRTLSYFDGVNFAVRAKIPALFSVGLMDNIXPPSTVFAAYNHYAGPKEIRIYPYNNHEGGGSFQAIEQVKFLKRLFEKG

SEQ ID NO: 31 - protein, 325 aa, Thermotoga sp. RQ2a
  MISC_FEATURE (277)..(277): Xaa is Ala, Val, Ser, or Thr.
MAFFDLPLEELKKYRPERYEEKDFDEFWKETLAESEKFPLDPVFERMESHLKTVEVYDVTFSGYRGQRIKGWLLVPKLEEEKLPCVVQYIGYNGGRGFPHDWLFWPSMGYICFVMDTRGQGSGWLKGDTPDYPEDPVDPQYPGFMTRGILDPRTYYYRRVFTDAVRAVEAAASFPRVDHERIVIAGGSQGGGIALAVSALSKKAKALLCDVPFLCHFRRAVQLVDTHPYAEITNFLKTHRDKEEIVFRTLSYFDGVNFAVRAKIPALFSVGLMDNIXPPSTVFAAYNHYAGPKEIRIYPYNNHEGGGSFQAIEQVKFLKRLFEKG

SEQ ID NO: 32 - protein, 329 aa, Thermotoga sp. RQ2b
  MISC_FEATURE (278)..(278): Xaa is Ala, Val, Ser, or Thr.
MALFDMPLEKLRSYLPDRYEEEDFDLFWKETLEESRKFPLDPIFERVDYLLENVEVYDVTFSGYRGQRIKAWLILPVVKKEERLPCIVEFIGYRGGRGFPFDWLFWSSAGYAHFVMDTRGQGTSRVKGDTPDYCDEPINPQFPGFMTRGILDPRTYYYRRVFTDAVRAVETASSFPGIDPERIAVVGTSQGGGIALAVAALSEIPKALVSNVPFLCHFRRAVQITDNAPYSEIVNYLKVHRDKEEIVFRTLSYFDGVNFAARAKIPALFSVALMDKTXPPSTVFAAYNHYAGPKEIKVYPFNEHEGGESFQRMEELRFMKRILKGEFKA

SEQ ID NO: 33 - protein, 326 aa, Thermotoga lettingae
MVYFDMPLEDLRKYLPQRYEEKDFDDFWKQTIHETRGYFQEPILKKVDFYLQNVETFDVTFSGYRGQKIKGWLILPKFRNGKLPCVVEFVGYGGGRGFPYDWLLWSAAGYAHFIMDTRGQGSNWMKGDTPDYEDNPSDPQYPGFLTKGVLNPETYYYRRVFMDAFMAVETISQLEQIDSQTIILSGASQGGGIALAVSALSSKVMALLCDVPFLCHYKRAVQITDSMPYAEITRYCKTHIDKIQTVFRTLSYFDGVNFAARAKCPALFSVGLMDDICPPSTVFAAYNYYAGEKDIRIYPYNNHEGGGSFHTLEKLKFVKKTISMRE

SEQ ID NO: 34 - protein, 325 aa, Thermotoga petrophilia
MAFFDLPLEELKKYRPERYEEKDFDEFWEGTLAENEKFPLDPVFERMESHLKTVEAYDVTFSGYMGQRIKGWLLVPKLEEEKLPCVVQYIGYNGGRGFPHDWLFWPSMGYICFVMDTRGQGSGWMKGDTPDYPEDPVDPQYPGFMTRGILDPRTYYYRRVFTDAVRAVEAAASFPRVDHERIVIAGGSQGGGIALAVSALSKKAKALLCDVPFLCHFRRAVQLVDTHPYAEITNFLKTHRDKEEIVFRTLSYFDGVNFAVRAKIPALFSVGLMDNICPPSTVFAAYNHYAGPKEIRIYPYNNHEGGGSFQAIEQVKFLKRLFEKG

SEQ ID NO: 35 - protein, 325 aa, Thermotoga sp. RQ2a
MAFFDLPLEELKKYRPERYEEKDFDEFWKETLAESEKFPLDPVFERMESHLKTVEVYDVTFSGYRGQRIKGWLLVPKLEEEKLPCVVQYIGYNGGRGFPHDWLFWPSMGYICFVMDTRGQGSGWLKGDTPDYPEDPVDPQYPGFMTRGILDPRTYYYRRVFTDAVRAVEAAASFPRVDHERIVIAGGSQGGGIALAVSALSKKAKALLCDVPFLCHFRRAVQLVDTHPYAEITNFLKTHRDKEEIVFRTLSYFDGVNFAVRAKIPALFSVGLMDNICPPSTVFAAYNHYAGPKEIRIYPYNNHEGGGSFQAIEQVKFLKRLFEKG

SEQ ID NO: 36 - protein, 329 aa, Thermotoga sp. RQ2b
MALFDMPLEKLRSYLPDRYEEEDFDLFWKETLEESRKFPLDPIFERVDYLLENVEVYDVTFSGYRGQRIKAWLILPVVKKEERLPCIVEFIGYRGGRGFPFDWLFWSSAGYAHFVMDTRGQGTSRVKGDTPDYCDEPINPQFPGFMTRGILDPRTYYYRRVFTDAVRAVETASSFPGIDPERIAVVGTSQGGGIALAVAALSEIPKALVSNVPFLCHFRRAVQITDNAPYSEIVNYLKVHRDKEEIVFRTLSYFDGVNFAARAKIPALFSVALMDKTCPPSTVFAAYNHYAGPKEIKVYPFNEHEGGESFQRMEELRFMKRILKGEFKA

SEQ ID NO: 37 - DNA, 960 bp, Thermoanaerobacterium saccharolyticum
atgggtctgttcgatatgccactgcaaaaactgcgtgaatataccggtaccaacccatgtcctgaggatttcgatgaatactgggatcgcgcactggacgaaatgcgtagcgttgatcctaaaatcaagatgaagaagagctcctttcaagttccgttcgcggaatgttacgatctgtattttaccggcgttcgtggtgcccgcattcacgcgaaatacattcgtccgaaaaccgaaggcaaacacccggcgctgattcgcttccatggttactccagcaactctggtgattggaacgacaagctgaactacgttgcggctggttttaccgtagtagcgatggacgctcgtggccagggtggccaatctcaggacgtcggcggtgttaatggcaacaccctgaacggtcacatcatccgtggcctggacgatgatgcagataacatgctgttccgtcatattttcctggacaccgcgcagctggctggtatcgttatgaacatgccggaaatcgatgaggaccgcgtagctgttatgggtccgtcccagggcggcggtctgtccctggcgtgtgcggctctggaacctaaaatccgtaaagtagtgtccgaatatccgttcctgagcgactacaagcgtgtgtgggatctggatctggccaaaaatgcgtaccaagaaatcactgactatttccgtctgttcgacccacgccacgaacgtgagaacgaggtttttactaaactgggttacattgacgtaaagaacctggcgaaacgtatcaaaggtgatgttctgatgtgcgtgggcctgatggatcaggtctgcccgccgagcaccgtatttgcagcatacaacaacatccagtccaagaaggacatcaaagtctacccggactatggtcacgaaccgatgcgtggcttcggtgacctggctatgcagttcatgctggaactgtattct

SEQ ID NO: 38 - protein, 320 aa, Thermoanaerobacterium saccharolyticum
MGLFDMPLQKLREYTGTNPCPEDFDEYWDRALDEMRSVDPKIKMKKSSFQVPFAECYDLYFTGVRGARIHAKYIRPKTEGKHPALIRFHGYSSNSGDWNDKLNYVAAGFTVVAMDARGQGGQSQDVGGVNGNTLNGHIIRGLDDDADNMLFRHIFLDTAQLAGIVMNMPEIDEDRVAVMGPSQGGGLSLACAALEPKIRKVVSEYPFLSDYKRVWDLDLAKNAYQEITDYFRLFDPRHERENEVFTKLGYIDVKNLAKRIKGDVLMCVGLMDQVCPPSTVFAAYNNIQSKKDIKVYPDYGHEPMRGFGDLAMQFMLELYS

SEQ ID NO: 39 - DNA, 939 bp, Lactococcus lactis
atgacaaaaataaacaattggcaagattatcaaggaagttcacttaaaccagaggattttgataaattttgggatgaaaaaattaatttggtttcaaatcatcaatttgaatttgaattaatagaaaaaaatctttcctctaaggtagttaacttttatcatttgtggtttacagctattgatggagctaaaattcatgctcagttaattgttcccaagaatttgaaagagaaatacccagccatcttacaatttcatggttatcattgcgatagtggggattgggtcgataaaatagggatagttgccgaagggaatgtagttcttgcgcttgattgtcgaggacaaggtggtttaagtcaagataatattcaaactatggggatgacaatgaagggactcattgttcgaggaattgatgaagggtatgaaaatctctattacgttcgccaatttatggacttaataactgcaaccaaaattttatccgagtttgattttgttgatgaaacaaatataagtgcacaaggtgcttctcaaggtggagcgcttgccgttgcttgcgccgcactttctcctcttataaaaaaggtgactgccacttacccctttctttcagattatcgcaaagcttatgagcttggtgccgaggaatctgctttcgaagaacttccatattggtttcagtttaaagatccacttcatctaagagaagactggttttttaatcagttggaatacattgatattcaaaatttagcaccaagaattaaggctgaggtcatttggatcctaggcggcaaagatactgttgttcctccgattacgcaaatggcggcttacaataaaatacaaagtaaaaaatctctctatgtcttacctgaatacggccatgaatatcttcctaaaattagcgactggttaagagagaatcaataa

SEQ ID NO: 40 - protein, 312 aa, Lactococcus lactis
MTKINNWQDYQGSSLKPEDFDKFWDEKINLVSNHQFEFELIEKNLSSKVVNFYHLWFTAIDGAKIHAQLIVPKNLKEKYPAILQFHGYHCDSGDWVDKIGIVAEGNVVLALDCRGQGGLSQDNIQTMGMTMKGLIVRGIDEGYENLYYVRQFMDLITATKILSEFDFVDETNISAQGASQGGALAVACAALSPLIKKVTATYPFLSDYRKAYELGAEESAFEELPYWFQFKDPLHLREDWFFNQLEYIDIQNLAPRIKAEVIWILGGKDTVVPPITQMAAYNKIQSKKSLYVLPEYGHEYLPKISDWLRENQ

SEQ ID NO: 41 - DNA, 972 bp, Mesorhizobium loti
atgccgttcccggatctgatccagcccgaactgggcgcttatgtcagcagtgtcggcatgccggacgactttgcccaattctggacgtcgaccatcgccgaggctcgccaggccggcggtgaggtcagtatcgtgcaggcgcagacgacactgaaggcggtccagtccttcgatgtcacgtttccaggatacggcggtcatccaatcaaaggatggctgatcttgccgacgcaccacaaggggcggcttcccctcgtcgtgcagtatatcggctatggcggcggccgcggcttggcgcatgagcaactgcattgggcggcgtcaggctttgcctatttccgaatggatacacgcgggcagggaagcgactggagcgtcggtgagaccgccgatcccgtcggctcgacctcgtccattcccggctttatgacgcgtggcgtgctggacaagaatgactactattaccggcgcctgttcaccgatgccgtgagggcgatagatgctctgctcggactggacttcgtcgatcccgaacgcatcgcggtttgcggtgacagtcagggaggcggtatttcgctcgccgttggcggcatcgacccgcgcgtcaaggccgtaatgcccgacgttccatttctgtgcgactttccgcgcgctgtgcagactgccgtgcgcgatccctatttggaaatcgttcgctttctggcccagcatcgcgaaaagaaggcggcagtctttgaaacgctcaactatttcgactgcgtcaacttcgcccggcggtccaaggcgccggcgctgttttcggtggccctgatggacgaagtctgcccgccctctaccgtgtatggcgcattcaatgcctatgcaggcgaaaagaccatcacagagtacgaattcaacaatcatgaaggcgggcaaggctatcaagagcgccaacagatgacgtggctcagcaggctgttcggtgtcggctga

SEQ ID NO: 42 - protein, 323 aa, Mesorhizobium loit
MPFPDLIQPELGAYVSSVGMPDDFAQFWTSTIAEARQAGGEVSIVQAQTTLKAVQSFDVTFPGYGGHPIKGWLILPTHHKGRLPLVVQYIGYGGGRGLAHEQLHWAASGFAYFRMDTRGQGSDWSVGETADPVGSTSSIPGFMTRGVLDKNDYYYRRLFTDAVRAIDALLGLDFVDPERIAVCGDSQGGGISLAVGGIDPRVKAVMPDVPFLCDFPRAVQTAVRDPYLEIVRFLAQHREKKAAVFETLNYFDCVNFARRSKAPALFSVALMDEVCPPSTVYGAFNAYAGEKTITEYEFNNHEGGQGYQERQQMTWLSRLFGVG

SEQ ID NO: 43 - DNA, 990 bp, Geobacillus stearothermophilus
atgttcgatatgccgttagcacaattacagaaatacatggggacaaatccgaagccggctgattttgctgacttttggagtcgagcgttggaggaattatctgcccaatcgttgcattatgagctgattccggcaacatttcaaacgacagtggcgagttgctaccatttgtatttcacgggagtcggcggggctagagtccattgtcagttagtaaaaccgagagagcagaagcagaaaggcccggggttggtatggtttcatggctaccatacgaatagcggcgattgggtcgataaactggcatatgctgcggcaggttttactgtattggcgatggattgccgcggccaaggaggaaaatcagaggataatttgcaagtgaaaggcccaacattgaagggccatattattcgcggaattgaggatccaaatcctcatcatctttattatcgaaatgtttttttagatacagttcaggcggtaagaattttatgctctatggatcatattgatcgtgaacgaattggtgtatatggcgcttcccaaggaggagcgttggcattagcgtgtgctgctctggaaccatcggtggtgaaaaaagcggttgtgctctatccatttttatcggattataagcgggcgcaagagttggatatgaaaaataccgcgtatgaggaaattcattattattttcgatttttagatcccacacatgagcgggaagaagaagtattttacaaactaggctatattgatattcaactcttagccgatcggatttgtgccgatgttttatgggctgttgcgctagaagaccatatttgtcccccgtccacacaatttgctgtttataataaaattaagtcaaaaaaagacatggttttgttttacgagtatggtcatgagtatttaccgactatgggagaccgtgcttatctgtttttttgcccgatcttctttccaatccaaaagagaaacgttaagtaa

SEQ ID NO: 44 - protein, 329 aa, Geobacillus stearothermophilus
MFDMPLAQLQKYMGTNPKPADFADFWSRALEELSAQSLHYELIPATFQTTVASCYHLYFTGVGGARVHCQLVKPREQKQKGPGLVWFHGYHTNSGDWVDKLAYAAAGFTVLAMDCRGQGGKSEDNLQVKGPTLKGHIIRGIEDPNPHHLYYRNVFLDTVQAVRILCSMDHIDRERIGVYGASQGGALALACAALEPSVVKKAVVLYPFLSDYKRAQELDMKNTAYEEIHYYFRFLDPTHEREEEVFYKLGYIDIQLLADRICADVLWAVALEDHICPPSTQFAVYNKIKSKKDMVLFYEYGHEYLPTMGDRAYLFFCPIFFPIQKRNVK

SEQ ID NO: 45 - DNA, 978 bp, artificial sequence
  synthetic construct
atggcgttcttcgacctgcctctggaagaactgaagaaataccgtccagagcgttacgaagagaaggacatcgacgagttctgggaggaaactctggcggagaccgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggctacatttgcttcgtcatggatactcgtggtcagggttccggctggctgaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctcaggttgacctggagcgtattgttatcgctggtggctcccagggtggcggcatcgccctggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttttccgcaccctgtcctatttcgacggcgttaacttcgcggctcgtgcaaaaattccggcactgttctctgttggtctgatggacgacatcagccctccttctaccgttttcgcggcatataactattatgcgggtccgaaagaaatccgtatctatccgtacaacaaccacgaaggcggtggtggctttcaggctgttgaacaagtgaaatccctgaagaaactgtttgagaagggctaa

SEQ ID NO: 46 - protein, 325 aa, artificial sequence
  synthetic construct
MAFFDLPLEELKKYRPERYEEKDIDEFWEETLAETEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDLERIVIAGGSQGGGIALAVSALSKKAKALLCDVPFLCHFRRAVQLVDTHPYAEITNFLKTHRDKEEIVFRTLSYFDGVNFAARAKIPALFSVGLMDDISPPSTVFAAYNYYAGPKEIRIYPYNNHEGGGGFQAVEQVKSLKKLFEKG

SEQ ID NO: 47 - DNA, 978 bp, artificial sequence
  synthetic construct
atggcgttcttcgacctgcctctggaagaactgaagaaataccgtccagagcgttacgaagagaaggacttcgacgagttctgggaggaaactctggcggagagcgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggctacatttgcttcgtcatggatactcgtggtcagggttccggctggctgaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctcaggttgaccaggagcgtattgttatcgctggtggctcccagggtggcggcatcgccctggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttttccgcaccctgtcctatttcgacggcgttaacttcgcggctcgtgcaaaaattccggcactgttctctgttggtctgatggacgacatcagccctccttctaccgttttcgcggcatataactattatgcgggtccgaaagaaatccgtatctatccgtacaacaaccacgaaggcggtggtagctttcaggctgttgaacaagtgaaattcctgaagaaactgtttgagaagggctaa

SEQ ID NO: 48 - protein, 325 aa, artificial sequence
  synthetic construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPALFSVGLMDDISPPSTVFAAYNYYAGPKEIRIYPYNNHEGGGSFQAVEQVKFLKKLFEKG

SEQ ID NO: 49 - DNA, 978 bp, Artificial sequence
  synthetic construct
atggcgttcttcgacctgcctctggaagaactgaagaaataccgtccagagcgttacgaagagaaggacttcgacgagttctgggaggaaactctggcggagagcgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggctacatttgcttcgtcatggatactcgtggtcagggttccggctggctgaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctcaggttgaccaggagcgtattgttatcgctggtggctcccagggtggcggcatcgccctggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttttccgcaccctgtcctatttcgacggcgttaacttcgcggctcgtgcaaaaattccggcactgttctctgttggtctgatggacaacatcagccctccttctaccgttttcgcggcatataactattatgcgggtccgaaagaaatccgtatctatccgtacaacaaccacgaaggcggtggtagctttcaggctgttgaacaagtgaaatccctgaagaaactgtttgagaagggctaa

SEQ ID NO: 50 - protein, 325 aa, Artificial sequence
  synthetic construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPALFSVGLMDNISPPSTVFAAYNYYAGPKEIRIYPYNNHEGGGSFQAVEQVKSLKKLFEKG

SEQ ID NO: 51 - DNA, 978 bp, Artificial sequence
  synthetic construct
atggcgttcttcgacctgcctctggaagaactgaagaaataccgtccagagcgttacgaagagaaggacttcgacgagttctgggaggaaactctggcggagaccgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggctacatttgcttcgtcatggatactcgtggtcagggttccggctggctgaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctcaggttgaccaggagcgtattgttatcgctggtggctcccagggtggcggcatcgccctggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttttccgcaccctgtcctatttcgacggcgttaacttcgcggctcgtgcaaaaattccggcactgttctctgttggtctgatggacaacatcagccctccttctaccgttttcgcggcatataactattatgcgggtccgaaagaaatccgtatctatccgtacaacaaccacgaaggcggtggtagctttcaggctgttgaacaagtgaaattcctgaagaaactgtttgagaagggctaa

SEQ ID NO: 52 - protein, 325 aa, Artificial sequence
  synthetic construct
MAFFDLPLEELKKYRPERYEEKDFDEFWEETLAETEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPALFSVGLMDNISPPSTVFAAYNYYAGPKEIRIYPYNNHEGGGSFQAVEQVKFLKKLFEKG

SEQ ID NO: 53 - DNA, 978 bp, Artificial sequence
  synthetic construct
atggcgttcttcgacctgcctctggaagaactgaagaaataccgtccagagcgttacgaagagaaggacttcgacgagttctgggaggaaactctggcggagagcgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggctacatttgcttcgtcatggatactcgtggtcagggttccggctggctgaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctcaggttgacctggagcgtattgttatcgctggtggctcccagggtggcggcatcgccctggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttttccgcaccctgtcctatttcgacggcgttaacttcgcggctcgtgcaaaaattccggcactgttctctgttggtctgatggacaacatcagccctccttctaccgttttcgcggcatataactattatgcgggtccgaaagaaatccgtatctatccgtacaacaaccacgaaggcggtggtagctttcaggctgttgaacaagtgaaattcctgaagaaactgtttgagaagggctaa

SEQ ID NO: 54 - protein, 325 aa, Artificial sequence
  synthetic construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDLERIVIAGGSQGGGIALAVSALSKKAKALLCDVPFLCHFRRAVQLVDTHPYAEITNFLKTHRDKEEIVFRTLSYFDGVNFAARAKIPALFSVGLMDNISPPSTVFAAYNYYAGPKEIRIYPYNNHEGGGSFQAVEQVKFLKKLFEKG

SEQ ID NO: 55 - DNA, 978 bp, artificial sequence
  synthetic construct
atggcgttcttcgacctgcctcgggaagaactgaagaaataccgtccagagcgttacgaagagaaggacttcgacgagttctgggaggaaactctggcggagagcgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggctacatttgcttcgtcatggatactcgtggtcagggttccggctggcagaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctctggttgaccaggagcgtattgatatcgctggtggctcccagggtggcggcatcgccctggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttatccgcaccctgtcctatttcgacggcgttaacttcgcggctcgtgcaaaaattccggcactgttctctgttggtctgatggacaacatcagccctccttctaccgttttcgcggcatataactattatgcgggtctgaaagaaatccgtatctatccgtacaacaaccacgaaggcggtggtagctttcaggctgttgaacaagtgaaattcctgaagaaactgtttgagaagggctaa

SEQ ID NO: 56 - protein, 325 aa, artificial sequence
  Synthetic Construct
MAFFDLPREELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWQKGDTPDYPEGPVDPQYPGFMTRGILDPRTYYYRRVFTDAVRAVEAAASFPLVDQERIDIAGGSQGGGIALAVSALSKKAKALLCDVPFLCHFRRAVQLVDTHPYAEITNFLKTHRDKEEIVIRTLSYFDGVNFAARAKIPALFSVGLMDNISPPSTVFAAYNYYAGLKEIRIYPYNNHEGGGSFQAVEQVKFLKKLFEKG

SEQ ID NO: 57 - DNA, 978 bp, artificial sequence
  synthetic construct
atggcgttcttcgacctgcctctggaagaactgaagaaataccgtccagagcgttacgaagagaaggacttcgacgagttctgggaggaaactctggcggagagcgaaaagtttccgctggacccagtgttcgagcgtatggaatctcacctgaaaaccgtggaggcatatgacgttactttttctggttaccgtggccagcgtatcaaaggctggctgctggttccggaactggaggaagaaaaactgccgtgcgtagttcagtacatcggttacaacggtggccgtggctttccgcacgattggctgttctggccgtctatgggctacatttgcttcgtcatggatactcgtggtcagggttccggctggctgaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctcaggttgaccaggagcgtattgttatcgctggtggctcccagggtggcggcatcgccctggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttttccgcaccctgtcctatttcgacggcgttaacttcgcggctcgtgcaaaaattccggaactgttctctgttggtctgatggacaacatcagccctccttctaccgttttcgcggcatataactattatgcgggtccgaaagaaatccgtatctatccgtacaacaaccacgaaggcggtggtagctttcaggctgttgaacaagtgaaattcctgaagaaactgtttgagaagggctaa

SEQ ID NO: 58 - protein, 325 aa, artificial sequence
  Synthetic Construct
MAFFDLPLEELKKYRPERYEEKDFDEFWEETLAESEKFPLDPVFERMESHLKTVEAYDVTFSGYRGQRIKGWLLVPELEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPELFSVGLMDNISPPSTVFAAYNYYAGPKEIRIYPYNNHEGGGSFQAVEQVKFLKKLFEKG

SEQ ID NO: 59 - DNA, 978 bp, artificial sequence
  synthetic construct
atggcgttcttcgacctgcctctggaagaactgaagaaataccgtccagagcgttacgaagagaaggacttcgacgagtactgggaggaaactctggcggagagcgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggctacatttgcttcgtcatggatactcgtggtcagggttccggctggctgaaaggcgatactccggattatccggagggcccggtagacccgcagtaccctggcttcatgacgcgtggtgttctggatccgcgtacctattactatcgccgcgtttttaccgatgcagttcgtgccgtagaggccgcggcttctttccctcaggttgaccaggagcgtattgttatcgctggtggctcccagggtggcggcatcgccctggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttttccgcaccctgtcctatttcgacggcgttaacttcgcggctcgtgcaaaaattccggtactgttctctgttggtctgatggacaacatcagccctccttctaccgttttcgcggcatataactattatgcgggtccgaaagaaacccgtatctatccgtacaacagccacgaaggcggtggtagctttcaggctgttgaacaagtgaaattcctgaagaaactgtttgagaagggctaa

SEQ ID NO: 60 - protein, 325 aa, artificial sequence
  Synthetic Construct
MAFFDLPLEELKKYRPERYEEKDFDEYWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGVLDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPVLFSVGLMDNISPPSTVFAAYNYYAGPKETRIYPYNSHEGGGSFQAVEQVKFLKKLFEKG

SEQ ID NO: 61 - DNA, 978 bp, artificial sequence
  synthetic construct
atggcgttcttcgacctgcctctggaagaactgaagaaataccgtccagagcgttacgaagagaaggacttcgacgagttctgggaggaaactctggcggagagcgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggctacatttgcttcgtcatggatactcgtggtcagggttccggctggctgaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctcaggttgaccaggagcgtattgttatcgctggtggctcccagggtggcggcatcgcccaggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttttccgcaccctgtcctatttcgacggcgttaacttcgcggctcgtgcaaaaattccggcactgttctctgttggtctgatggacaacatcagccctccttctaccgttttcgcggcatataactattatgcgggtccgaaagaaatccgtatctatccgtacaacaaccacgaaggcggtggtagctttcaggctgttgaacaagtgaaattcctgaagaaactgtttgagaagggctaa

SEQ ID NO: 62 - protein, 325 aa, artificial sequence
  Synthetic Construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIAQAVSALSKKAKALLCDVPFLCHFRRAVQLVDTHPYAEITNFLKTHRDKEEIVFRTLSYFDGVNFAARAKIPALFSVGLMDNISPPSTVFAAYNYYAGPKEIRIYPYNNHEGGGSFQAVEQVKFLKKLFEKG

SEQ ID NO: 63 - DNA, 978 bp, artificial sequence
  synthetic construct
atggcgttcttcgacctgcctctggaagaactgaagaaataccgtccagagcgttacgaagagaaggacttcgacgagttctgggaggaaactctggcggagagcgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggcttcatttgcttcgtcatggatactcgtggtcagggttccggctggctgaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctcaggttgaccaggagcgtattgttatcgctggtggctcccagggtggcggcatcgccctggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttttccgcaccctgtcctatttcgacggcgttaacttcgcggctcgtgcaaaaattccggcactgttctctgttggtctgatggacaacatcagccctccttctaccgttttcgcggcatataactattatgcgggtccgaaagaaatccgtatctatccgtacaacaaccacgaaggcggtggtagctttcaggctgttgaacaagtgaaattcctgaagaaactgtttgagaagggctaa

SEQ ID NO: 64 - protein, 325 aa, artificial sequence
  Synthetic Construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGFICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPALFSVGLMDNISPPSTVFAAYNYYAGPKEIRIYPYNNHEGGGSFQAVEQVKFLKKLFEKG

SEQ ID NO: 65 - protein, 12 aa, artificial sequence
  Synthetic construct
RVPNKTVTVDGA

SEQ ID NO: 66 - protein, 12 aa, artificial sequence
  Synthetic construct
DRHKSKYSSTKS

SEQ ID NO: 67 - protein, 12 aa, artificial sequence
  Synthetic construct
KNFPQQKEFPLS

SEQ ID NO: 68 - protein, 12 aa, artificial sequence
  Synthetic construct
QRNSPPAMSRRD

SEQ ID NO: 69 - protein, 12 aa, artificial sequence
  Synthetic construct
TRKPNMPHGQYL

SEQ ID NO: 70 - protein, 12 aa, artificial sequence
  Synthetic construct
KPPHLAKLPFTT

SEQ ID NO: 71 - protein, 12 aa, artificial sequence
  Synthetic construct
NKRPPTSHRIHA

SEQ ID NO: 72 - protein, 12 aa, artificial sequence
  Synthetic construct
NLPRYQPPCKPL

SEQ ID NO: 73 - protein, 12 aa, artificial sequence
  Synthetic construct
RPPWKKPIPPSE

SEQ ID NO: 74 - protein, 12 aa, artificial sequence
  Synthetic construct
RQRPKDHFFSRP

SEQ ID NO: 75 - protein, 12 aa, artificial sequence
  Synthetic construct
SVPNKXVTVDGX

SEQ ID NO: 76 - protein, 12 aa, artificial sequence
  Synthetic construct
TTKWRHRAPVSP

SEQ ID NO: 77 - protein, 12 aa, artificial sequence
  Synthetic construct
WLGKNRIKPRAS

SEQ ID NO: 78 - protein, 12 aa, artificial sequence
  Synthetic construct
SNFKTPLPLTQS

SEQ ID NO: 79 - protein, 12 aa, artificial sequence
  Synthetic construct
SVSVGMKPSPRP

SEQ ID NO: 80 - protein, 7 aa, artificial sequence
  Synthetic construct
DLHTVYH

SEQ ID NO: 81 - protein, 7 aa, artificial sequence
  Synthetic construct
HIKPPTR

SEQ ID NO: 82 - protein, 7 aa, artificial sequence
  Synthetic construct
HPVWPAI

SEQ ID NO: 83 - protein, 7 aa, artificial sequence
  Synthetic construct
MPLYYLQ

SEQ ID NO: 84 - protein, 26 aa, artificial sequence
  Synthetic construct
HLTVPWRGGGSAVPFYSHSQITLPNH

SEQ ID NO: 85 - protein, 41 aa, artificial sequence
  Synthetic construct
GPHDTSSGGVRPNLHHTSKKEKRENRKVPFYSHSVTSRGNV

SEQ ID NO: 86 - protein, 7 aa, artificial sequence
  Synthetic construct
KHPTYRQ

SEQ ID NO: 87 - protein, 7 aa, artificial sequence
  Synthetic construct
HPMSAPR

SEQ ID NO: 88 - protein, 7 aa, artificial sequence
  Synthetic construct
MPKYYLQ

SEQ ID NO: 89 - protein, 7 aa, artificial sequence
  Synthetic construct
MHAHSIA

SEQ ID NO: 90 - protein, 12 aa, artificial sequence
  Synthetic construct
AKPISQHLQRGS

SEQ ID NO: 91 - protein, 12 aa, artificial sequence
  Synthetic construct
APPTPAAASATT

SEQ ID NO: 92 - protein, 12 aa, artificial sequence
  Synthetic construct
DPTEGARRTIMT

SEQ ID NO: 93 - protein, 12 aa, artificial sequence
  Synthetic construct
LDTSFPPVPFHA

SEQ ID NO: 94 - protein, 12 aa, artificial sequence
  Synthetic construct
LDTSFHQVPFHQ

SEQ ID NO: 95 - protein, 11 aa, artificial sequence
  Synthetic construct
LPRIANTWSPS

SEQ ID NO: 96 - protein, 12 aa, artificial sequence
  Synthetic construct
RTNAADHPAAVT

SEQ ID NO: 97 - protein, 12 aa, artificial sequence
  Synthetic construct
SLNWVTIPGPKI

SEQ ID NO: 98 - protein, 12 aa, artificial sequence
  Synthetic construct
TDMQAPTKSYSN

SEQ ID NO: 99 - protein, 12 aa, artificial sequence
  Synthetic construct
TIMTKSPSLSCG

SEQ ID NO: 100 - protein, 12 aa, artificial sequence
  Synthetic construct
TPALDGLRQPLR

SEQ ID NO: 101 - protein, 12 aa, artificial sequence
  Synthetic construct
TYPASRLPLLAP

SEQ ID NO: 102 - protein, 12 aa, artificial sequence
  Synthetic construct
AKTHKHPAPSYS

SEQ ID NO: 103 - protein, 12 aa, artificial sequence
  Synthetic construct
TDPTPFSISPER

SEQ ID NO: 104 - protein, 12 aa, artificial sequence
  Synthetic construct
SQNWQDSTSYSN

SEQ ID NO: 105 - protein, 12 aa, artificial sequence
  Synthetic construct
WHDKPQNSSKST

SEQ ID NO: 106 - protein, 12 aa, artificial sequence
  Synthetic construct
LDVESYKGTSMP

SEQ ID NO: 107 - protein, 7 aa, artificial sequence
  Synthetic construct
NTPKENW

SEQ ID NO: 108 - protein, 7 aa, artificial sequence
  Synthetic construct
NTPASNR

SEQ ID NO: 109 - protein, 7 aa, artificial sequence
  Synthetic construct
PRGMLST

SEQ ID NO: 110 - protein, 7 aa, artificial sequence
  Synthetic construct
PPTYLST

SEQ ID NO: 111 - protein, 12 aa, artificial sequence
  Synthetic construct
TIPTHRQHDYRS

SEQ ID NO: 112 - protein, 7 aa, artificial sequence
  Synthetic construct
TPPTHRL

SEQ ID NO: 113 - protein, 7 aa, artificial sequence
  Synthetic construct
LPTMSTP

SEQ ID NO: 114 - protein, 7 aa, artificial sequence
  Synthetic construct
LGTNSTP

SEQ ID NO: 115 - protein, 12 aa, artificial sequence
  Synthetic construct
TPLTGSTNLLSS

SEQ ID NO: 116 - protein, 7 aa, artificial sequence
  Synthetic construct
TPLTKET

SEQ ID NO: 117 - protein, 7 aa, artificial sequence
  Synthetic construct
KQSHNPP

SEQ ID NO: 118 - protein, 7 aa, artificial sequence
  Synthetic construct
QQSHNPP

SEQ ID NO: 119 - protein, 7 aa, artificial sequence
  Synthetic construct
TQPHNPP

SEQ ID NO: 120 - protein, 12 aa, artificial sequence
  Synthetic construct
STNLLRTSTVHP

SEQ ID NO: 121 - protein, 12 aa, artificial sequence
  Synthetic construct
HTQPSYSSTNLF

SEQ ID NO: 122 - protein, 7 aa, artificial sequence
  Synthetic construct
SLLSSHA

SEQ ID NO: 123 - protein, 12 aa, artificial sequence
  Synthetic construct
QQSSISLSSHAV

SEQ ID NO: 124 - protein, 7 aa, artificial sequence
  Synthetic construct
NASPSSL

SEQ ID NO: 125 - protein, 7 aa, artificial sequence
  Synthetic construct
HSPSSLR

SEQ ID NO: 126 - protein, 7 aa, artificial sequence
  Synthetic construct
KXSHHTH

SEQ ID NO: 127 - protein, 7 aa, artificial sequence
  Synthetic construct
EXSHHTH

SEQ ID NO: 128 - protein, 12 aa, artificial sequence
  Synthetic construct
SHHTHYGQPGPV

SEQ ID NO: 129 - protein, 7 aa, artificial sequence
  Synthetic construct
LESTSLL

SEQ ID NO: 130 - protein, 7 aa, artificial sequence
  Synthetic construct
DLTLPFH

SEQ ID NO: 131 - protein, 8 aa, artificial sequence
  Synthetic construct
RTNAADHP

SEQ ID NO: 132 - protein, 12 aa, artificial sequence
  Synthetic construct
IPWWNIRAPLNA

SEQ ID NO: 133 - protein, 18 aa, artificial sequence
  Synthetic construct
EQISGSLVAAPWEGEGER

SEQ ID NO: 134 - protein, 12 aa, artificial sequence
  synthetic hair-binding peptide
TPPELLHGAPRS

SEQ ID NO: 135 - protein, 18 aa, artificial sequence
  Synthetic construct
LDTSFHQVPFHQKRKRKD

SEQ ID NO: 136 - protein, 18 aa, artificial sequence
  Synthetic construct
EQISGSLVAAPWKRKRKD

SEQ ID NO: 137 - protein, 18 aa, artificial sequence
  Synthetic construct
TPPELLHGDPRSKRKRKD

SEQ ID NO: 138 - protein, 13 aa, artificial sequence
  Synthetic construct
NTSQLSTEGEGED

SEQ ID NO: 139 - protein, 13 aa, artificial sequence
  Synthetic construct
TPPELLHGDPRSC

SEQ ID NO: 140 - protein, 20 aa, artificial sequence
  synthetic hair-binding peptide
HINKTNPHQGNHHSEKTQRQ

SEQ ID NO: 141 - protein, 15 aa, artificial sequence
  Synthetic construct
HAHKNQKETHQRHAA

SEQ ID NO: 142 - protein, 15 aa, artificial sequence
  Synthetic construct
HEHKNQKETHQRHAA

SEQ ID NO: 143 - protein, 20 aa, artificial sequence
  Synthetic construct
HNHMQERYTEPQHSPSVNGL

SEQ ID NO: 144 - protein, 17 aa, artificial sequence
  Synthetic construct
THSTHNHGSPRHTNADA

SEQ ID NO: 145 - protein, 20 aa, artificial sequence
  synthetic hair-binding peptide
GSCVDTHKADSCVANNGPAT

SEQ ID NO: 146 - protein, 20 aa, artificial sequence
  synthetic hair-binding peptide
AQSQLPDKHSGLHERAPQRY

SEQ ID NO: 147 - protein, 20 aa, artificial sequence
  Synthetic construct
AQSQLPAKHSGLHERAPQRY

SEQ ID NO: 148 - protein, 20 aa, artificial sequence
  Synthetic construct
AQSQLPEKHSGLHERAPQRY

SEQ ID NO: 149 - protein, 20 aa, artificial sequence
  synthetic hair-binding peptide
TDMMHNHSDNSPPHRRSPRN

SEQ ID NO: 150 - protein, 20 aa, artificial sequence
  synthetic hair-binding peptide
TPPELAHTPHHLAQTRLTDR

SEQ ID NO: 151 - protein, 12 aa, artificial sequence
  Synthetic construct
RLLRLLRLLRLL

SEQ ID NO: 152 - protein, 12 aa, artificial sequence
  Synthetic construct
TPPELLHGEPRS

SEQ ID NO: 153 - protein, 12 aa, artificial sequence
  Synthetic construct
TPPELLHGAPRS

SEQ ID NO: 154 - protein, 12 aa, artificial sequence
  Synthetic construct
EQISGSLVAAPW

SEQ ID NO: 155 - protein, 12 aa, artificial sequence
  Synthetic construct
NEVPARNAPWLV

SEQ ID NO: 156 - protein, 13 aa, artificial sequence
  Synthetic construct
NSPGYQADSVAIG

SEQ ID NO: 157 - protein, 12 aa, artificial sequence
  Synthetic construct
AKPISQHLQRGS

SEQ ID NO: 158 - protein, 12 aa, artificial sequence
  Synthetic construct
LDTSFPPVPFHA

SEQ ID NO: 159 - protein, 12 aa, artificial sequence
  Synthetic construct
SLNWVTIPGPKI

SEQ ID NO: 160 - protein, 12 aa, artificial sequence
  Synthetic construct
TQDSAQKSPSPL

SEQ ID NO: 161 - protein, 12 aa, artificial sequence
  Synthetic construct
KELQTRNVVQRE

SEQ ID NO: 162 - protein, 12 aa, artificial sequence
  Synthetic construct
QRNSPPAMSRRD

SEQ ID NO: 163 - protein, 12 aa, artificial sequence
  Synthetic construct
TPTANQFTQSVP

SEQ ID NO: 164 - protein, 12 aa, artificial sequence
  Synthetic construct
AAGLSQKHERNR

SEQ ID NO: 165 - protein, 12 aa, artificial sequence
  Synthetic construct
ETVHQTPLSDRP

SEQ ID NO: 166 - protein, 12 aa, artificial sequence
  Synthetic construct
KNFPQQKEFPLS

SEQ ID NO: 167 - protein, 12 aa, artificial sequence
  Synthetic construct
LPALHIQRHPRM

SEQ ID NO: 168 - protein, 12 aa, artificial sequence
  Synthetic construct
QPSHSQSHNLRS

SEQ ID NO: 169 - protein, 12 aa, artificial sequence
  Synthetic construct
RGSQKSKPPRPP

SEQ ID NO: 170 - protein, 12 aa, artificial sequence
  Synthetic construct
THTQKTPLLYYH

SEQ ID NO: 171 - protein, 12 aa, artificial sequence
  Synthetic construct
TKGSSQAILKST

SEQ ID NO: 172 - protein, 7 aa, artificial sequence
  Synthetic construct
TAATTSP

SEQ ID NO: 173 - protein, 7 aa, artificial sequence
  Synthetic construct
LGIPQNL

SEQ ID NO: 174 - protein, 20 aa, artificial sequence
  Synthetic construct
THSTHNHGSPRHTNADAGNP

SEQ ID NO: 175 - protein, 20 aa, artificial sequence
  Synthetic construct
QQHKVHHQNPDRSTQDAHHS

SEQ ID NO: 176 - protein, 15 aa, artificial sequence
  Synthetic construct
HHGTHHNATKQKNHV

SEQ ID NO: 177 - protein, 15 aa, artificial sequence
  Synthetic construct
STLHKYKSQDPTPHH

SEQ ID NO: 178 - protein, 12 aa, artificial sequence
  Synthetic construct
SVSVGMKPSPRP

SEQ ID NO: 179 - protein, 12 aa, artificial sequence
  synthetic hair-binding peptide
TPPTNVLMLATK

SEQ ID NO: 180 - protein, 12 aa, artificial sequence
  Synthetic construct
TPPELLHGDPRS

SEQ ID NO: 181 - protein, 7 aa, artificial sequence
  synthetic hair-binding peptide
NTSQLST

SEQ ID NO: 182 - protein, 15 aa, artificial sequence
  Synthetic construct
STLHKYKSQDPTPHH

SEQ ID NO: 183 - protein, 12 aa, artificial sequence
  synthetic hair-binding peptide
GMPAMHWIHPFA

SEQ ID NO: 184 - protein, 15 aa, artificial sequence
  synthetic hair-binding peptide
HDHKNQKETHQRHAA

SEQ ID NO: 185 - protein, 20 aa, artificial sequence
  Synthetic construct
HNHMQERYTDPQHSPSVNGL

SEQ ID NO: 186 - protein, 20 aa, artificial sequence
  synthetic hair-binding peptide
TAEIQSSKNPNPHPQRSWTN

SEQ ID NO: 187 - protein, 21 aa, artificial sequence
  Dyed-hair-bindg peptide
SSADFASFGFFGFSAASADSR

SEQ ID NO: 188 - protein, 23 aa, artificial sequence
  Dyed-hair-bindg peptide
SSFAEAWSRAWPRAEVFFPSRGY

SEQ ID NO: 189 - protein, 17 aa, artificial sequence
  Dyed-hair-bindg peptide
SSFSVNEPHAWMAPLSR

SEQ ID NO: 190 - protein, 17 aa, artificial sequence
  Dyed-hair-bindg peptide
SSFSWVYGHGGLGFASR

SEQ ID NO: 191 - protein, 17 aa, artificial sequence
  Dyed-hair-bindg peptide
SSFVSWSPYKSPPELSR

SEQ ID NO: 192 - protein, 21 aa, artificial sequence
  Dyed-hair-bindg peptide
SSFYGSSAFVSSGVSVAYGSR

SEQ ID NO: 193 - protein, 21 aa, artificial sequence
  Dyed-hair-bindg peptide
SSGSVAVSAEASWFSGVAASR

SEQ ID NO: 194 - protein, 15 aa, artificial sequence
  Dyed-hair-bindg peptide
SSHDEHYQYHYYSSR

SEQ ID NO: 195 - protein, 15 aa, artificial sequence
  Dyed-hair-bindg peptide
SSHYYYNDYDHQSSR

SEQ ID NO: 196 - protein, 17 aa, artificial sequence
  Dyed-hair-bindg peptide
SSLFNMYGHQSVLGPSR

SEQ ID NO: 197 - protein, 17 aa, artificial sequence
  Dyed-hair-bindg peptide
SSLFSDVHYGSNKALSR

SEQ ID NO: 198 - protein, 17 aa, artificial sequence
  Dyed-hair-bindg peptide
SSLLSDFHYGDMWDASR

SEQ ID NO: 199 - protein, 15 aa, artificial sequence
  Dyed-hair-bindg peptide
SSNYNYNYNYQYSSR

SEQ ID NO: 200 - protein, 21 aa, artificial sequence
  synethetic construct
SSNYNYNYNYQYSSREGEGER

SEQ ID NO: 201 - protein, 21 aa, ARTIFICIAL SEQUENCE
  Synthetic construct
SSNYNYNYNYQYSSRKRKRKD

SEQ ID NO: 202 - protein, 15 aa, artificial sequence
  Dyed-hair-bindg peptide
SSQYYQDYQYYHSSR

SEQ ID NO: 203 - protein, 23 aa, artificial sequence
  Dyed-hair-bindg peptide
SSSCMGSHNPRMSVEESTRNCSR

SEQ ID NO: 204 - protein, 23 aa, artificial sequence
  Dyed-hair-bindg peptide
SSSCNNNWFYSSTLPGGDHACSR

SEQ ID NO: 205 - protein, 23 aa, artificial sequence
  Dyed-hair-bindg peptide
SSSCYDVECSSFVAWMRGPSSSR

SEQ ID NO: 206 - protein, 21 aa, artificial sequence
  Dyed-hair-bindg peptide
SSSFAASSAFSFLVDAVAWSR

SEQ ID NO: 207 - protein, 17 aa, artificial sequence
  Dyed-hair-bindg peptide
SSSFAYLVPDDGWLSSR

SEQ ID NO: 208 - protein, 21 aa, artificial sequence
  Dyed-hair-bindg peptide
SSSGAVFSSGGADAGWGVWSR

SEQ ID NO: 209 - protein, 23 aa, artificial sequence
  Dyed-hair-bindg peptide
SSSSADAAYGHCCGAGFSTFSSR

SEQ ID NO: 210 - protein, 23 aa, artificial sequence
  Dyed-hair-bindg peptide
SSSSDVHNSIIGWDFYHSRGSSR

SEQ ID NO: 211 - protein, 21 aa, artificial sequence
  Dyed-hair-bindg peptide
SSSSLDFFSYSAFSGGVAESR

SEQ ID NO: 212 - protein, 23 aa, artificial sequence
  Dyed-hair-bindg peptide
SSSSNDSNVSWFHYYASGLTSSR

SEQ ID NO: 213 - protein, 21 aa, artificial sequence
  Dyed-hair-bindg peptide
SSVDYEVPLAVAAEWGFSVSR

SEQ ID NO: 214 - protein, 15 aa, artificial sequence
  Dyed-hair-bindg peptide
SSYHYDYDHYYESSR

SEQ ID NO: 215 - protein, 15 aa, artificial sequence
  Dyed-hair-bindg peptide
SSYYNYHYQYQDSSR

SEQ ID NO: 216 - protein, 15 aa, artificial sequence
  Dyed-hair-bindg peptide
SSYYYDYYQQDYSSR

SEQ ID NO: 217 - protein, 12 aa, artificial sequence
  Synthetic construct
KRGRHKRPKRHK

SEQ ID NO: 218 - protein, 7 aa, artificial sequence
  Synthetic construct
RLLRLLR

SEQ ID NO: 219 - protein, 12 aa, artificial sequence
  Synthetic construct
HKPRGGRKKALH

SEQ ID NO: 220 - protein, 18 aa, artificial sequence
  Synthetic construct
KPRPPHGKKHRPKHRPKK

SEQ ID NO: 221 - protein, 18 aa, artificial sequence
  Synthetic construct
RGRPKKGHGKRPGHRARK

SEQ ID NO: 222 - protein, 12 aa, artificial sequence
  Synthetic construct
TPFHSPENAPGS

SEQ ID NO: 223 - protein, 13 aa, artificial sequence
  Synthetic construct
TPFHSPENAPGSK

SEQ ID NO: 224 - protein, 16 aa, artificial sequence
  Synthetic construct
TPFHSPENAPGSGGGS

SEQ ID NO: 225 - protein, 17 aa, artificial sequence
  Synthetic construct
TPFHSPENAPGSGGGSS

SEQ ID NO: 226 - protein, 15 aa, artificial sequence
  Synthetic construct
TPFHSPENAPGSGGG

SEQ ID NO: 227 - protein, 7 aa, artificial sequence
  Synthetic construct
FTQSLPR

SEQ ID NO: 228 - protein, 12 aa, artificial sequence
  Synthetic construct
KQATFPPNPTAY

SEQ ID NO: 229 - protein, 12 aa, artificial sequence
  Synthetic construct
HGHMVSTSQLSI

SEQ ID NO: 230 - protein, 7 aa, artificial sequence
  Synthetic construct
LSPSRMK

SEQ ID NO: 231 - protein, 7 aa, artificial sequence
  Synthetic construct
LPIPRMK

SEQ ID NO: 232 - protein, 7 aa, artificial sequence
  Synthetic construct
HQRPYLT

SEQ ID NO: 233 - protein, 7 aa, artificial sequence
  Synthetic construct
FPPLLRL

SEQ ID NO: 234 - protein, 7 aa, artificial sequence
  Synthetic construct
QATFMYN

SEQ ID NO: 235 - protein, 11 aa, artificial sequence
  Synthetic construct
VLTSQLPNHSM

SEQ ID NO: 236 - protein, 7 aa, artificial sequence
  Synthetic construct
HSTAYLT

SEQ ID NO: 237 - protein, 12 aa, artificial sequence
  Synthetic construct
APQQRPMKTFNT

SEQ ID NO: 238 - protein, 12 aa, artificial sequence
  Synthetic construct
APQQRPMKTVQY

SEQ ID NO: 239 - protein, 7 aa, artificial sequence
  Synthetic construct
PPWLDLL

SEQ ID NO: 240 - protein, 7 aa, artificial sequence
  Synthetic construct
PPWTFPL

SEQ ID NO: 241 - protein, 7 aa, artificial sequence
  Synthetic construct
SVTHLTS

SEQ ID NO: 242 - protein, 7 aa, artificial sequence
  Synthetic construct
VITRLTS

SEQ ID NO: 243 - protein, 12 aa, artificial sequence
  Synthetic construct
DLKPPLLALSKV

SEQ ID NO: 244 - protein, 12 aa, artificial sequence
  Synthetic construct
SHPSGALQEGTF

SEQ ID NO: 245 - protein, 12 aa, artificial sequence
  Synthetic construct
FPLTSKPSGACT

SEQ ID NO: 246 - protein, 12 aa, artificial sequence
  Synthetic construct
DLKPPLLALSKV

SEQ ID NO: 247 - protein, 7 aa, artificial sequence
  Synthetic construct
PLLALHS

SEQ ID NO: 248 - protein, 7 aa, artificial sequence
  Synthetic construct
VPISTQI

SEQ ID NO: 249 - protein, 12 aa, artificial sequence
  Synthetic construct
YAKQHYPISTFK

SEQ ID NO: 250 - protein, 7 aa, artificial sequence
  Synthetic construct
HSTAYLT

SEQ ID NO: 251 - protein, 12 aa, artificial sequence
  Synthetic construct
STAYLVAMSAAP

SEQ ID NO: 252 - protein, 12 aa, artificial sequence
  Synthetic construct
SVSVGMKPSPRP

SEQ ID NO: 253 - protein, 12 aa, artificial sequence
  Synthetic construct
TMGFTAPRFPHY

SEQ ID NO: 254 - protein, 12 aa, artificial sequence
  Synthetic construct
NLQHSVGTSPVW

SEQ ID NO: 255 - protein, 15 aa, artificial sequence
  Synthetic construct
QLSYHAYPQANHHAP

SEQ ID NO: 256 - protein, 12 aa, artificial sequence
  Synthetic construct
NQAASITKRVPY

SEQ ID NO: 257 - protein, 14 aa, artificial sequence
  Synthetic construct
SGCHLVYDNGFCDH

SEQ ID NO: 258 - protein, 14 aa, artificial sequence
  Synthetic construct
ASCPSASHADPCAH

SEQ ID NO: 259 - protein, 14 aa, artificial sequence
  Synthetic construct
NLCDSARDSPRCKV

SEQ ID NO: 260 - protein, 12 aa, artificial sequence
  Synthetic construct
NHSNWKTAADFL

SEQ ID NO: 261 - protein, 12 aa, artificial sequence
  Synthetic construct
GSSTVGRPLSYE

SEQ ID NO: 262 - protein, 12 aa, artificial sequence
  Synthetic construct
SDTISRLHVSMT

SEQ ID NO: 263 - protein, 12 aa, artificial sequence
  Synthetic construct
SPLTVPYERKLL

SEQ ID NO: 264 - protein, 12 aa, artificial sequence
  Synthetic construct
SPYPSWSTPAGR

SEQ ID NO: 265 - protein, 12 aa, artificial sequence
  Synthetic construct
VQPITNTRYEGG

SEQ ID NO: 266 - protein, 12 aa, artificial sequence
  Synthetic construct
WPMHPEKGSRWS

SEQ ID NO: 267 - protein, 14 aa, artificial sequence
  Synthetic construct
DACSGNGHPNNCDR

SEQ ID NO: 268 - protein, 14 aa, artificial sequence
  Synthetic construct
DHCLGRQLQPVCYP

SEQ ID NO: 269 - protein, 14 aa, artificial sequence
  Synthetic construct
DWCDTIIPGRTCHG

SEQ ID NO: 270 - protein, 12 aa, artificial sequence
  Synthetic construct
ALPRIANTWSPS

SEQ ID NO: 271 - protein, 12 aa, artificial sequence
  Synthetic construct
YPSFSPTYRPAF

SEQ ID NO: 272 - protein, 8 aa, Artificial Sequence
  Synthetic construct - caspace 3 cleavable linker
LESGDEVD

SEQ ID NO: 273 - protein, 37 aa, Artificial Sequence
  synthetic construct
TSTSKASTTTTSSKTTTTSSKTTTTTSKTSTTSSSST

SEQ ID NO: 274 - protein, 22 aa, Artificial Sequence
  synthetic construct
GQGGYGGLGSQGAGRGGLGGQG

SEQ ID NO: 275 - protein, 10 aa, Artificial Sequence
  synthetic construct
GPGGYGPGQQ

SEQ ID NO: 276 - protein, 9 aa, Artificial Sequence
  synthetic construct
GGSGPGSGG

SEQ ID NO: 277 - protein, 5 aa, Artificial Sequence
  synthetic construct
GGPKK

SEQ ID NO: 278 - protein, 5 aa, Artificial Sequence
  synthetic construct
GPGVG

SEQ ID NO: 279 - protein, 7 aa, Artificial Sequence
  synthetic construct
GGGCGGG

SEQ ID NO: 280 - protein, 4 aa, Artificial Sequence
  synthetic construct
GGGC

SEQ ID NO: 281 - protein, 14 aa, Artificial Sequence
  synthetic construct
PHMASMTGGQQMGS

SEQ ID NO: 282 - protein, 16 aa, Artificial Sequence
  synthetic construct
GPEEAAKKEEAAKKPA

SEQ ID NO: 283 - protein, 14 aa, Artificial Sequence
  synthetic construct
GSGGGGSGSGGGGS

SEQ ID NO: 284 - protein, 37 aa, Artificial Sequence
  synthetic construct
GPEPEPEPEPIPEPPKEAPVVIEKPKPKPKPKPKPPA

SEQ ID NO: 285 - protein, 18 aa, artificial sequence
  synthetic construct
GPGSGGAGSPGSAGGPGS

SEQ ID NO: 286 - DNA, 1293 bp, artificial sequence
  synthetic construct
atggcattcttcgacctgccactggaggagctgaagaaatatcgtcctgagcgctatgaagaaaaggattttgatgagttctgggaggaaactctggcagaaagcgagaaattcccgctggacccggtgtttgaacgtatggagagccatttgaaaaccgtggaagcctacgatgtgacctttagcggctatcgtggtcaacgcattaaaggttggctgctggtgcctaagctggaagaagagaagttgccttgcgtggtgcaatacattggttacaacggtggtcgtggttttccgcacgattggttgttctggccgagcatgggttacatttgctttgtgatggatacccgcggtcaaggtagcggttggctgaaaggcgacaccccggattacccggagggtccagtcgacccacagtacccgggttttatgacccgtggtatccttgacccgcgtacctactactaccgtcgtgtgttcaccgacgcggtacgtgcagttgaggcagccgcgtccttcccacaggttgaccaggaacgcatcgtgattgcgggtggctcgcaaggtggtggtatcgcattggcggttagcgctctgtccaagaaagcaaaagcactgctgtgcgacgtgccgtttctgtgtcacttccgtcgtgcagttcagctggttgatacgcacccttacgccgaaattaccaactttctgaaaacgcaccgcgataaggaagaaatcgtgttccgcaccctgagctattttgacggcgtcaatttcgcagcgcgtgcgaagattccagcgttgttcagcgttggtctgatggataacatttccccgccttctaccgttttcgcggcctacaactactacgcaggcccgaaagagattcgcatctacccatataacaaccatgagggtggcggtagcttccaggcagttgagcaagttaagttcctgaagaagctgttcgaaaagggtggtccgggttcgggtggtgcgggcagcccgggtagcgccggtggccctggatcccctagcgcacaaagccaactgccggacaagcatagcggcctgcacgaacgtgctccgcagcgttacggtccggaaccggaaccggaaccggagccgatcccagaaccgccgaaagaggccccagttgttattgaaaagccgaagccgaaaccgaagccgaagccgaagccgcctgcgcatgatcataagaatcagaaggaaacccatcagcgtcacgccgctggttcgggcggtggtggtagcccgcaccatcaccaccaccac

SEQ ID NO: 287 - DNA, 1305 bp, artificial sequence
  synthetic construct
atggcattcttcgacctgccactggaggagctgaagaaatatcgtcctgagcgctatgaagaaaaggattttgatgagttctgggaggaaactctggcagaaagcgagaaattcccgctggacccggtgtttgaacgtatggagagccatttgaaaaccgtggaagcctacgatgtgacctttagcggctatcgtggtcaacgcattaaaggttggctgctggtgcctaagctggaagaagagaagttgccttgcgtggtgcaatacattggttacaacggtggtcgtggttttccgcacgattggttgttctggccgagcatgggttacatttgctttgtgatggatacccgcggtcaaggtagcggttggctgaaaggcgacaccccggattacccggagggtccagtcgacccacagtacccgggttttatgacccgtggtatccttgacccgcgtacctactactaccgtcgtgtgttcaccgacgcggtacgtgcagttgaggcagccgcgtccttcccacaggttgaccaggaacgcatcgtgattgcgggtggctcgcaaggtggtggtatcgcattggcggttagcgctctgtccaagaaagcaaaagcactgctgtgcgacgtgccgtttctgtgtcacttccgtcgtgcagttcagctggttgatacgcacccttacgccgaaattaccaactttctgaaaacgcaccgcgataaggaagaaatcgtgttccgcaccctgagctattttgacggcgtcaatttcgcagcgcgtgcgaagattccagcgttgttcagcgttggtctgatggataacatttccccgccttctaccgttttcgcggcctacaactactacgcaggcccgaaagagattcgcatctacccatataacaaccatgagggtggcggtagcttccaggcagttgagcaagttaagttcctgaagaagctgttcgaaaagggtggtccgggttcgggtggtgcgggcagcccgggtagcgccggtggccctggatccgcgcaaagccaactgccggacaaacatagcggtctgcacgagcgtgcgccgcagcgttacggtagcggtacggcggaaattcaatccagcaagaacccgaacccgcacccgcagcgcagctggaccaatggcagcggtcataatcacatgcaagagcgttacacggacccgcagcacagcccgagcgttaatggtttgggtagcggccacgaccataagaatcagaaagaaacccatcaacgccacgcggcgtccagccaccaccaccatcaccac

SEQ ID NO: 288 - protein, 431 aa, artificial sequence
  synthetic construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPALFSVGLMDNISPPSTVFAAYNYYAGPKEIRIYPYNNHEGGGSFQAVEQVKFLKKLFEKGGPGSGGAGSPGSAGGPGSPSAQSQLPDKHSGLHERAPQRYGPEPEPEPEPIPEPPKEAPVVIEKPKPKPKPKPKPPAHDHKNQKETHQRHAAGSGGGGSPHHHHHH

SEQ ID NO: 289 - protein, 435 aa, artificial sequence
  synthetic construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPALFSVGLMDNISPPSTVFAAYNYYAGPKEIRIYPYNNHEGGGSFQAVEQVKFLKKLFEKGGPGSGGAGSPGSAGGPGSAQSQLPDKHSGLHERAPQRYGSGTAEIQSSKNPNPHPQRSWTNGSGHNHMQERYTDPQHSPSVNGLGSGHDHKNQKETHQRHAASSHHHHHH

SEQ ID NO: 290 - protein, 88 aa, artificial sequence
  synthetic construct
PSAQSQLPDKHSGLHERAPQRYGPEPEPEPEPIPEPPKEAPVVIEKPKPKPKPKPKPPAHDHKNQKETHQRHAAGSGGGGSPHHHHHH

SEQ ID NO: 291 - protein, 92 aa, artificial sequence
  synthetic construct
AQSQLPDKHSGLHERAPQRYGSGTAEIQSSKNPNPHPQRSWTNGSGHNHMQERYTDPQHSPSVNGLGSGHDHKNQKETHQRHAASSHHHHHH

SEQ ID NO: 292 - DNA, 6368 bp, artificial sequence
  synthetic construct
agatctcgatcccgcgaaattaatacgactcactatagggagaccacaacggtttccctctagaaataattttgtttaactttaagaaggagatatacatatgcacactccagaacatatcaccgcagtagtacagcgttttgtggcagctctgaacgcgggcgagctggaaggtattgtggcgctgttcgcggaagaagccaccgtggaagaaccggtgggttctgaaccgcgttccggcaccgcagcctgccgtgaattttacgcaaacagcctgaagctgccgctggcggttgaactgacccaagaatgtcgtgcggtggctaacgaagccgctttcgcgttcaccgtgtccttcgaataccagggtcgtaagaccgttgtggcgccatgcgaacactttcgtttcaacggcgcaggcaaagtggtttccatccgcgcactgttcggtgaaaagaacatccatgcttgtcagggatccgatccgactccgccgacgaatgtactgatgctggcaaccaaaggcggtggtacgcattccacgcacaaccatggcagcccgcgccacacgaatgctgacgcaggcaatccgggcggcggcaccccaccaaccaatgtcctgatgctggctactaaaggcggcggcacgcattctacccacaaccatggtagcccgcgccatactaatgcagatgccggcaacccgggcggtggtaccccgccaaccaacgttctgatgctggcgacgaaaggtggcggtacccattccacgcataatcatggcagccctcgccacaccaacgctgatgctggtaatcctggtggcggtaagaagaaataataaggcgcgccgacccagctttcttgtacaaagtggttgattcgaggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgatcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcg

SEQ ID NO: 293 - protein, 325 aa, Thermotoga maritima
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPALFSVGLMDNISPPSTVFAAYNYYAGPKEIRIYPYNNHEGGGSFQAVEQVKFLKKLFEKG

SEQ ID NO: 294 - protein, 431 aa, artificial sequence
  synthetic construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGGTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPALFSVGLMDNISPPSTVFAAYNYYAGPKEIRIYPYNNHEGGGSFQAVEQVKFLKKLFEKGGPGSGGAGSPGSAGGPGSPSAQSQLPDKHSGLHERAPQRYGPEPEPEPEPIPEPPKEAPVVIEKPKPKPKPKPKPPAHDHKNQKETHQRHAAGSGGGGSPHHHHHH

SEQ ID NO: 295 - protein, 435 aa, artificial sequence
  synthetic construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGGTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPALFSVGLMDNISPPSTVFAAYNYYAGPKEIRIYPYNNHEGGGSFQAVEQVKFLKKLFEKGGPGSGGAGSPGSAGGPGSAQSQLPDKHSGLHERAPQRYGSGTAEIQSSKNPNPHPQRSWTNGSGHNHMQERYTDPQHSPSVNGLGSGHDHKNQKETHQRHAASSHHHHHH

SEQ ID NO: 296 - DNA, 978 bp, artificial sequence
  synthetic construct
atggcgttcttcgacctgcctctggaagaactgaagaaataccgtccagagcgttacgaagagaaggacttcgacgagttctgggaggaaactctggcggagagcgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggctacatttgcttcgtcatggatactcgtggtcagggttccggctggctgaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctcaggttgaccaggagcgtattgttatcgctggtggctcccagggtggcggcatcgccctggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttttccgcaccctgtcctatttcgacggcgttaacttcgcggctcgtgcaaaaattccggcactgtcctctgttggtctgatggacaacatcacccctccttctaccgttttcgcggcatataactattatgcgggtccgaaagaaatccgtatctatccgtacaacaaccacgaaggcggtggtagctttcaggctgttgaacaagtgaaattcctgaagaaactgtttgagaagggctaa

SEQ ID NO: 297 - protein, 325 aa, artificial sequence
  Synthetic Construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPALSSVGLMDNITPPSTVFAAYNYYAGPKEIRIYPYNNHEGGGSFQAVEQVKFLKKLFEKG

SEQ ID NO: 298 - DNA, 978 bp, artificial sequence
  synthetic construct
atggcgttcttcgacctgcctctggaagaactgaagaaataccgtccagagcgttacgaagagaaggacttcgacgagttctgggaggaaactctggcggagagcgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggctacatttgcttcgtcatggatactcgtggtcagggttccggctggctgaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctcaggttgaccaggagcgtattgttatcgctggtggctcccagggtggcggcatcgccctggcggtatctgcgctgagcaagaaagctaaggcactgctgtgtgacgtcccgttcctgtgtcacttctgtcgcgctgttcagctggtagatacccatccgtacgcggagattactaacttcctgaaaactcaccgcgacaaagaagaaatcgttttccgcaccctgtcctatttcgacggcgttaacttcgcggctcgtgcaaaaattccggcactgttctctgttggtctgatggacaacatcacccctccttctaccgttttcgcggcatataactattatgcgggtccgaaagaaatccgtatctatccgtacaacaaccacgaaggcggtggtagctttcaggctgttgaacaagtgaaactcctgaagaaactgtttgagaagggctaa

SEQ ID NO: 299 - protein, 325 aa, artificial sequence
  Synthetic Construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFCRAVQLVDTHPYAEITNFLKTHRDKEEIVFRTLSYFDGVNFAARAKIPALFSVGLMDNITPPSTVFAAYNYYAGPKEIRIYPYNNHEGGGSFQAVEQVKLLKKLFEKG

SEQ ID NO: 300 - DNA, 978 bp, artificial sequence
  synthetic construct
atggcgttcttcgacctgcctctggaagaactgaagaaataccgtccagagcgttacgaagagaaggacttcgacgagttctgggaggaaactctggcggagagcgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggctacatttgcttcgtcatggatactcgtggtcagggttccggctggctgaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctcaggttgaccaggagcgtattgttatcgctggtggctcccagggtggcggcatcgccctggcggtatctgcgctgagcaagaaagctaaggcactgctgtgtgacgtcccgttcctgtgtcacttccgtcgcgctgttcagctggtagataccctaccgtacgcggagattgctaacttcctgaaaactcaccgcgacaaagaagaaatcgttttccgcaccctgtcctatttcgacggcgttaacttcgcggctcgtgcaaaaattccggcactgttctctgttggtctgatggacaacatcacccctccttctaccgttttcgcggcatataactattatgtgggtccgaaagaaatccgtatctatccgtacaacaaccacgaaggcggtggtagctttcaggctgttgaacaagtgaaattcctgaagaaactgtttgagaagggctaa

SEQ ID NO: 301 - protein, 325 aa, artificial sequence
  Synthetic Construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTLPYAEIANFLKTHRDKEEIVFRTLSYFDGVNFAARAKIPALFSVGLMDNITPPSTVFAAYNYYVGPKEIRIYPYNNHEGGGSFQAVEQVKFLKKLFEKG

SEQ ID NO: 302 - DNA, 978 bp, artificial sequence
  synthetic construct
atggcgttcttcgacctgcctctggaagaactgaagaaataccgtccagagcgttacgaagagaaggacttcgacgagttctgggaggaaactctggcggagagcgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggctacatttgcttcgtcatggatactcgtggtcagggttccggctggctgaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctcaggttgaccaggagcgtattgttatcgctggtggctcccagggtggcggcatcgccctggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttttccgcaccctgtcctatttcggcggcgttaacttcgcggctcgtgcaaaaattccggcactgttctctgttggtctgatggacaacatcacccctccttctaccgttttcgcggcatataactattatgcgggtccgaaagaaatccgtatctatccgtacaacaaccacgaaggcggtggtagctttcaggctgttgaacaagtgaaattcctgaagaaactgtttgagaagggctaa

SEQ ID NO: 303 - protein, 325 aa, artificial sequence
  Synthetic Construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFGGVNFAARAKIPALFSVGLMDNITPPSTVFAAYNYYAGPKEIRIYPYNNHEGGGSFQAVEQVKFLKKLFEKG

SEQ ID NO: 304 - DNA, 978 bp, artificial sequence
  synthetic construct
atggcgttcttcgacctgcctctggaagaactgaagaaataccgtccagagcgttacgaagagaaggacttcgacgagttctgggaggaaactctggcggagagcgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggctacatttgcttcgtcatggatactcgtggtcagggttccggctggctgaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctcaggttgaccaggagcgtattgttatcgctggtggctcccagggtggcggcatcgccctggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttttccgcaccctgtcctatttcgacggcgttaacttcgcggctagtgcaaaatttccggcactgttctctgttggtctgatggacaacatcacccctccttctaccgttttcgcggcatataactattatgcgggtccgaaagaaatccgtatctatccgtacaacaaccacgaaggcggtggtagctttcaggctgttgaacaagtgaaattcctgaagaaactgtttgagaagggctaa

SEQ ID NO: 305 - protein, 325 aa, artificial sequence
  Synthetic Construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAASAKFPALFSVGLMDNITPPSTVFAAYNYYAGPKEIRIYPYNNHEGGGSFQAVEQVKFLKKLFEKG

SEQ ID NO: 306 - DNA, 978 bp, artificial sequence
  synthetic construct
atggcgttcttcgacctgcctctggaagaactgaagaaataccgtccagagcgttacgaagagaaggacttcgacgagttctgtgaggaaactctggcggagagcgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgtcctggccgtctatgggctacatttgcttcgtcatggatactcgtggtcagggttccggctggctgaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctcaggttgaccaggagcgtattgttatcgctggtggctcccagggtggcggcatcgccctggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttttccgcaccctgtcctatttcgacggcgttaacttcgcggctcgtgcaaaaattccggcactgttctctgttggtctgatggacaacatcacccctccttctaccgttttcgcggcatataactattatgcgggtccgaaagaaatccgtatctatccgtacaacaaccacgaaggcggtggtagctttcaggctgttgaacaagtgaaattcctgaagaaactgtttgagaagggctaa

SEQ ID NO: 307 - protein, 325 aa, artificial sequence
  Synthetic Construct
MAFFDLPLEELKKYRPERYEEKDFDEFCEETLAESEKFPLDPVFERMESHLKTVEAYDVTFSGYRGQRIKGWLLVPKLEEEKLPCVVQYIGYNGGRGFPHDWLSWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPALFSVGLMDNITPPSTVFAAYNYYAGPKEIRIYPYNNHEGGGSFQAVEQVKFLKKLFEKG

SEQ ID NO: 308 - DNA, 978 bp, artificial sequence
  synthetic construct
atggcgttcttcgacctgcctctggaagaactgaagaaataccgtccagagcgttacgaagagaaggacttcgacgagttctgggaggaaactctggcggagagcgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggctacatttgcttcgtcatggatactcgtggtcagggttccggctggctgaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctcaggttgaccaggagcgtattgttatcgctggtggctcccagggtggcggcatcgccctggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttttccgcaccctgtcctatttcgacggcgttaacttcgcggctcgtgcaaaaattccgccactgttctctgttggtctgatggacaacatcagccctccttctaccgttttcgcggcatataactattatgcgggtccgaaagaaatccgtatctatccgtacaacaaccacgaaggcggtggtagctttcaggctgttgaacaagtgaaattcctgaagaaactgtttgagaagggctaa

SEQ ID NO: 309 - protein, 325 aa, artificial sequence
  Synthetic Construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPPLFSVGLMDNISPPSTVFAAYNYYAGPKEIRIYPYNNHEGGGSFQAVEQVKFLKKLFEKG

SEQ ID NO: 310 - DNA, 978 bp, artificial sequence
  synthetic construct
atggcgttcttcgacctgcctctggaagaactgaagaaataccgtccagagcgttacgaagagaaggacttcgacgagttctgtgaggaaactccggcggagagcgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggctacatttgcttcgtcatggatactcgtggtcagggttccggctggctgaaaggcgatactccggattatccggagggcccggtagacccgcagtaccctggcttcatgacgcgtggtattctggaaccgcgtacctattactatcgccgcgtttttaccgatgcagttcgtgccgtagaggccgcggcttctttccctcaggttgaccaggagcgtattgttatcgctggtggctcccagggtggcggcatcgccctggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttttccgcaccctgtcctatttcgacggcgttaacttcgcggctcgtgcaaaaattccggcactgttctctgttggtctgatggacaacatcacccctccttctaccgttttcgcggcatataactattatgcgggtccgaaagaaatccgtatctatccgtacaacaaccacgaaggcggtggtagctttcaggctgttgaacaagtgaaattcctgaagaaactgtttgagaagggctaa

SEQ ID NO: 311 - protein, 325 aa, artificial sequence
  Synthetic Construct
MAFFDLPLEELKKYRPERYEEKDFDEFCEETPAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILEPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPALFSVGLMDNITPPSTVFAAYNYYAGPKEIRIYPYNNHEGGGSFQAVEQVKFLKKLFEKG

SEQ ID NO: 312 - protein, 88 aa, artificial sequence
  synthetic construct
PSAQSQLPDRHSGLHERAPQRYGPEPEPEPEPIPEPPREAPVVIERPRPRPRPRPRPPAHDHRNQRETHQRHAAGSGGGGSPHHHHHH

SEQ ID NO: 313 - protein, 16 aa, artificial sequence
  synthetic construct
GKGKGKGKGKHHHHHH

SEQ ID NO: 314 - protein, 216 aa, Mycobacterium smegmatis
MAKRILCFGDSLTWGWVPVEDGAPTERFAPDVRWTGVLAQQLGADFEVIEEGLVARTTNIDDPTDPRLNGASYLPSCLATHLPLDLVIIMLGTNDTKAYFRRTPLDIALGMSVLVTQVLTSAGGVGTTYPAPKVLVVSPPPLAPMPHPWFQLIFEGGEQKTTELARVYSALASFMKVPFFDAGSVISTDGVDGIHFTEANNRDLGVALAEQVRSLL

SEQ ID NO: 315 - protein, 272 aa, Pseudomonas fluorescens
MSTFVAKDGTQIYFKDWGSGKPVLFSHGWPLDADMWEYQMEYLSSRGYRTIAFDRRGFGRSDQPWTGNDYDTFADDIAQLIEHLDLKEVTLVGFSMGGGDVARYIARHGSARVAGLVLLGAVTPLFGQKPDYPQGVPLDVFARFKTELLKDRAQFISDFNAPFYGINKGQVVSQGVQTQTLQIALLASLKATVDCVTAFAETDFRPDMAKIDVPTLVIHGDGDQIVPFETTGKVAAELIKGAELKVYKDAPHGFAVTHAQQLNEDLLAFLKR

SEQ ID NO: 316 - DNA, 1278 bp, artificial sequence
  synthetic construct
atgcagctgttcgatttgagcctggaagaattgaaaaagtacaaaccgaaaaagacggcgcgtccggacttttctgatttttggaaaaagtctctggaggaactgcgtcaggtcgaggcggagccgaccctggaaagctacgactaccctgtcaagggcgttaaggtgtaccgcctgacctaccagagcttcggtcatagcaaaatcgagggtttctatgcggtgccggaccaaaccggtccgcacccggcactggttcgtttccacggttataacgccagctatgatggcggtatccatgacatcgtcaattgggcactgcatggttacgcaacgtttggcatgctggttcgcggccaaggcggtagcgaggataccagcgttaccccgggtggccacgcgctgggctggatgaccaagggtattctgtccaaggatacctattactaccgtggtgtatacttggatgcagttcgtgcgctggaggtcattcaaagctttccggaagttgacgagcatcgtatcggtgtgattggtggtagccagggtggcgcgctggcgattgcagctgccgcgttgagcgatattccgaaagtcgtggttgcggactatccgtatctgtcgaactttgagcgcgctgtcgacgtggcactggaacaaccgtacctggagattaacagctacttccgccgtaatagcgacccgaaagtggaggagaaggcgtttgaaactctgagctattttgatctgatcaatctggcgggttgggtgaaacaaccgaccctgatggccattggcctgatcgataagatcactccgccgtctacggtgttcgcggcatataaccacctggaaaccgacaaagacttgaaagtttaccgttatttcggccacgagtttatcccagccttccagacggaaaagttgagcttcctgcagaaacacctgctgctgagcacgggtccgggcagcggcggtgctggttcccctggcagcgccggtggtccaggatcccctagcgcacaaagccaactgccggacaagcatagcggcctgcacgaacgtgctccgcagcgttacggtccggaaccggaaccggaaccggagccgatcccagaaccgccgaaagaggccccagttgttattgaaaagccgaagccgaaaccgaagccgaagccgaagccgcctgcgcatgatcataagaatcagaaggaaacccatcagcgtcacgccgctggttcgggcggtggtggtagcccgcaccatcaccaccaccac

SEQ ID NO: 317 - protein, 426 aa, artificial sequence
  synthetic construct
MQLFDLSLEELKKYKPKKTARPDFSDFWKKSLEELRQVEAEPTLESYDYPVKGVKVYRLTYQSFGHSKIEGFYAVPDQTGPHPALVRFHGYNASYDGGIHDIVNWALHGYATFGMLVRGQGGSEDTSVTPGGHALGWMTKGILSKDTYYYRGVYLDAVRALEVIQSFPEVDEHRIGVIGGSQGGALAIAAAALSDIPKVVVADYPYLSNFERAVDVALEQPYLEINSYFRRNSDPKVEEKAFETLSYFDLINLAGWVKQPTLMAIGLIDKITPPSTVFAAYNHLETDKDLKVYRYFGHEFIPAFQTEKLSFLQKHLLLSTGPGSGGAGSPGSAGGPGSPSAQSQLPDKHSGLHERAPQRYGPEPEPEPEPIPEPPKEAPVVIEKPKPKPKPKPKPPAHDHKNQKETHQRHAAGSGGGGSPHHHHHH

SEQ ID NO: 318 - DNA, 1254 bp, artificial sequence
  synthetic construct
atgaccaaaatcaacaattggcaagactaccaaggtagctctctgaaaccggaggacttcgataagttttgggacgagaaaatcaatctggtgagcaatcatcagtttgagtttgagctgatcgaaaagaacctgagcagcaaggttgtgaatttctatcatctgtggttcaccgcaattgacggtgcaaaaatccacgcgcaattgatcgtcccgaaaaacctgaaagaaaagtatcctgccatcctgcaatttcacggttatcactgcgatagcggcgactgggttgacaaaattggcatcgtggcggaaggcaacgtagtgctggcactggattgtcgcggtcagggtggcctgagccaagacaatatccagacgatgggtatgactatgaaaggtctgattgttcgcggcattgacgagggttatgagaacctgtactacgtccgtcaattcatggatctgatcaccgcgacgaagattctgagcgaattcgattttgtcgatgaaaccaacatcagcgcgcagggcgccagccaaggtggtgcgctggcggttgcgtgcgcggcactgagcccgctgattaagaaggtcacggctacgtacccgttcttgtccgactaccgtaaagcgtacgaactgggtgccgaggaaagcgcctttgaggagctgccatattggttccagtttaaagacccgttgcacttgcgtgaggattggttcttcaaccagctggaatacatcgacattcagaatctggctccgcgtattaaggcagaggttatttggatcttgggcggtaaagataccgtggtgccgccgattacccaaatggctgcgtacaacaagattcagtccaagaaaagcctgtatgttctgcctgaatacggccacgagtatctgccgaagatttcggattggctgcgcgaaaatcagggtccgggtagcggcggtgcgggttctccgggcagcgcaggcggtccgggatcccctagcgcacaaagccaactgccggacaagcatagcggcctgcacgaacgtgctccgcagcgttacggtccggaaccggaaccggaaccggagccgatcccagaaccgccgaaagaggccccagttgttattgaaaagccgaagccgaaaccgaagccgaagccgaagccgcctgcgcatgatcataagaatcagaaggaaacccatcagcgtcacgccgctggttcgggcggtggtggtagcccgcaccatcaccaccaccac

SEQ ID NO: 319 - protein, 418 aa, artificial sequence
  synthetic construct
MTKINNWQDYQGSSLKPEDFDKFWDEKINLVSNHQFEFELIEKNLSSKVVNFYHLWFTAIDGAKIHAQLIVPKNLKEKYPAILQFHGYHCDSGDWVDKIGIVAEGNVVLALDCRGQGGLSQDNIQTMGMTMKGLIVRGIDEGYENLYYVRQFMDLITATKILSEFDFVDETNISAQGASQGGALAVACAALSPLIKKVTATYPFLSDYRKAYELGAEESAFEELPYWFQFKDPLHLREDWFFNQLEYIDIQNLAPRIKAEVIWILGGKDTVVPPITQMAAYNKIQSKKSLYVLPEYGHEYLPKISDWLRENQGPGSGGAGSPGSAGGPGSPSAQSQLPDKHSGLHERAPQRYGPEPEPEPEPIPEPPKEAPVVIEKPKPKPKPKPKPPAHDHKNQKETHQRHAAGSGGGGSPHHHHHH

SEQ ID NO: 320 - DNA, 1287 bp, artificial sequence
  synthetic construct
atgccgtttccggatctgatccagccggagctgggcgcatacgtcagctccgtcggtatgccggacgatttcgctcaattctggaccagcaccattgccgaagctcgtcaggcaggtggcgaggttagcatcgtccaagctcagacgactctgaaagcagtccagagcttcgacgttaccttcccgggctacggcggtcacccgatcaagggttggctgatcctgccaacccaccacaaaggtcgcctgccgctggtggtacagtacattggttatggcggtggtcgtggcttggcgcatgaacagttgcactgggcagcatccggctttgcgtacttccgcatggacacccgtggtcaaggtagcgattggtcggttggtgagactgccgacccggttggtagcaccagcagcatcccgggctttatgacccgtggtgtgctggataagaatgactactattaccgtcgcttgttcacggacgcggtccgtgctattgatgcgctgctgggtctggactttgtggacccggagcgcattgccgtctgcggtgacagccagggtggcggtatcagcctggcggttggcggcatcgatccgcgtgttaaagcggttatgccggatgtgccgttcctgtgtgattttccgcgtgccgtccagacggccgttcgcgacccgtacctggagattgtgcgctttttggcacaacaccgtgaaaagaaagcagcggtgttcgaaaccctgaactattttgactgtgtgaattttgcgcgtcgtagcaaagcgcctgcgctgtttagcgtggcgctgatggatgaagtgtgcccgccatctaccgtttatggtgccttcaacgcgtatgcgggcgaaaagaccattacggagtacgagttcaataaccacgagggtggccaaggctatcaagaacgtcagcaaatgacgtggctgtctcgcctgttcggtgtcggcggtccgggtagcggtggtgcgggcagccctggcagcgcaggtggtccgggatcccctagcgcacaaagccaactgccggacaagcatagcggcctgcacgaacgtgctccgcagcgttacggtccggaaccggaaccggaaccggagccgatcccagaaccgccgaaagaggccccagttgttattgaaaagccgaagccgaaaccgaagccgaagccgaagccgcctgcgcatgatcataagaatcagaaggaaacccatcagcgtcacgccgctggttcgggcggtggtggtagcccgcaccatcaccaccaccac

SEQ ID NO: 321 - protein, 429 aa, artificial sequence
  synthetic construct
MPFPDLIQPELGAYVSSVGMPDDFAQFWTSTIAEARQAGGEVSIVQAQTTLKAVQSFDVTFPGYGGHPIKGWLILPTHHKGRLPLVVQYIGYGGGRGLAHEQLHWAASGFAYFRMDTRGQGSDWSVGETADPVGSTSSIPGFMTRGVLDKNDYYYRRLFTDAVRAIDALLGLDFVDPERIAVCGDSQGGGISLAVGGIDPRVKAVMPDVPFLCDFPRAVQTAVRDPYLEIVRFLAQHREKKAAVFETLNYFDCVNFARRSKAPALFSVALMDEVCPPSTVYGAFNAYAGEKTITEYEFNNHEGGQGYQERQQMTWLSRLFGVGGPGSGGAGSPGSAGGPGSPSAQSQLPDKHSGLHERAPQRYGPEPEPEPEPIPEPPKEAPVVIEKPKPKPKPKPKPPAHDHKNQKETHQRHAAGSGGGGSPHHHHHH

SEQ ID NO: 322 - DNA, 966 bp, artificial sequence
  synthetic construct
atggcgaaacgcattctgtgcttcggcgacagcctgacctggggttgggttccggtcgaggatggcgcaccgacggaacgttttgcgccggatgtgcgttggacgggtgtgctggctcagcaactgggtgccgattttgaggtcatcgaagagggtctggtcgcacgtacgaccaacattgatgacccgaccgacccgcgtctgaacggcgcaagctatttgccgagctgtctggcgacccacctgccgctggatctggtgattatcatgttgggcaccaatgataccaaagcttatttccgccgcaccccgctggacatcgcgctgggcatgagcgtcttggtgacgcaggttctgactagcgctggcggtgtcggtactacgtaccctgcgccgaaagtcctggtggttagcccgccaccgctggcgccgatgccgcacccgtggttccaactgatttttgaaggcggtgagcaaaagacgaccgagttggcccgtgtttacagcgcgttggcgagctttatgaaagttccgtttttcgacgcgggcagcgttattagcaccgatggcgtggacggtatccatttcaccgaagcaaataaccgtgacctgggtgtggccctggctgaacaagtgcgcagcctgctgggtccgggctccggtggtgccggttcgccgggtagcgcaggcggtcctggatccccttccgcgcagagccagctgccggacaaacactccggtctgcacgagcgcgcaccgcagcgttacggtccggagccagagccggaaccggagccgatcccggagccgccgaaagaagcgccggttgttatcgagaagccgaaaccgaagccaaagcctaagccgaagccaccggcccatgaccacaaaaatcaaaaggaaacccatcagcgtcacgcagcgggttctggtggtggcggcagcccgcatcaccaccatcaccat

SEQ ID NO: 323 - protein, 322 aa, artificial sequence
  synthetic construct
MAKRILCFGDSLTWGWVPVEDGAPTERFAPDVRWTGVLAQQLGADFEVIEEGLVARTTNIDDPTDPRLNGASYLPSCLATHLPLDLVIIMLGTNDTKAYFRRTPLDIALGMSVLVTQVLTSAGGVGTTYPAPKVLVVSPPPLAPMPHPWFQLIFEGGEQKTTELARVYSALASFMKVPFFDAGSVISTDGVDGIHFTEANNRDLGVALAEQVRSLLGPGSGGAGSPGSAGGPGSPSAQSQLPDKHSGLHERAPQRYGPEPEPEPEPIPEPPKEAPVVIEKPKPKPKPKPKPPAHDHKNQKETHQRHAAGSGGGGSPHHHHHH

SEQ ID NO: 324 - DNA, 966 bp, artificial sequence
  synthetic construct
atggcgaaacgcattctgtgcttcggcgacagcctgacctggggttgggttccggtcgaggatggcgcaccgacggaacgttttgcgccggatgtgcgttggacgggtgtgctggctcagcaactgggtgccgattttgaggtcatcgaagagggtctggtcgcacgtacgaccaacattgatgacccgaccgacccgcgtctgaacggcgcaagctatttgccgagctgtctggcgacccacctgccgctggatctggtgattatcatgttgggcaccaatgataccaaagcttatttccgccgcaccccgctggacatcgcgctgggcatgagcgtcttggtgacgcaggttctgactagcgctggcggtgtcggtactacgtaccctgcgccgaaagtcctggtggttagcccgccaccgctggcgccgatgccgcacccgtggttccaactgatttttgaaggcggtgagcaaaagacgaccgagttggcccgtgtttacagcgcgttggcgagctttatgaaagttccgtttttcgacgcgggcagcgttattagcaccgatggcgtggacggtatccatttcaccgaagcaaataaccgtgacctgggtgtggccctggctgaacaagtgcgcagcctgctgggtccgggctccggtggtgccggttcgccgggtagcgcaggcggtcctggatccccgagcgcgcaatcccagttgccggatcgccacagcggtctgcatgagcgtgccccgcaacgttacggtccagagccggagccggaaccggagccgattccggaaccaccgcgcgaggctccggtggttatcgaacgtccacgtccacgcccgcgcccgcgtccgcgtccaccggcgcacgaccaccgtaatcaacgtgaaacccaccagcgccacgcagccggcagcggtggtggtggtagcccgcatcatcaccatcatcac

SEQ ID NO: 325 - protein, 322 aa, artificial sequence
  synthetic construct
MAKRILCFGDSLTWGWVPVEDGAPTERFAPDVRWTGVLAQQLGADFEVIEEGLVARTTNIDDPTDPRLNGASYLPSCLATHLPLDLVIIMLGTNDTKAYFRRTPLDIALGMSVLVTQVLTSAGGVGTTYPAPKVLVVSPPPLAPMPHPWFQLIFEGGEQKTTELARVYSALASFMKVPFFDAGSVISTDGVDGIHFTEANNRDLGVALAEQVRSLLGPGSGGAGSPGSAGGPGSPSAQSQLPDRHSGLHERAPQRYGPEPEPEPEPIPEPPREAPVVIERPRPRPRPRPRPPAHDHRNQRETHQRHAAGSGGGGSPHHHHHH

SEQ ID NO: 326 - DNA, 978 bp, artificial sequence
  synthetic construct
atggcgaaacgcattctgtgcttcggcgacagcctgacctggggttgggttccggtcgaggatggcgcaccgacggaacgttttgcgccggatgtgcgttggacgggtgtgctggctcagcaactgggtgccgattttgaggtcatcgaagagggtctggtcgcacgtacgaccaacattgatgacccgaccgacccgcgtctgaacggcgcaagctatttgccgagctgtctggcgacccacctgccgctggatctggtgattatcatgttgggcaccaatgataccaaagcttatttccgccgcaccccgctggacatcgcgctgggcatgagcgtcttggtgacgcaggttctgactagcgctggcggtgtcggtactacgtaccctgcgccgaaagtcctggtggttagcccgccaccgctggcgccgatgccgcacccgtggttccaactgatttttgaaggcggtgagcaaaagacgaccgagttggcccgtgtttacagcgcgttggcgagctttatgaaagttccgtttttcgacgcgggcagcgttattagcaccgatggcgtggacggtatccatttcaccgaagcaaataaccgtgacctgggtgtggccctggctgaacaagtgcgcagcctgctgggtccgggctccggtggtgccggttcgccgggtagcgcaggcggtcctggatccgcgcaaagccaactgccggacaaacatagcggtctgcacgagcgtgcgccgcagcgttacggtagcggtacggcggaaattcaatccagcaagaacccgaacccgcacccgcagcgcagctggaccaatggcagcggtcataatcacatgcaagagcgttacacggacccgcagcacagcccgagcgttaatggtttgggtagcggccacgaccataagaatcagaaagaaacccatcaacgccacgcggcgtccagccaccaccaccatcaccac

SEQ ID NO: 327 - protein, 326 aa, artificial sequence
  synthetic construct
MAKRILCFGDSLTWGWVPVEDGAPTERFAPDVRWTGVLAQQLGADFEVIEEGLVARTTNIDDPTDPRLNGASYLPSCLATHLPLDLVIIMLGTNDTKAYFRRTPLDIALGMSVLVTQVLTSAGGVGTTYPAPKVLVVSPPPLAPMPHPWFQLIFEGGEQKTTELARVYSALASFMKVPFFDAGSVISTDGVDGIHFTEANNRDLGVALAEQVRSLLGPGSGGAGSPGSAGGPGSAQSQLPDKHSGLHERAPQRYGSGTAEIQSSKNPNPHPQRSWTNGSGHNHMQERYTDPQHSPSVNGLGSGHDHKNQKETHQRHAASSHHHHHH

SEQ ID NO: 328 - DNA, 750 bp, artificial sequence
  synthetic construct
atggcgaaacgcattctgtgcttcggcgacagcctgacctggggttgggttccggtcgaggatggcgcaccgacggaacgttttgcgccggatgtgcgttggacgggtgtgctggctcagcaactgggtgccgattttgaggtcatcgaagagggtctggtcgcacgtacgaccaacattgatgacccgaccgacccgcgtctgaacggcgcaagctatttgccgagctgtctggcgacccacctgccgctggatctggtgattatcatgttgggcaccaatgataccaaagcttatttccgccgcaccccgctggacatcgcgctgggcatgagcgtcttggtgacgcaggttctgactagcgctggcggtgtcggtactacgtaccctgcgccgaaagtcctggtggttagcccgccaccgctggcgccgatgccgcacccgtggttccaactgatttttgaaggcggtgagcaaaagacgaccgagttggcccgtgtttacagcgcgttggcgagctttatgaaagttccgtttttcgacgcgggcagcgttattagcaccgatggcgtggacggtatccatttcaccgaagcaaataaccgtgacctgggtgtggccctggctgaacaagtgcgcagcctgctgggtccgggctccggtggtgccggttcgccgggtagcgcaggcggtcctggatccggcaaaggcaagggtaagggtaaaggtaaacatcatcaccaccaccac

SEQ ID NO: 329 - protein, 250 aa, artificial sequence
  synthetic construct
MAKRILCFGDSLTWGWVPVEDGAPTERFAPDVRWTGVLAQQLGADFEVIEEGLVARTTNIDDPTDPRLNGASYLPSCLATHLPLDLVIIMLGTNDTKAYFRRTPLDIALGMSVLVTQVLTSAGGVGTTYPAPKVLVVSPPPLAPMPHPWFQLIFEGGEQKTTELARVYSALASFMKVPFFDAGSVISTDGVDGIHFTEANNRDLGVALAEQVRSLLGPGSGGAGSPGSAGGPGSGKGKGKGKGKHHHHHH

SEQ ID NO: 330 - DNA, 1134 bp, artificial sequence
  synthetic construct
atgtccaccttcgttgcgaaagatggcacccagatttactttaaagactggggcagcggcaagccggttctgtttagccacggctggccgctggacgcggatatgtgggagtatcagatggagtacctgagcagccgtggttaccgtaccatcgccttcgatcgccgtggttttggtcgcagcgatcaaccgtggaccggcaatgattatgacacgttcgcagatgacattgcccagctgatcgagcacctggacctgaaagaggttaccctggtcggtttcagcatgggcggtggtgacgtcgcgcgctacattgcgcgtcatggttccgctcgtgtggcgggtctggtcctgctgggtgctgtaacgccactgtttggtcaaaagccggattatccgcagggtgtgccgttggatgtgtttgcgcgcttcaaaaccgagttgctgaaagaccgtgcgcaattcatcagcgacttcaacgcaccgttttacggtatcaacaaaggccaagttgtcagccagggcgttcaaacgcagacgctgcagattgcgctgctggcaagcctgaaggcgaccgttgactgcgtgacggcttttgcggaaactgattttcgtccggacatggcgaagattgatgttccgaccttggtgattcacggtgacggcgatcagatcgtgccgttcgaaaccaccggtaaggttgcggccgagctgatcaaaggtgcggagctgaaagtgtacaaggacgcgcctcacggcttcgcagtcactcatgcacagcaactgaacgaggacttgctggccttcttgaaacgcggtccgggctccggtggcgcaggcagcccgggtagcgcaggtggtccgggatccccgagcgcccaaagccagctgcctgataagcacagcggtctgcatgagcgtgctcctcaacgttatggtccggagccggaaccggaaccagagccgatcccggaaccgccgaaggaagccccggtcgtgattgaaaaaccgaaaccgaagccgaaaccaaagccgaagccgccagcgcatgaccacaagaatcagaaagaaacgcaccaacgtcacgccgctggctctggcggtggcggttcgccgcatcatcaccaccaccat

SEQ ID NO: 331 - protein, 378 aa, artificial sequence
  synthetic construct
MSTFVAKDGTQIYFKDWGSGKPVLFSHGWPLDADMWEYQMEYLSSRGYRTIAFDRRGFGRSDQPWTGNDYDTFADDIAQLIEHLDLKEVTLVGFSMGGGDVARYIARHGSARVAGLVLLGAVTPLFGQKPDYPQGVPLDVFARFKTELLKDRAQFISDFNAPFYGINKGQVVSQGVQTQTLQIALLASLKATVDCVTAFAETDFRPDMAKIDVPTLVIHGDGDQIVPFETTGKVAAELIKGAELKVYKDAPHGFAVTHAQQLNEDLLAFLKRGPGSGGAGSPGSAGGPGSPSAQSQLPDKHSGLHERAPQRYGPEPEPEPEPIPEPPKEAPVVIEKPKPKPKPKPKPPAHDHKNQKETHQRHAAGSGGGGSPHHHHHH

SEQ ID NO: 332 - DNA, 1134 bp, artificial sequence
  synthetic construct
atgtccaccttcgttgcgaaagatggcacccagatttactttaaagactggggcagcggcaagccggttctgtttagccacggctggccgctggacgcggatatgtgggagtatcagatggagtacctgagcagccgtggttaccgtaccatcgccttcgatcgccgtggttttggtcgcagcgatcaaccgtggaccggcaatgattatgacacgttcgcagatgacattgcccagctgatcgagcacctggacctgaaagaggttaccctggtcggtttcagcatgggcggtggtgacgtcgcgcgctacattgcgcgtcatggttccgctcgtgtggcgggtctggtcctgctgggtgctgtaacgccactgtttggtcaaaagccggattatccgcagggtgtgccgttggatgtgtttgcgcgcttcaaaaccgagttgctgaaagaccgtgcgcaattcatcagcgacttcaacgcaccgttttacggtatcaacaaaggccaagttgtcagccagggcgttcaaacgcagacgctgcagattgcgctgctggcaagcctgaaggcgaccgttgactgcgtgacggcttttgcggaaactgattttcgtccggacatggcgaagattgatgttccgaccttggtgattcacggtgacggcgatcagatcgtgccgttcgaaaccaccggtaaggttgcggccgagctgatcaaaggtgcggagctgaaagtgtacaaggacgcgcctcacggcttcgcagtcactcatgcacagcaactgaacgaggacttgctggccttcttgaaacgcggtccgggctccggtggcgcaggcagcccgggtagcgcaggtggtccgggatccccgagcgcgcaatcccagttgccggatcgccacagcggtctgcatgagcgtgccccgcaacgttacggtccagagccggagccggaaccggagccgattccggaaccaccgcgcgaggctccggtggttatcgaacgtccacgtccacgcccgcgcccgcgtccgcgtccaccggcgcacgaccaccgtaatcaacgtgaaacccaccagcgccacgcagccggcagcggtggtggtggtagcccgcatcatcaccatcatcac

SEQ ID NO: 333 - protein, 378 aa, artificial sequence
  synthetic construct
MSTFVAKDGTQIYFKDWGSGKPVLFSHGWPLDADMWEYQMEYLSSRGYRTIAFDRRGFGRSDQPWTGNDYDTFADDIAQLIEHLDLKEVTLVGFSMGGGDVARYIARHGSARVAGLVLLGAVTPLFGQKPDYPQGVPLDVFARFKTELLKDRAQFISDFNAPFYGINKGQVVSQGVQTQTLQIALLASLKATVDCVTAFAETDFRPDMAKIDVPTLVIHGDGDQIVPFETTGKVAAELIKGAELKVYKDAPHGFAVTHAQQLNEDLLAFLKRGPGSGGAGSPGSAGGPGSPSAQSQLPDRHSGLHERAPQRYGPEPEPEPEPIPEPPREAPVVIERPRPRPRPRPRPPAHDHRNQRETHQRHAAGSGGGGSPHHHHHH

SEQ ID NO: 334 - DNA, 1146 bp, artificial sequence
  synthetic construct
atgtccaccttcgttgcgaaagatggcacccagatttactttaaagactggggcagcggcaagccggttctgtttagccacggctggccgctggacgcggatatgtgggagtatcagatggagtacctgagcagccgtggttaccgtaccatcgccttcgatcgccgtggttttggtcgcagcgatcaaccgtggaccggcaatgattatgacacgttcgcagatgacattgcccagctgatcgagcacctggacctgaaagaggttaccctggtcggtttcagcatgggcggtggtgacgtcgcgcgctacattgcgcgtcatggttccgctcgtgtggcgggtctggtcctgctgggtgctgtaacgccactgtttggtcaaaagccggattatccgcagggtgtgccgttggatgtgtttgcgcgcttcaaaaccgagttgctgaaagaccgtgcgcaattcatcagcgacttcaacgcaccgttttacggtatcaacaaaggccaagttgtcagccagggcgttcaaacgcagacgctgcagattgcgctgctggcaagcctgaaggcgaccgttgactgcgtgacggcttttgcggaaactgattttcgtccggacatggcgaagattgatgttccgaccttggtgattcacggtgacggcgatcagatcgtgccgttcgaaaccaccggtaaggttgcggccgagctgatcaaaggtgcggagctgaaagtgtacaaggacgcgcctcacggcttcgcagtcactcatgcacagcaactgaacgaggacttgctggccttcttgaaacgcggtccgggctccggtggcgcaggcagcccgggtagcgcaggtggtccgggatccgcgcaaagccaactgccggacaaacatagcggtctgcacgagcgtgcgccgcagcgttacggtagcggtacggcggaaattcaatccagcaagaacccgaacccgcacccgcagcgcagctggaccaatggcagcggtcataatcacatgcaagagcgttacacggacccgcagcacagcccgagcgttaatggtttgggtagcggccacgaccataagaatcagaaagaaacccatcaacgccacgcggcgtccagccaccaccaccatcaccac

SEQ ID NO: 335 - protein, 382 aa, artificial sequence
  synthetic construct
MSTFVAKDGTQIYFKDWGSGKPVLFSHGWPLDADMWEYQMEYLSSRGYRTIAFDRRGFGRSDQPWTGNDYDTFADDIAQLIEHLDLKEVTLVGFSMGGGDVARYIARHGSARVAGLVLLGAVTPLFGQKPDYPQGVPLDVFARFKTELLKDRAQFISDFNAPFYGINKGQVVSQGVQTQTLQIALLASLKATVDCVTAFAETDFRPDMAKIDVPTLVIHGDGDQIVPFETTGKVAAELIKGAELKVYKDAPHGFAVTHAQQLNEDLLAFLKRGPGSGGAGSPGSAGGPGSAQSQLPDKHSGLHERAPQRYGSGTAEIQSSKNPNPHPQRSWTNGSGHNHMQERYTDPQHSPSVNGLGSGHDHKNQKETHQRHAASSHHHHHH

SEQ ID NO: 336 - DNA, 918 bp, artificial sequence
  synthetic construct
atgtccaccttcgttgcgaaagatggcacccagatttactttaaagactggggcagcggcaagccggttctgtttagccacggctggccgctggacgcggatatgtgggagtatcagatggagtacctgagcagccgtggttaccgtaccatcgccttcgatcgccgtggttttggtcgcagcgatcaaccgtggaccggcaatgattatgacacgttcgcagatgacattgcccagctgatcgagcacctggacctgaaagaggttaccctggtcggtttcagcatgggcggtggtgacgtcgcgcgctacattgcgcgtcatggttccgctcgtgtggcgggtctggtcctgctgggtgctgtaacgccactgtttggtcaaaagccggattatccgcagggtgtgccgttggatgtgtttgcgcgcttcaaaaccgagttgctgaaagaccgtgcgcaattcatcagcgacttcaacgcaccgttttacggtatcaacaaaggccaagttgtcagccagggcgttcaaacgcagacgctgcagattgcgctgctggcaagcctgaaggcgaccgttgactgcgtgacggcttttgcggaaactgattttcgtccggacatggcgaagattgatgttccgaccttggtgattcacggtgacggcgatcagatcgtgccgttcgaaaccaccggtaaggttgcggccgagctgatcaaaggtgcggagctgaaagtgtacaaggacgcgcctcacggcttcgcagtcactcatgcacagcaactgaacgaggacttgctggccttcttgaaacgcggtccgggctccggtggcgcaggcagcccgggtagcgcaggtggtccgggatccggcaaaggcaagggtaagggtaaaggtaaacatcatcaccaccaccac

SEQ ID NO: 337 - protein, 306 aa, artificial sequence
  synthetic construct
MSTFVAKDGTQIYFKDWGSGKPVLFSHGWPLDADMWEYQMEYLSSRGYRTIAFDRRGFGRSDQPWTGNDYDTFADDIAQLIEHLDLKEVTLVGFSMGGGDVARYIARHGSARVAGLVLLGAVTPLFGQKPDYPQGVPLDVFARFKTELLKDRAQFISDFNAPFYGINKGQVVSQGVQTQTLQIALLASLKATVDCVTAFAETDFRPDMAKIDVPTLVIHGDGDQIVPFETTGKVAAELIKGAELKVYKDAPHGFAVTHAQQLNEDLLAFLKRGPGSGGAGSPGSAGGPGSGKGKGKGKGKHHHHHH

SEQ ID NO: 338 - protein, 216 aa, Mycobacterium smegmatis
MAKRILCFGDSLTWGWVPVEDGAPTERFAPDVRWTGVLAQQLGADFEVIEEGLSARTTNIDDPTDPRLNGASYLPSCLATHLPLDLVIIMLGTNDTKAYFRRTPLDIALGMSVLVTQVLTSAGGVGTTYPAPKVLVVSPPPLAPMPHPWFQLIFEGGEQKTTELARVYSALASFMKVPFFDAGSVISTDGVDGIHFTEANNRDLGVALAEQVRSLL

SEQ ID NO: 339 - protein, 272 aa, Pseudomonas fluorescens
MSTFVAKDGTQIYFKDWGSGKPVLFSHGWLLDADMWEYQMEYLSSRGYRTIAFDRRGFGRSDQPWTGNDYDTFADDIAQLIEHLDLKEVTLVGFSMGGGDVARYIARHGSARVAGLVLLGAVTPLFGQKPDYPQGVPLDVFARFKTELLKDRAQFISDFNAPFYGINKGQVVSQGVQTQTLQIALLASLKATVDCVTAFAETDFRPDMAKIDVPTLVIHGDGDQIVPFETTGKVAAELIKGAELKVYKDAPHGFAVTHAQQLNEDLLAFLKR

(19) United States
(12) Patent Application Publication
(10) Pub. No.: US 2012/0317732 A1
(43) Pub. Date: Dec. 20, 2012

(54) NON-AQUEOUS STABLE COMPOSITION FOR DELIVERING SUBSTRATES FOR A DEPILATORY PRODUCT USING PERACIDS

(75) Inventors: Xueping Jiang, Wilmington, DE (US); Tanja Maria Gruber, Media, PA (US); Pierre E. Rouviere, Wilmington, DE (US)

(73) Assignee: E. I. DU PONT DE NEMOURS AND COMPANY, WILMINGTON, DE (US)

(21) Appl. No.: 13/329,951

(22) Filed: Dec. 19, 2011

Related U.S. Application Data
(60) Provisional application No. 61/424,847, filed on Dec. 20, 2010.

(51) Int. Cl. A61K 8/66; A61Q 9/04; B65D 85/00; B65D 25/04; B65D 1/04; A61K 8/92; A61K 8/73

(52) U.S. Cl. 8/161; 220/500; 215/6; 206/524.1

(57) ABSTRACT

Disclosed herein are compositions and methods for delivering substrates for a depilatory product using an enzymatically generated peracid. More specifically, a two component system is provided comprising (a) a first non-aqueous composition comprising a solid source of peroxygen, an ester substrate, and an optional organic cosolvent and (b) an aqueous component having a pH of at least 4 comprising an enzyme catalyst having perhydrolytic activity and a buffer. The perhydrolytic enzyme catalyst may be in the form of a fusion protein comprising a perhydrolytic enzyme coupled through an optional peptide linker to a peptidic component having affinity for hair.

### CROSS-REFERENCE TO RELATED APPLICATIONS

This application claims benefit of U.S. Provisional Patent Application No. 61/424,847 filed Dec. 20, 2010, which is incorporated by reference herein in its entirety.

### FIELD OF THE INVENTION

This invention relates to the field of personal care products comprising at least one enzymatically produced peracid as hair care benefit agent. Specifically, a hair care product comprising a two component peracid generation system is provided wherein the first component is a non-aqueous composition comprising a carboxylic acid ester and a solid source of peroxygen and the second component is an aqueous composition comprising an enzyme having perhydrolytic activity. The two components are combined to generate the peracid benefit agent. The perhydrolytic enzyme may be in the form a fusion protein engineered to contain at least one peptidic component having affinity for hair.

### BACKGROUND OF THE INVENTION

Peroxycarboxylic acids (“peracids”) are effective antimicrobial agents. Methods to clean, disinfect, and/or sanitize hard surfaces, food products, living plant tissues, and medical devices against undesirable microbial growth have been described (e.g., U.S. Pat. No. 6,545,047; U.S. Pat. No. 6,183,807; U.S. Pat. No. 6,518,307; U.S. Pat. No. 5,683,724; and U.S. Patent Application Publication No. 2003-0026846 A1). Peracids have also been reported to be useful in preparing bleaching compositions for laundry detergent applications (e.g., U.S. Pat. No. 3,974,082; U.S. Pat. No. 5,296,161; and U.S. Pat. No. 5,364,554).

It has also been reported that peracids may oxidize keratinous materials such as hair, skin and nails. For example, United Kingdom published patent specification GB 692,478(A) to Alexander, P., et al. describes a method of oxidizing the disulfide bonds of keratinous materials to sulphydryl or sulphonic acids using an aqueous solution of saturated peraliphatic acids not having more than 4 carbon atoms at a temperature below 100° C., such that the oxidized material is readily soluble in dilute alkali. Lillie et al. (J. Histochem. Cytochem., (1954) 95-102) discloses oxidation-induced basophilia of keratinous structures. U.S. Pat. No. 6,270,791 to Van Dyke et al. discloses a method to obtain water soluble peptides from a keratin-containing source, such as hair, comprising oxidizing a keratin-containing material in an aqueous solution for form water soluble peptides. The oxidizing agent may include peracetic acid.

Hair care compositions and methods describing the use of a peracid have been reported. Chinese Patent Application Publication CN101440575 A to Zheng, Y., discloses a method of treating hair with peracetic acid and a catalase followed by treating hair with a protease. US2002-0053110 A1; U.S. Pat. No. 6,022,381; U.S. Pat. No. 6,004,355; WO97/24106; and WO97/24108 to Dias et al. describe hair coloring compositions comprising a peroxyacid oxidizing agent and an oxidative hair coloring agent. U.S. Pat. No. 3,679,347 to Brown, F., describes dyeing human hair with a peroxy compound and a reactive dyestuff. United Kingdom patent GB1560399 A to Clark et al. describes compositions for hair treatment comprising an organic peracid component and an aqueous foam-forming solution containing an organic surfactant and a C10-C21 fatty acid amide. German patent application publication DE19733841 A1 to Till et al. discloses an agent for oxidative treatment of human hair comprising magnesium monoperphthalate.

Hahn, F. et al. (Leder (1967) 18(8):184-192) discloses a method of unhairing by oxidizing hair keratin with peracetic acid, Na₂O₂, and CARCAT® or ClO₂; followed by dissolving the oxidized hair with alkali. U.S. Pat. No. 3,479,127 to Hahn et al. discloses a process for unhairing of skins (calfskins, goatskins, sheepskin) and cowhides with peracids (3 hour treatment of 0.5 to 5 wt % peracetic acid, pH 2 to 5.5) followed by treatment with neutral salts or weak or strong alkaline acting salts or bases.

The inclusion of specific variant subtilisin Carlsberg proteases having perhydrolytic activity in a body care product is disclosed in U.S. Pat. No. 7,510,859 to Wieland et al. Perhydrolytic enzymes beyond the specific variant proteases are not described nor are there any working examples demonstrating the enzymatic production of peracid as a personal care benefit agent.

U.S. Patent Application Publication Nos. 2008-0176783 A1; 2008-0176299 A1; 2009-0005590 A1; 2010-0087529 A1; and 2010-0041752 A1 to DiCosimo et al. disclose enzymes structurally classified as members of the CE-7 family of carbohydrate esterases (i.e., cephalosporin C deacetylases [CAHs] and acetyl xylan esterases [AXEs]) that are characterized by significant perhydrolytic activity for converting carboxylic acid ester substrates into peroxycarboxylic acids at concentrations sufficient for use as a disinfectant and/or a bleaching agent.

Co-owned and copending patent application entitled “ENZYMATIC PERACID GENERATION FOR USE 1N HAIR CARE PRODUCTS” (attorney docket number CL5175) discloses the use of a peracid as a benefit agent in hair care products. The peracid-based benefit agent is used to provide a benefit such as hair removal, hair weakening, hair bleaching, hair styling, hair curling, hair conditioning, hair pretreating prior to application of a non-peracid-based benefit agent, and combinations thereof.

The reaction components when enzymatically generating peracids typically require (a) a perhydrolytic enzyme, (b) a suitable carboxylic acid ester, and (3) a source of peroxygen wherein one or more of the components remain separated until use. As such, multi-component generation systems are needed such that the reaction components are storage stable yet can quickly generate an efficacious concentration of peracid when combined under suitable reaction conditions. Some generation systems are designed such that the enzymatic component is stored in the substantially non-aqueous carboxylic acid ester and is then mixed with an aqueous component comprising hydrogen peroxide to generate the peracid. However, some hair care applications and products may require a generation system where the enzyme catalyst is not stored in the carboxylic acid ester substrate.

The problem to be solved is to provide an enzymatic generation system that is suitable with certain hair care applications, such as hair depilatory applications, and is storage stable for extended periods of time for both the enzyme catalyst and the substrates until use.

Peracids are strong oxidizing agents that may be reactive towards a variety of materials, including materials not targeted for the desired benefit. As such, certain personal care applications may benefit from the ability to target/focus the peracid benefit agent to the desired body surface by localizing peracid production on or near the desired target body surface. Enzymatic peracid production may benefit by targeting the perhydrolase to the body surface.

The use of shorter, biopanned peptides to target a cosmetic benefit agent to a body surface has been described (U.S. Pat. Nos. 7,220,405; 7,309,482; 7,285,264 and 7,807,141; U.S. Patent Application Publication Nos. 2005-0226839 A1; 2007-0196305 A1; 2006-0199206 A1; 2007-0065387 A1; 2008-0107614 A1; 2007-0110686 A1; 2006-0073111 A1; 2010-0158846; 2010-0158847; and 2010-0247589; and published PCT applications WO2008/054746; WO2004/048399, and WO2008/073368). The use of a peptidic material having affinity for hair to couple an active perhydrolytic enzyme (i.e., “targeted perhydrolases”) for the production of a peracid benefit agent has not been described.

As such, an additional problem to be solved is to provide storage stable hair care compositions that are compatible with targeted enzyme delivery systems.

### SUMMARY OF THE INVENTION

Hair care products and methods of use are provided to enzymatically produce a peracid benefit agent that may be used in applications such as hair removal (depilatory agent), a decrease in hair tensile strength, a hair pretreatment used to enhance other depilatory products (such as thioglycolate-based hair removal products), hair bleaching, hair dye pretreatment (oxidative hair dyes), hair curling, and hair conditioning.

The hair care products are comprised of a two component system comprising (1) a non-aqueous component comprising the carboxylic acid ester substrate, optionally diluted with an organic cosolvent, and a solid source of peroxygen, such as percarbonates or perborates, and (2) an aqueous composition comprising the perhydrolytic enzyme and a buffering agent; wherein the aqueous composition has a pH value of at least pH4 prior to combining the two components (i.e., during storage), whereby the desired peracid is generated by combining components (1) and (2).

### Brief Description of the Biological Sequences

The following sequences comply with 37 C.F.R. §§1.821-1.825 (“Requirements for patent applications Containing Nucleotide Sequences and/or Amino Acid Sequence Disclosures—the Sequence Rules”) and are consistent with World Intellectual Property Organization (WIPO) Standard ST.25 (2009) and the sequence listing requirements of the European Patent Convention (EPC) and the Patent Cooperation Treaty (PCT) Rules 5.2 and 49.5(a-bis), and Section 208 and Annex C of the Administrative Instructions. The symbols and format used for nucleotide and amino acid sequence data comply with the rules set forth in 37 C.F.R. §1.822.

SEQ ID NO: 1 is the nucleic acid sequence encoding a cephalosporin C deacetylase from Bacillus subtilis ATCC® 31954™.

SEQ ID NO: 2 is the amino acid sequence of a cephalosporin C deacetylase from Bacillus subtilis ATCC® 31954™.

SEQ ID NO: 3 is the nucleic acid sequence encoding a cephalosporin C deacetylase from Bacillus subtilis subsp. subtilis strain 168.

SEQ ID NO: 4 is the amino acid sequence of a cephalosporin C deacetylase from Bacillus subtilis subsp. subtilis strain 168.

SEQ ID NO: 5 is the nucleic acid sequence encoding a cephalosporin C deacetylase from B. subtilis ATCC® 6633™.

SEQ ID NO: 6 is the acid sequence of a cephalosporin C deacetylase from B. subtilis ATCC® 6633™.

SEQ ID NO: 7 is the nucleic acid sequence encoding a cephalosporin C deacetylase from B. lichenifoemis ATCC® 14580™.

SEQ ID NO: 8 is the deduced amino acid sequence of a cephalosporin C deacetylase from B. lichenifoemis ATCC® 14580™.

SEQ ID NO: 9 is the nucleic acid sequence encoding an acetyl xylan esterase from B. pumilus PS213.

SEQ ID NO: 10 is the deduced amino acid sequence of an acetyl xylan esterase from B. pumilus PS213.

SEQ ID NO: 11 is the nucleic acid sequence encoding an acetyl xylan esterase from Clostridium thermocellum ATCC® 27405™.

SEQ ID NO: 12 is the deduced amino acid sequence of an acetyl xylan esterase from Clostridium thermocellum ATCC® 27405™.

SEQ ID NO: 13 is the nucleic acid sequence encoding an acetyl xylan esterase from Thermotoga neapolitana.

SEQ ID NO: 14 is the amino acid sequence of an acetyl xylan esterase from Thermotoga neapolitana.

SEQ ID NO: 15 is the nucleic acid sequence encoding an acetyl xylan esterase from Thermotoga maritime MSB8.

SEQ ID NO: 16 is the amino acid sequence of an acetyl xylan esterase from Thermotoga maritime MSB8.

SEQ ID NO: 17 is the nucleic acid sequence encoding an acetyl xylan esterase from Thermoanaerobacterium sp. JW/SL YS485.

SEQ ID NO: 18 is the deduced amino acid sequence of an acetyl xylan esterase from Thermoanaerobacterium sp. JW/SL Y5485.

SEQ ID NO: 19 is the nucleic acid sequence of a cephalosporin C deacetylase from Bacillus sp. NRRL B-14911. It should be noted that the nucleic acid sequence encoding the cephalosporin C deacetylase from Bacillus sp. NRRL B-14911 as reported in GEN BANK® Accession number ZP_{—}01168674 appears to encode a 15 amino acid N-terminal addition that is likely incorrect based on sequence alignments with other cephalosporin C deacetylases and a comparison of the reported length (340 amino acids) versus the observed length of other CAH enzymes (typically 318-325 amino acids in length; see U.S. Patent Application Publication No. US-2010-0087528-A1; herein incorporated by reference). As such, the nucleic acid sequence as reported herein encodes the cephalosporin C deacetylase sequence from Bacillus sp. NRRL B-14911 without the N-terminal 15 amino acids reported under GENBANK® Accession number ZP_{—}01168674.

SEQ ID NO: 20 is the deduced amino acid sequence of the cephalosporin C deacetylase from Bacillus sp. NRRL B-14911 encoded by the nucleic acid sequence of SEQ ID NO: 19.

SEQ ID NO: 21 is the nucleic acid sequence encoding a cephalosporin C deacetylase from Bacillus halodurans C-125.

SEQ ID NO: 22 is the deduced amino acid sequence of a cephalosporin C deacetylase from Bacillus halodurans C-125.

SEQ ID NO: 23 is the nucleic acid sequence encoding a cephalosporin C deacetylase from Bacillus clausii KSM-K16.

SEQ ID NO: 24 is the deduced amino acid sequence of a cephalosporin C deacetylase from Bacillus clausii KSM-K16.

SEQ ID NO: 25 is the nucleic acid sequence encoding a Bacillus subtilis ATCC® 29233™ cephalosporin C deacetylase (CAH).

SEQ ID NO: 26 is the deduced amino acid sequence of a Bacillus subtilis ATCC® 29233™ cephalosporin C deacetylase (CAH).

SEQ ID NO: 27 is the deduced amino acid sequence of a Thermotoga neapolitana acetyl xylan esterase variant from U.S. Patent Application Publication No. 2010-0087529 (incorporated herein by reference in its entirety), where the Xaa residue at position 277 is Ala, Val, Ser, or Thr.

SEQ ID NO: 28 is the deduced amino acid sequence of a Thermotoga maritime MSB8 acetyl xylan esterase variant from U.S. Patent Application Publication No. 2010-0087529, where the Xaa residue at position 277 is Ala, Val, Ser, or Thr.

SEQ ID NO: 29 is the deduced amino acid sequence of a Thermotoga lettingae acetyl xylan esterase variant from U.S. Patent Application Publication No. 2010-0087529, where the Xaa residue at position 277 is Ala, Val, Ser, or Thr.

SEQ ID NO: 30 is the deduced amino acid sequence of a Thermotoga petrophila acetyl xylan esterase variant from U.S. Patent Application Publication No. 2010-0087529, where the Xaa residue at position 277 is Ala, Val, Ser, or Thr.

SEQ ID NO: 31 is the deduced amino acid sequence of a Thermotoga sp. RQ2 acetyl xylan esterase variant derived from“RQ2(a)” from U.S. Patent Application Publication No. 2010-0087529, where the Xaa residue at position 277 is Ala, Val, Ser, or Thr.

SEQ ID NO: 32 is the deduced amino acid sequence of a Thermotoga sp. RQ2 acetyl xylan esterase variant derived from “RQ2(b)” from U.S. Patent Application Publication No. 2010-0087529, where the Xaa residue at position 278 is Ala, Val, Ser, or Thr.

SEQ ID NO: 33 is the deduced amino acid sequence of a Thermotoga lettingae acetyl xylan esterase.

SEQ ID NO: 34 is the deduced amino acid sequence of a Thermotoga petrophila acetyl xylan esterase.

SEQ ID NO: 35 is the deduced amino acid sequence of a first acetyl xylan esterase from Thermotoga sp. RQ2 described herein as “RQ2(a)”.

SEQ ID NO: 36 is the deduced amino acid sequence of a second acetyl xylan esterase from Thermotoga sp. RQ2 described herein as “RQ2(b)”.

SEQ ID NO: 37 is the codon optimized nucleic acid sequence encoding a Thermoanearobacterium saccharolyticum cephalosporin C deacetylase.

SEQ ID NO: 38 is the deduced amino acid sequence of a Thermoanearobacterium saccharolyticum cephalosporin C deacetylase.

SEQ ID NO: 39 is the nucleic acid sequence encoding the acetyl xylan esterase from Lactococcus lactis (GENBANK® accession number EU255910).

SEQ ID NO: 40 is the amino acid sequence of the acetyl xylan esterase from Lactococcus lactis (GENBANK® accession number ABX75634.1).

SEQ ID NO: 41 is the nucleic acid sequence encoding the acetyl xylan esterase from Mesorhizobium loti (GENBANK® accession number NC_{—}002678.2).

SEQ ID NO: 42 is the amino acid sequence of the acetyl xylan esterase from Mesorhizobium loti (GENBANK® accession number BAB53179.1).

SEQ ID NO: 43 is the nucleic acid sequence encoding the acetyl xylan esterase from Geobacillus stearothermophilus (GENBANK® accession number AF038547.2).

SEQ ID NO: 44 is the amino acid sequence of the acetyl xylan esterase from Geobacillus stearothermophilus (GENBANK® accession number AAF70202.1).

SEQ ID NO: 45 is the nucleic acid sequence encoding a variant acetyl xylan esterase (a.k.a. variant “A3”) having the following substitutions relative to the wild-type Thermotoga maritime acetyl xylan esterase amino acid sequence: (F24I/S35T/Q179L/N275D/C277S/S308G/F317S).

SEQ ID NO: 46 is the amino acid sequence of the “A3” variant acetyl xylan esterase.

SEQ ID NO: 47 is the nucleic acid sequence encoding the N275D/C277S variant acetyl xylan esterase.

SEQ ID NO: 48 is the amino acid sequence of the N275D/C277S variant acetyl xylan esterase.

SEQ ID NO: 49 is the nucleic acid sequence encoding the C277S/F317S variant acetyl xylan esterase.

SEQ ID NO: 50 is the amino acid sequence of the C277S/F317S variant acetyl xylan esterase.

SEQ ID NO: 51 is the nucleic acid sequence encoding the S35T/C277S variant acetyl xylan esterase.

SEQ ID NO: 52 is the amino acid sequence of the S35T/C277S variant acetyl xylan esterase.

SEQ ID NO: 53 is the nucleic acid sequence encoding the Q179L/C277S variant acetyl xylan esterase.

SEQ ID NO: 54 is the amino acid sequence of the Q179L/C277S variant acetyl xylan esterase.

SEQ ID NO: 55 is the nucleic acid sequence encoding the variant acetyl xylan esterase 843H9 having the following substitutions relative to the wild-type Thermotoga maritime acetyl xylan esterase amino acid sequence: (L8R/L125Q/Q176L/V183D/F247I/C277S/P292L).

SEQ ID NO: 56 is the amino acid sequence of the 843H9 variant acetyl xylan esterase.

SEQ ID NO: 57 is the nucleic acid sequence encoding the variant acetyl xylan esterase 843F12 having the following substitutions relative to the wild-type Thermotoga maritime acetyl xylan esterase amino acid sequence: K77E/A266E/C277S.

SEQ ID NO: 58 is the amino acid sequence of the 843F12 variant acetyl xylan esterase.

SEQ ID NO: 59 is the nucleic acid sequence encoding the variant acetyl xylan esterase 843C12 having the following substitutions relative to the wild-type Thermotoga maritime acetyl xylan esterase amino acid sequence: F27Y/I149V/A266V/C277S/I295T/N302S.

SEQ ID NO: 60 is the amino acid sequence of the 843C12 variant acetyl xylan esterase.

SEQ ID NO: 61 is the nucleic acid sequence encoding the variant acetyl xylan esterase 842H3 having the following substitutions relative to the wild-type Thermotoga maritime acetyl xylan esterase amino acid sequence: L195Q/C277S.

SEQ ID NO: 62 is the amino acid sequence of the 842H3 variant acetyl xylan esterase.

SEQ ID NO: 63 is the nucleic acid sequence encoding the variant acetyl xylan esterase 841A7 having the following substitutions relative to the wild-type Thermotoga maritime acetyl xylan esterase amino acid sequence: Y110F/C277S.

SEQ ID NO: 64 is the amino acid sequence of the 841A7 variant acetyl xylan esterase.

SEQ ID NOs: 65-221, 271, 290, and 291 are a non-limiting list of amino acid sequences of peptides having affinity for hair.

SEQ ID NO: 217-269 are the amino acid sequences of peptides having affinity for skin.

SEQ ID NOs: 270-271 are the amino acid sequences of peptides having affinity for nail.

SEQ ID NOs: 272-285 are the amino acid sequences peptide linkers/spacers.

SEQ ID NO: 286 is the nucleic acid sequence encoding fusion peptide C277S-HC263.

SEQ ID NO: 287 is the nucleic acid sequence encoding the fusion construct C277S-HC1010.

SEQ ID NO: 288 is the amino acid sequence of fusion peptide C277S-HC263.

SEQ ID NO: 289 is the amino acid sequence of fusion peptide C277S-HC1010.

SEQ ID NO: 290 is the amino acid of hair-binding domain HC263.

SEQ ID NO: 291 is the amino acid sequence of hair-binding domain HC1010.

SEQ ID NO: 292 if the nucleic acid sequence of expression plasmid pLD001.

SEQ ID NO: 293 is the amino acid sequence of T. maritime variant C277S.

SEQ ID NO: 294 is the amino acid sequence of fusion peptide C277S-HC263 further comprising a D128G substitution (“CPAH-HC263”).

SEQ ID NO: 295 is the amino acid sequence of fusion peptide C277S-HC1010 further comprising a D128G substitution (“CPAH-HC1010”).

SEQ ID NO: 296 is the nucleic acid sequence encoding the variant acetyl xylan esterase 006A10 (U.S. Provisional Patent Appl. No. 61/425,561; hereby incorporated by reference) having the following substitutions relative to the wild-type Thermotoga maritime acetyl xylan esterase amino acid sequence: (F268S/C277T).

SEQ ID NO: 297 is the amino acid sequence of the 006A10 variant acetyl xylan esterase.

SEQ ID NO: 298 is the nucleic acid sequence encoding the variant acetyl xylan esterase 006E10 (U.S. Provisional Patent Appl. No. 61/425,561) having the following substitutions relative to the wild-type Thermotoga maritime acetyl xylan esterase amino acid sequence: (R218C/C277T/F317L).

SEQ ID NO: 299 is the amino acid sequence of the 006E10 variant acetyl xylan esterase.

SEQ ID NO: 300 is the nucleic acid sequence encoding the variant acetyl xylan esterase 006E12 (U.S. Provisional Patent Appl. No. 61/425,561) having the following substitutions relative to the wild-type Thermotoga maritime acetyl xylan esterase amino acid sequence: (H227L/T233A/C277T/A290V).

SEQ ID NO: 301 is the amino acid sequence of the 006E12 variant acetyl xylan esterase.

SEQ ID NO: 302 is the nucleic acid sequence encoding the variant acetyl xylan esterase 006G11(U.S. Provisional Patent Appl. No. 61/425,561) having the following substitutions relative to the wild-type Thermotoga maritime acetyl xylan esterase amino acid sequence: (D254G/C277T).

SEQ ID NO: 303 is the amino acid sequence of the 006G11 variant acetyl xylan esterase.

SEQ ID NO: 304 is the nucleic acid sequence encoding the variant acetyl xylan esterase 006F12 (U.S. Provisional Patent Appl. No. 61/425,561) having the following substitutions relative to the wild-type Thermotoga maritime acetyl xylan esterase amino acid sequence: (R261S/1264F/C277T).

SEQ ID NO: 305 is the amino acid sequence of the 006F12 variant acetyl xylan esterase.

SEQ ID NO: 306 is the nucleic acid sequence encoding the variant acetyl xylan esterase 006B12 (U.S. Provisional Patent Appl. No. 61/425,561) having the following substitutions relative to the wild-type Thermotoga maritime acetyl xylan esterase amino acid sequence: (W28C/F104S/C277T).

SEQ ID NO: 307 is the amino acid sequence of the 006B12 variant acetyl xylan esterase.

SEQ ID NO: 308 is the nucleic acid sequence encoding the variant acetyl xylan esterase 874B4 (U.S. Provisional Patent Appl. No. 61/425,561; hereby incorporated by reference) having the following substitutions relative to the wild-type Thermotoga maritime acetyl xylan esterase amino acid sequence: (A266P/C277S).

SEQ ID NO: 309 is the amino acid sequence of the 873B4 variant acetyl xylan esterase.

SEQ ID NO: 310 is the nucleic acid sequence encoding the variant acetyl xylan esterase 006D10 (U.S. Provisional Patent Appl. No. 61/425,561; hereby incorporated by reference) having the following substitutions relative to the wild-type Thermotoga maritime acetyl xylan esterase amino acid sequence: (W28C/L32P/D151E/C277T).

SEQ ID NO: 311 is the amino acid sequence of the 006D10 variant acetyl xylan esterase.

SEQ ID NO: 312 is the amino acid sequence of hair-binding domain “HC263KtoR”, a variant of hair binding domain “HC263” (SEQ ID NO: 290) in which 10 lysine residues have been replaced by 10 arginine residues.

SEQ ID NO: 313 is the amino acid sequence of the charged peptide (GK)₅-H6.

SEQ ID NO: 314 is the amino acid sequence of the S54V variant of the aryl esterase from Mycobacterium smegmatis.

SEQ ID NO: 315 is the amino acid sequence of the L29P variant of the hydrolase from Pseudomonas fluorescens.

SEQ ID NO: 316 is the nucleotide sequence of the synthetic gene encoding the acetyl xylan esterase from Bacillus pumilus fused at its C-terminus to the hair binding domain HC263 via a flexible linker.

SEQ ID NO: 317 is the amino acid sequence of the acetyl xylan esterase from Bacillus pumilus fused at its C-terminus to the hair binding domain HC263 via a flexible linker.

SEQ ID NO: 318 is the nucleotide sequence of the synthetic gene encoding the acetyl xylan esterase from Lactococcus lactis fused at its C-terminus to the hair binding domain HC263 via a flexible linker.

SEQ ID NO: 319 is the amino acid sequence of the acetyl xylan esterase from Lactococcus lactis fused at its C-terminus to the hair binding domain HC263 via a flexible linker.

SEQ ID NO: 320 is the nucleotide sequence of the synthetic gene encoding the acetyl xylan esterase from Mesorhizobium loti fused at its C-terminus to the hair binding domain HC263 via a flexible linker.

SEQ ID NO: 321 is the amino acid sequence of the acetyl xylan esterase from Mesorhizobium loti fused at its C-terminus to the hair binding domain HC263 via a flexible linker.

SEQ ID NO: 322 is the nucleotide sequence of the synthetic gene encoding the S54V variant of the aryl esterase from Mycobacterium smegmatis fused at its C-terminus to the hair binding domain HC263 via a flexible linker.

SEQ ID NO: 323 is the amino acid sequence of the S54V variant of the aryl esterase from Mycobacterium smegmatis fused at its C-terminus to the hair binding domain HC263 via a flexible linker.

SEQ ID NO: 324 is the nucleotide sequence of the synthetic gene encoding the S54V variant of the aryl esterase from Mycobacterium smegmatis fused at its C-terminus to the hair binding domain HC263KtoR via a flexible linker.

SEQ ID NO: 325 is the amino acid sequence of the S54V variant of the aryl esterase from Mycobacterium smegmatis fused at its C-terminus to the hair binding domain HC263KtoR via a flexible linker.

SEQ ID NO: 326 is the nucleotide sequence of the synthetic gene encoding the S54V variant of the aryl esterase from Mycobacterium smegmatis fused at its C-terminus to the hair binding domain HC1010 (SEQ ID NO: 291) via a flexible linker.

SEQ ID NO: 327 is the amino acid sequence of the S54V variant of the aryl esterase from Mycobacterium smegmatis fused at its C-terminus to the hair binding domain HC1010 via a flexible linker.

SEQ ID NO: 328 is the nucleotide sequence of the synthetic gene encoding the S54V variant of the aryl esterase from Mycobacterium smegmatis fused at its C-terminus to the charged peptide (GK)₅-His6 via a flexible linker.

SEQ ID NO: 329 is the amino acid sequence of the S54V variant of the aryl esterase from Mycobacterium smegmatis fused at its C-terminus to the charged peptide (GK)₅-His6 via a flexible linker.

SEQ ID NO: 330 is the nucleotide sequence of the synthetic gene encoding the L29P variant of the hydrolase from Pseudomonas fluorescens fused at its C-terminus to the hair binding domain HC263 via a flexible linker.

SEQ ID NO: 331 is the amino acid sequence of the L29P variant of the hydrolase from Pseudomonas fluorescens fused at its C-terminus to the hair binding domain HC263 via a flexible linker.

SEQ ID NO: 332 is the nucleotide sequence of the synthetic gene encoding the L29P variant of the hydrolase from Pseudomonas fluorescens fused at its C-terminus to the hair binding domain HC263KtoR via a flexible linker.

SEQ ID NO: 333 is the amino acid sequence of the L29P variant of the hydrolase from Pseudomonas fluorescens fused at its C-terminus to the hair binding domain HC263FtoR via a flexible linker.

SEQ ID NO: 334 is the nucleotide sequence of the synthetic gene encoding the L29P variant of the hydrolase from Pseudomonas fluorescens fused at its C-terminus to the hair binding domain HC1010 (SEQ ID NO: 291) via a flexible linker.

SEQ ID NO: 335 is the amino acid sequence of the L29P variant of the hydrolase from Pseudomonas fluorescens fused at its C-terminus to the hair binding domain HC1010 via a flexible linker.

SEQ ID NO: 336 is the nucleotide sequence of the synthetic gene encoding the L29P variant of the hydrolase from Pseudomonas fluorescens fused at its C-terminus to the charged peptide (GK)₅-His6 via a flexible linker.

SEQ ID NO: 337 is the amino acid sequence of the L29P variant of the hydrolase from Pseudomonas fluorescens fused at its C-terminus to the charged peptide (GK)₅-His6 via a flexible linker.

SEQ ID NO: 338 is the amino acid sequence of the wild type Mycobacterium smegmatis aryl esterase.

SEQ ID NO: 339 is the amino acid sequence of the wild type Pseudomonas fluorescens esterase.

### DETAILED DESCRIPTION OF THE INVENTION

In this disclosure, a number of terms and abbreviations are used. The following definitions apply unless specifically stated otherwise.

As used herein, the articles “a”, “an”, and “the” preceding an element or component of the invention are intended to be nonrestrictive regarding the number of instances (i.e., occurrences) of the element or component. Therefore “a”, “an”, and “the” should be read to include one or at least one, and the singular word form of the element or component also includes the plural unless the number is obviously meant to be singular.

As used herein, the term “comprising” means the presence of the stated features, integers, steps, or components as referred to in the claims, but that it does not preclude the presence or addition of one or more other features, integers, steps, components or groups thereof. The term “comprising” is intended to include embodiments encompassed by the terms “consisting essentially of” and “consisting of”. Similarly, the term “consisting essentially of” is intended to include embodiments encompassed by the term “consisting of”.

As used herein, the term “about” modifying the quantity of an ingredient or reactant employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities.

Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like.

As used herein, “contacting” refers to placing a composition in contact with the target body surface for a period of time sufficient to achieve the desired result (target surface binding, peracid based effects, etc). In one embodiment, “contacting” may refer to placing a composition comprising (or capable of producing) an efficacious concentration of peracid in contact with a target body surface for a period of time sufficient to achieve the desired result. In another embodiment, “contacting” may also refer to the placing at least one component of a personal care composition, such as one or more of the reaction components used to enzymatic perhydrolysis, in contact with a target body surface. Contacting includes spraying, treating, immersing, flushing, pouring on or in, mixing, combining, painting, coating, applying, affixing to and otherwise communicating a peracid solution or a composition comprising an efficacious concentration of peracid, a solution or composition that forms an efficacious concentration of peracid or a component of the composition that forms an efficacious concentration of peracid with the body surface.

As used herein, the terms “substrate”, “suitable substrate”, and “carboxylic acid ester substrate” interchangeably refer specifically to:
(a) one or more esters having the structure

[X]ₘR₅
wherein
X is an ester group of the formula R₆C(O)O;
R₆ is a C1 to C7 linear, branched or cyclic hydrocarbyl moiety, optionally substituted with a hydroxyl group or C1 to C4 alkoxy group, wherein R₆ optionally comprises one or more ether linkages where R₆ is C2 to C7;
R₅ is a C1 to C6 linear, branched, or cyclic hydrocarbyl moiety or a cyclic five-membered heteroaromatic or six-membered cyclic aromatic or heteroaromatic moiety optionally substituted with a hydroxyl group; wherein each carbon atom in R₅ individually comprises no more than one hydroxyl group or no more than one ester or carboxylic acid group, and wherein R₅ optionally comprises one or more ether linkages;
m is 1 to the number of carbon atoms in R₅,
said one or more esters having solubility in water of at least 5 ppm at 25° C.; or
(b) one or more glycerides having the structure

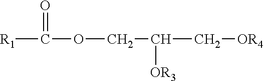

wherein R₁ is a C1 to C7 straight chain or branched chain alkyl optionally substituted with an hydroxyl or a C1 to C4 alkoxy group and R₃ and R₄ are individually H or R₁C(O); or
(c) one or more esters of the formula

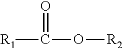

wherein R₁ is a C1 to C7 straight chain or branched chain alkyl optionally substituted with an hydroxyl or a C1 to C4 alkoxy group and R₂ is a C1 to C10 straight chain or branched chain alkyl, alkenyl, alkynyl, aryl, alkylaryl, alkylheteroaryl, heteroaryl, (CH₂CH₂O)ₙ, or (CH₂CH(CH₃)—O)ₙH and n is 1 to 10; or
(d) one or more acetylated monosaccharides, acetylated disaccharides, or acetylated polysaccharides; or
(e) any combination of (a) through (d).

As used herein, the term “peracid” is synonymous with peroxyacid, peroxycarboxylic acid, peroxy acid, percarboxylic acid and peroxoic acid.

As used herein, the term “peracetic acid” is abbreviated as “PAA” and is synonymous with peroxyacetic acid, ethaneperoxoic acid and all other synonyms of CAS Registry Number 79-21-0.

As used herein, the term “monoacetin” is synonymous with glycerol monoacetate, glycerin monoacetate, and glyceryl monoacetate.

As used herein, the term “diacetin” is synonymous with glycerol diacetate; glycerin diacetate, glyceryl diacetate, and all other synonyms of CAS Registry Number 25395-31-7.

As used herein, the term “triacetin” is synonymous with glycerin triacetate; glycerol triacetate; glyceryl triacetate, 1,2,3-triacetoxypropane; 1,2,3-propanetriol triacetate and all other synonyms of CAS Registry Number 102-76-1.

As used herein, the term “monobutyrin” is synonymous with glycerol monobutyrate, glycerin monobutyrate, and glyceryl monobutyrate.

As used herein, the term “dibutyrin” is synonymous with glycerol dibutyrate and glyceryl dibutyrate.

As used herein, the term “tributyrin” is synonymous with glycerol tributyrate, 1,2,3-tributyrylglycerol, and all other synonyms of CAS Registry Number 60-01-5.

As used herein, the term “monopropionin” is synonymous with glycerol monopropionate, glycerin monopropionate, and glyceryl monopropionate.

As used herein, the term “dipropionin” is synonymous with glycerol dipropionate and glyceryl dipropionate.

As used herein, the term “tripropionin” is synonymous with glyceryl tripropionate, glycerol tripropionate, 1,2,3-tripropionylglycerol, and all other synonyms of CAS Registry Number 139-45-7.

As used herein, the terms “acetylated sugar” and “acetylated saccharide” refer to mono-, di- and polysaccharides comprising at least one acetyl group. Examples include, but are not limited to glucose pentaacetate; xylose tetraacetate; acetylated xylan; acetylated xylan fragments; β-D-ribofuranose-1,2,3,5-tetraacetate; tri-O-acetyl-D-galactal; and tri-O-acetyl-glucal.

As used herein, the terms “hydrocarbyl”, “hydrocarbyl group”, and “hydrocarbyl moiety” is meant a straight chain, branched or cyclic arrangement of carbon atoms connected by single, double, or triple carbon to carbon bonds and/or by ether linkages, and substituted accordingly with hydrogen atoms. Such hydrocarbyl groups may be aliphatic and/or aromatic. Examples of hydrocarbyl groups include methyl, ethyl, propyl, isopropyl, butyl, isobutyl, t-butyl, cyclopropyl, cyclobutyl, pentyl, cyclopentyl, methylcyclopentyl, hexyl, cyclohexyl, benzyl, and phenyl. In a preferred embodiment, the hydrocarbyl moiety is a straight chain, branched or cyclic arrangement of carbon atoms connected by single carbon to carbon bonds and/or by ether linkages, and substituted accordingly with hydrogen atoms.

As used herein, the terms “monoesters” and “diesters” of 1,2-ethanediol; 1,2-propanediol; 1,3-propanediol; 1,2-butanediol; 1,3-butanediol; 2,3-butanediol; 1,4-butanediol; 1,2-pentanediol; 2,5-pentanediol; 1,5-pentanediol; 1,6-pentanediol; 1,2-hexanediol; 2,5-hexanediol; 1,6-hexanediol; and mixtures thereof, refer to said compounds comprising at least one ester group of the formula RC(O)O, wherein R is a C1 to C7 linear hydrocarbyl moiety. In one embodiment, the carboxylic acid ester substrate is selected from the group consisting of propylene glycol diacetate (PGDA), ethylene glycol diacetate (EDGA), and mixtures thereof.

As used herein, the term “propylene glycol diacetate” is synonymous with 1,2-diacetoxypropane, propylene diacetate, 1,2-propanediol diacetate, and all other synonyms of CAS Registry Number 623-84-7.

As used herein, the term “ethylene glycol diacetate” is synonymous with 1,2-diacetoxyethane, ethylene diacetate, glycol diacetate, and all other synonyms of CAS Registry Number 111-55-7.

As used herein, the terms “suitable enzymatic reaction mixture”, “components suitable for in situ generation of a peracid”, “suitable reaction components”, “suitable aqueous reaction mixture”, “reaction mixture”, and “peracid-generating components” refer to the materials and water in which the reactants and the perhydrolytic enzyme catalyst come into contact. In one embodiment, the peracid-generating components will include at least one perhydrolase, preferably in the form of a fusion protein comprising a binding domain having affinity for a body surface such as hair, at least one suitable carboxylic acid ester substrate, a source of peroxygen, and water. In a preferred aspect, the perhydrolase is a CE-7 perhydrolase, preferable in the form of a fusion protein targeted to a body surface, such as hair.

As used herein, the term “perhydrolysis” is defined as the reaction of a selected substrate with peroxide to form a peracid. Typically, inorganic peroxide is reacted with the selected substrate in the presence of a catalyst to produce the peroxycarboxylic acid. As used herein, the term “chemical perhydrolysis” includes perhydrolysis reactions in which a substrate (a peroxycarboxylic acid precursor) is combined with a source of hydrogen peroxide wherein peroxycarboxylic acid is formed in the absence of an enzyme catalyst. As used herein, the term “enzymatic perhydrolysis” includes perhydrolysis reactions in which a carboxylic acid ester substrate (a peracid precursor) is combined with a source of hydrogen peroxide and water whereby the enzyme catalyst catalyzes the formation of peracid.

As used herein, the term “perhydrolase activity” refers to the catalyst activity per unit mass (for example, milligram) of protein, dry cell weight, or immobilized catalyst weight.

As used herein, “one unit of enzyme activity” or “one unit of activity” or “U” is defined as the amount of perhydrolase activity required for the production of 1 μmmol of peroxycarboxylic acid product per minute at a specified temperature.

As used herein, the terms “enzyme catalyst” and “perhydrolase catalyst” refer to a catalyst comprising an enzyme having perhydrolysis activity and may be in the form of a whole microbial cell, permeabilized microbial cell(s), one or more cell components of a microbial cell extract, partially purified enzyme, or purified enzyme. The enzyme catalyst may also be chemically modified (such as by pegylation or by reaction with cross-linking reagents). The perhydrolase catalyst may also be immobilized on a soluble or insoluble support using methods well-known to those skilled in the art; see for example, Immobilization of Enzymes and Cells; Gordon F. Bickerstaff, Editor; Humana Press, Totowa, N.J., USA; 1997.

As used herein, “acetyl xylan esterases” refers to an enzyme (E.G. 3.1.1.72; AXEs) that catalyzes the deacetylation of acetylated xylans and other acetylated saccharides.

As used herein, the terms “cephalosporin C deacetylase” and “cephalosporin C acetyl hydrolase” refer to an enzyme (E.G. 3.1.1.41) that catalyzes the deacetylation of cephalosporins such as cephalosporin C and 7-aminocephalosporanic acid (Mitsushima et al., (1995) Appl. Env. Microbiol. 61 (6):2224-2229).

As used herein, the term “Bacillus subtilis ATCC® 31954™” refers to a bacterial cell deposited to the American Type Culture Collection (ATCC) having international depository accession number ATCC® 31954™. An enzyme having significant perhydrolase activity from B. subtilis ATCC® 31954™ is provided as SEQ ID NO: 2 (see United States Patent Application Publication No. 2010-0041752). The amino acid sequence of the isolated enzyme has 100% amino acid identity to the cephalosporin C deacetylase provided by GENBANK® Accession No. BAA01729.1 (Mitsushima et al., supra).

As used herein, the term “Thermotoga maritime MSB8” refers to a bacterial cell reported to have acetyl xylan esterase activity (GENBANK® NP_{—}227893.1; see U.S. Patent Application Publication No. 2008-0176299). The amino acid sequence of the enzyme having perhydrolase activity from Thermotoga maritime MSB8 is provided as SEQ ID NO: 16.

The term “amino acid” refers to the basic chemical structural unit of a protein or polypeptide. The following abbreviations are used herein to identify specific amino acids:

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid or as defined herein | Xaa | X |

For example, it is well known in the art that alterations in a gene which result in the production of a chemically equivalent amino acid at a given site, but do not affect the functional properties of the encoded protein are common. For the purposes of the present invention substitutions are defined as exchanges within one of the following five groups:

1. Small aliphatic, nonpolar or slightly polar residues: Ala, Ser, Thr (Pro, Gly);

2. Polar, negatively charged residues and their amides: Asp, Asn, Glu, Gln;

3. Polar, positively charged residues: His, Arg, Lys;

4. Large aliphatic, nonpolar residues: Met, Leu, Ile, Val (Cys); and

5. Large aromatic residues: Phe, Tyr, and Trp.

Thus, a codon for the amino acid alanine, a hydrophobic amino acid, may be substituted by a codon encoding another less hydrophobic residue (such as glycine) or a more hydrophobic residue (such as valine, leucine, or isoleucine). Similarly, changes which result in substitution of one negatively charged residue for another (such as aspartic acid for glutamic acid) or one positively charged residue for another (such as lysine for arginine) can also be expected to produce a functionally equivalent product. In many cases, nucleotide changes which result in alteration of the N-terminal and C-terminal portions of the protein molecule would also not be expected to alter the activity of the protein. Each of the proposed modifications is well within the routine skill in the art, as is determination of retention of biological activity of the encoded products.

As used herein, the terms “signature motif” and “diagnostic motif” refer to conserved structures shared among a family of enzymes having a defined activity. The signature motif can be used to define and/or identify the family of structurally-related enzymes having similar enzymatic activity for a defined family of substrates. The signature motif can be a single contiguous amino acid sequence or a collection of discontiguous, conserved motifs that together form the signature motif. Typically, the conserved motifs) is represented by an amino acid sequence. In one embodiment, the perhydrolytic enzyme comprises a CE-7 carbohydrate esterase signature motif.

As used herein, the term “sequence analysis software” refers to any computer algorithm or software program that is useful for the analysis of nucleotide or amino acid sequences. “Sequence analysis software” may be commercially available or independently developed. Typical sequence analysis software will include, but is not limited to, the GCG suite of programs (Wisconsin Package Version 9.0, Genetics Computer Group (GCG), Madison, Wis.), BLASTP, BLASTN, BLASTX (Altschul et al., J. Mol. Biol. 215:403-410 (1990)), and DNASTAR (DNASTAR, Inc. 1228 S. Park St. Madison, Wis. 53715 USA), CLUSTALW (for example, version 1.83; Thompson et al., Nucleic Acids Research, 22(22):4673-4680 (1994)), and the FASTA program incorporating the Smith-Waterman algorithm (W. R. Pearson, Comput. Methods Genome Res., [Proc. Int. Symp.] (1994), Meeting Date 1992, 111-20. Editor(s): Suhai, Sandor. Publisher: Plenum, New York, N.Y.), Vector NTI (Informax, Bethesda, Md.) and Sequencher v. 4.05. Within the context of this application it will be understood that where sequence analysis software is used for analysis, that the results of the analysis will be based on the “default values” of the program referenced, unless otherwise specified. As used herein “default values” will mean any set of values or parameters set by the software manufacturer that originally load with the software when first initialized.

As used herein, the term “body surface” refers to any surface of the human body that may serve as the target for a benefit agent, such as a peracid benefit agent. Typical body surfaces include but are not limited to hair, skin, nails, teeth, and gums. The present methods and compositions are directed to hair care applications and products. As such, the body surface comprises hair. In one embodiment, the body surface is human hair.

As used herein, “personal care products” means products used in the cleaning, bleaching and/or disinfecting of hair, skin, scalp, and teeth, including, but not limited to shampoos, body lotions, shower gels, topical moisturizers, toothpaste, toothgels, mouthwashes, mouthrinses, anti-plaque rinses, and/or other topical cleansers. In some particularly preferred embodiments, these products are utilized on humans, while in other embodiments, these products find use with non-human animals (e.g., in veterinary applications). In a preferred embodiment, the term “personal care products” refers to hair care products or skin care products.

As used herein, the terms “peroxygen source” and “source of peroxygen” refer to compounds capable of providing hydrogen peroxide at a concentration of about 1 mM or more when present an aqueous solution including, but not limited to, hydrogen peroxide, hydrogen peroxide adducts (e.g., urea-hydrogen peroxide adduct (carbamide peroxide)), perborates, and percarbonates. The present hair care compositions and methods are specifically directed to the use of a solid peroxygen source that is stored in a solid form in a non-aqueous component comprising the carboxylic acid ester substrate while the enzyme catalyst having perhydrolytic activity is stored separately in an aqueous composition. The two compositions are combined to enzymatically generate the desired peracid. Typically, the amount of the solid source of the peroxygen used is specifically chosen such that the resulting working concentration of hydrogen peroxide that is released upon combining the reaction components is capable or providing an effective amount of hydrogen peroxide. In one embodiment, the resulting concentration of hydrogen peroxide provided upon combining the reaction components is initially at least 0.1 mM, 0.5 mM, 1 mM, 10 mM, 100 mM, 200 mM or 500 mM or more. The molar ratio of the hydrogen peroxide to enzyme substrate, e.g., triglyceride, (H₂O₂:substrate) in the aqueous reaction formulation may be from about 0.002 to 20, preferably about 0.1 to 10, and most preferably about 0.5 to 5.

As used herein, the term “excipient” refers to inactive substance used as a carrier for active ingredients in a formulation. The excipient may be used to stabilize the active ingredient in a formulation, such as the storage stability of the active ingredient. Excipients are also sometimes used to bulk up formulations that contain active ingredients. As described herein, the “active ingredient” may be an enzyme having perhydrolytic activity, a peracid produced by the perhydrolytic enzyme under suitable reaction conditions, or a combination thereof.

The present hair care product design comprises a first composition comprising (1) a solid form of peroxygen (e.g., percarbonate) stored in (2) a non-aqueous system (i.e., the carboxylic acid ester and optionally one or more organic cosolvents) and a second composition which is aqueous comprising the perhydrolytic enzyme catalyst and a buffer. In order to maintain stability of carboxylic acid ester in the presence of the solid source of peroxygen, the first composition is substantially free of water. The term “substantially free of water” will refer to a concentration of water in that does not adversely impact the storage stability of the carboxylic acid ester substrate when stored with the solid form of peroxygen. In a further embodiment, “substantially free of water” may mean less than 2000 ppm, preferably less than 1000 ppm, more preferably less than 500 ppm, and even more preferably less than 250 ppm of water in the component comprising the solid source of peroxygen and the carboxylic acid ester. In one embodiment, the perhydrolytic enzyme may be stored in an aqueous solution if the generation system is designed such that the enzyme is stable in the aqueous solution (for example, a solution that does not contain a significant concentration of a carboxylic acid ester substrate capable of being hydrolyzed by the enzyme during storage). In one embodiment, the perhydrolytic enzyme may be stored in an aqueous composition comprising one or more buffers capable of providing the desired pH for storage stability of the enzyme (e.g., sodium and/or potassium salts of bicarbonate, citrate, acetate, phosphate, pyrophosphate, methylphosphonate, succinate, malate, fumarate, tartrate, and maleate). In a preferred aspect, the buffer is capable of providing and maintaining a pH of 4 or more to the aqueous component comprising the enzyme.

### Enzymes Having Perhydrolytic Activity

Enzymes having perhydrolytic activity may include some enzymes classified as lipases, proteases, esterases, acyl transferases, aryl esterases, carbohydrate esterases, and combinations so long as the enzyme has perhydrolytic activity for one or more of the present substrates. Examples may include, but are not limited to perhydrolytic proteases (subtilisin Carlsberg variant; U.S. Pat. No. 7,510,859), perhydrolytic aryl esterases (Pseudomonas fluorescens; SEQ ID NO: 315 [L29P variant] and SEQ ID NO: 339 [wild type]; U.S. Pat. No. 7,384,787), a perhydrolytic aryl esterase from Mycobacterium smegmatis (SEQ ID NO: 314 [S54V variant] and SEQ ID NO: 338 [wild type]; U.S. Pat. No. 7,754,460; WO2005/056782; and EP1689859 B1), and perhydrolytic carbohydrate esterases. In one embodiment, the perhydrolytic enzyme comprises an amino acid sequence having at least 95% identity to the Mycobacterium smegmatis S54V aryl esterase provided as SEQ ID NO: 314. In a preferred aspect, the perhydrolytic carbohydrate esterase is a CE-7 carbohydrate esterase.

In one embodiment, suitable perhydrolases may include enzymes comprising an amino acid sequence having at least 30%, 33%, 40%, 50%, 60%, 70%, 80%, 85%, 90%, 91%, 92%, 93%, 94%, 95%, 96%, 97%, 98%, or 99% amino acid identity to any of the amino acid sequences encoding an enzyme having perhydrolytic activity as reported herein.

In another embodiment, the suitable perhydrolases may include enzymes comprising an amino acid sequence having at least 30%, 33%, 40%, 50%, 60%, 70%, 80%, 85%, 90%, 91%, 92%, 93%, 94%, 95%, 96%, 97%, 98%, or 99% amino acid identity to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 293, 297, 299, 301, 303, 305, 307, 309, 311, 314, 315, 338, and 339.

In one embodiment, the suitable perhydrolases may include enzymes comprising an amino acid sequence having at least 30%, 33%, 40%, 50%, 60%, 70%, 80%, 85%, 90%, 91%, 92%, 93%, 94%, 95%, 96%, 97%, 98%, or 99% amino acid identity to SEQ ID NO: 314, 315, 338, and 339.

In another embodiment, substantially similar perhydrolytic enzymes may include those encoded by polynucleotide sequences that hybridize under highly stringent hybridization conditions (0.1×SSC, 0.1% SDS, 65° C. and washed with 2×SSC, 0.1% SDS followed by a final wash of 0.1×SSC, 0.1% SDS, 65° C.) to the polynucleotide sequences encoding any of the present perhydrolytic enzymes.

In a preferred embodiment, the perhydrolases may be in the form of fusion proteins having at least one peptidic component having affinity for at least one body surface. In one embodiment, all alignments used to determine if a targeted perhydrolase (fusion protein) comprises a substantially similar sequence to any of the perhydrolases described herein are based on the amino acid sequence of the perhydrolytic enzyme without the peptidic component having the affinity for a body surface.

### CE-7 Perhydrolases

In a preferred embodiment, the present hair care compositions and methods comprise enzymes having perhydrolytic activity that are structurally classified as members of the carbohydrate family esterase family 7 (CE-7 family) of enzymes (see Coutinho, P. M., Henrissat, B. “Carbohydrate-active enzymes: an integrated database approach” in Recent Advances in Carbohydrate Bioengineering, H. J. Gilbert, G. Davies, B. Henrissat and B. Svensson eds., (1999) The Royal Society of Chemistry, Cambridge, pp. 3-12). The CE-7 family of enzymes has been demonstrated to be particularly effective for producing peroxycarboxylic acids from a variety of carboxylic acid ester substrates when combined with a source of peroxygen (WO2007/070609 and U.S. Patent Application Publication Nos. 2008-0176299, 2008-176783, 2009-0005590, 2010-0041752, and 2010-0087529, as well as U.S. patent application Ser. No. 12/571,702 and U.S. Provisional Patent Application No. 61/318,016 to DiCosimo et al.; each incorporated herein by reference).

Members of the CE-7 family include cephalosporin C deacetylases (CAHs; E.C. 3.1.1.41) and acetyl xylan esterases (AXEs; E.G. 3.1.1.72). Members of the CE-7 esterase family share a conserved signature motif (Vincent et al., J. Mol. Biol., 330:593-606 (2003)). Perhydrolases comprising the CE-7 signature motif (“CE-7 perhydrolases”) and/or a substantially similar structure are suitable for use in the compositions and methods described herein. Means to identify substantially similar biological molecules are well known in the art (e.g., sequence alignment protocols, nucleic acid hybridizations and/or the presence of a conserved signature motif). In one aspect, the perhydrolase includes an enzyme comprising the CE-7 signature motif and at least 20%, preferably at least 30%, more preferably at least 33%, more preferably at least 40%, more preferably at least 42%, more preferably at least 50%, more preferably at least 60%, more preferably at least 70%, more preferably at least 80%, more preferably at least 90%, and most preferably at least 90%, 91%, 92%, 93%, 94%, 95%, 96%, 97%, 98%, or 99% amino acid identity to one of the sequences provided herein.

As used herein, the phrase “enzyme is structurally classified as a CE-7 enzyme”, “CE-7 perhydrolase” or “structurally classified as a carbohydrate esterase family 7 enzyme” will be used to refer to enzymes having perhydrolysis activity which are structurally classified as a CE-7 carbohydrate esterase. This family of enzymes can be defined by the presence of a signature motif (Vincent et al., supra). The signature motif for CE-7 esterases comprises three conserved motifs (residue position numbering relative to reference sequence SEQ ID NO: 2; the CE-7 perhydrolase from B. subtilis ATCC® 31954™):

a) Arg118-Gly119-Gln120;

b) Gly179-Xaa180-Ser181-Gln182-Gly183; and

c) His298-Glu299.

Typically, the Xaa at amino acid residue position 180 is glycine, alanine, proline, tryptophan, or threonine. Two of the three amino acid residues belonging to the catalytic triad are in bold. In one embodiment, the Xaa at amino acid residue position 180 is selected from the group consisting of glycine, alanine, proline, tryptophan, and threonine.

Further analysis of the conserved motifs within the CE-7 carbohydrate esterase family indicates the presence of an additional conserved motif (LXD at amino acid positions 267-269 of SEQ ID NO: 2) that may be used to further define a perhydrolase belonging to the CE-7 carbohydrate esterase family. In a further embodiment, the signature motif defined above may include an additional (fourth) conserved motif defined as:

Leu267-Xaa268-Asp269.

The Xaa at amino acid residue position 268 is typically isoleucine, valine, or methionine. The fourth motif includes the aspartic acid residue (bold) belonging to the catalytic triad (Ser181-Asp269-His298).

The CE-7 perhydrolases may be in the form of fusion proteins having at least one peptidic component having affinity for at least one body surface. In one embodiment, all alignments used to determine if a targeted perhydrolase (fusion protein) comprises the CE-7 signature motif will be based on the amino acid sequence of the perhydrolytic enzyme without the peptidic component having the affinity for a body surface.

A number of well-known global alignment algorithms (i.e., sequence analysis software) may be used to align two or more amino acid sequences representing enzymes having perhydrolase activity to determine if the enzyme is comprised of the present signature motif. The aligned sequence(s) are compared to the reference sequence (SEQ ID NO: 2) to determine the existence of the signature motif. In one embodiment, a CLUSTAL alignment (such as CLUSTALW) using a reference amino acid sequence (as used herein the perhydrolase sequence (SEQ ID NO: 2) from the Bacillus subtilis ATCC® 31954™) is used to identify perhydrolases belonging to the CE-7 esterase family. The relative numbering of the conserved amino acid residues is based on the residue numbering of the reference amino acid sequence to account for small insertions or deletions (for example, typically five amino acids of less) within the aligned sequence.

Examples of other suitable algorithms that may be used to identify sequences comprising the present signature motif (when compared to the reference sequence) include, but are not limited to, Needleman and Wunsch (J. Mol. Biol. 48, 443-453 (1970); a global alignment tool) and Smith-Waterman (J. Mol. Biol. 147:195-197 (1981); a local alignment tool). In one embodiment, a Smith-Waterman alignment is implemented using default parameters. An example of suitable default parameters include the use of a BLOSUM62 scoring matrix with GAP open penalty=10 and a GAP extension penalty=0.5.

A comparison of the overall percent identity among perhydrolases indicates that enzymes having as little as approximately 30% amino acid identity to SEQ ID NO: 2 (while retaining the signature motif) exhibit significant perhydrolase activity and are structurally classified as CE-7 carbohydrate esterases. In one embodiment, suitable perhydrolases include enzymes comprising the CE-7 signature motif and at least 20%, preferably at least 30%, 33%, 40%, 50%, 60%, 70%, 80%, 85%, 90%, 91%, 92%, 93%, 94%, 95%, 96%, 97%, 98%, or 99% amino acid identity to SEQ ID NO: 2.

Examples of suitable CE-7 carbohydrate esterases having perhydrolytic activity include, but are not limited to, enzymes having an amino acid sequence such as SEQ ID NOs: 2, 4, 6, 8, 10, 12, 14, 16, 18, 20, 22, 24, 26, 27, 28, 29, 30, 31, 32, 33, 34, 35, 36, 38, 40, 42, 44, 46, 48, 50, 52, 54, 56, 58, 60, 62, 64, 293, 297, 299, 301, 303, 305, 307, 309, and 311. In one embodiment, the enzyme comprises an amino acid sequence selected from the group consisting of 14, 16, 27, 28, 29, 30, 31, 32, 33, 34, 35, 36, 46, 48, 50, 52, 54, 56, 58, 60, 62, and 64. In a further preferred embodiment, the CE-7 carbohydrate esterase is derived from the Thermotoga maritime CE-7 carbohydrate esterase (SEQ ID NO: 16).

As used herein, the term “CE-7 variant”, “variant perhydrolase” or “variant” will refer to CE-7 perhydrolases having a genetic modification that results in at least one amino acid addition, deletion, and/or substitution when compared to the corresponding enzyme (typically the wild type enzyme) from which the variant was derived; so long as the CE-7 signature motif and the associated perhydrolytic activity are maintained. CE-7 variant perhydrolases may also be used in the present compositions and methods. Examples of CE-7 variants are provided as SEQ ID NOs: 27, 28, 29, 30, 31, 32, 48, 50, 52, 54, 56, 58, 60, 62, 64, 293, 297, 299, 301, 303, 305, 307, 309, and 311. In one embodiment, the variants may include SEQ ID NOs: 27, 28, 50, 52, 54, 56, 58, 60, 62, and 64.

The skilled artisan recognizes that substantially similar CE-7 perhydrolase sequences (retaining the signature motifs) may also be used in the present compositions and methods. In one embodiment, substantially similar sequences are defined by their ability to hybridize, under highly stringent conditions with the nucleic acid molecules associated with sequences exemplified herein. In another embodiment, sequence alignment algorithms may be used to define substantially similar enzymes based on the percent identity to the DNA or amino acid sequences provided herein.

As used herein, a nucleic acid molecule is “hybridizable” to another nucleic acid molecule, such as a cDNA, genomic DNA, or RNA, when a single strand of the first molecule can anneal to the other molecule under appropriate conditions of temperature and solution ionic strength. Hybridization and washing conditions are well known and exemplified in Sambrook, J. and Russell, D., T. Molecular Cloning: A Laboratory Manual, Third Edition, Cold Spring Harbor Laboratory Press, Cold Spring Harbor (2001). The conditions of temperature and ionic strength determine the “stringency” of the hybridization. Stringency conditions can be adjusted to screen for moderately similar molecules, such as homologous sequences from distantly related organisms, to highly similar molecules, such as genes that duplicate functional enzymes from closely related organisms. Post-hybridization washes typically determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of conditions uses higher temperatures in which the washes are identical to those above except for the temperature of the final two 30 min washes in 0.2×SSC, 0.5% SDS was increased to 60° C. Another preferred set of highly stringent hybridization conditions is 0.1×SSC, 0.1% SDS, 65° C. and washed with 2×SSC, 0.1% SDS followed by a final wash of 0.1×SSC, 0.1% SDS, 65° C.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (Sambrook and Russell, supra). For hybridizations with shorter nucleic acids, i.e., oligonucleotides, the position of mismatches becomes more important, and the length of the oligonucleotide determines its specificity (Sambrook and Russell, supra). In one aspect, the length for a hybridizable nucleic acid is at least about 10 nucleotides. Preferably, a minimum length for a hybridizable nucleic acid is at least about 15 nucleotides in length, more preferably at least about 20 nucleotides in length, even more preferably at least 30 nucleotides in length, even more preferably at least 300 nucleotides in length, and most preferably at least 800 nucleotides in length. Furthermore, the skilled artisan will recognize that the temperature and wash solution salt concentration may be adjusted as necessary according to factors such as length of the probe.

As used herein, the term “percent identity” is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Methods to determine identity and similarity are codified in publicly available computer programs. Sequence alignments and percent identity calculations may be performed using the Megalign program of the LASERGENE bioinformatics computing suite (DNASTAR Inc., Madison, Wis.), the AlignX program of Vector NTI v. 7.0 (Informax, Inc., Bethesda, Md.), or the EMBOSS Open Software Suite (EMBL-EBI; Rice et al., Trends in Genetics 16, (6):276-277 (2000)). Multiple alignment of the sequences can be performed using the CLUSTAL method (such as CLUSTALW; for example version 1.83) of alignment (Higgins and Sharp, CABIOS, 5:151-153 (1989); Higgins et al., Nucleic Acids Res. 22:4673-4680 (1994); and Chema et al., Nucleic Acids Res 31 (13):3497-500 (2003)), available from the European Molecular Biology Laboratory via the European Bioinformatics Institute) with the default parameters. Suitable parameters for CLUSTALW protein alignments include GAP Existence penalty=15, GAP extension=0.2, matrix=Gonnet (e.g., Gonnet250), protein ENDGAP=−1, protein GAPDIST=4, and KTUPLE=1. In one embodiment, a fast or slow alignment is used with the default settings where a slow alignment is preferred. Alternatively, the parameters using the CLUSTALW method (e.g., version 1.83) may be modified to also use KTUPLE=1, GAP PENALTY=10, GAP extension=1, matrix=BLOSUM (e.g., BLOSUM64), WINDOW=5, and TOP DIAGONALS SAVED=5.

In one aspect, suitable isolated nucleic acid molecules encode a polypeptide having an amino acid sequence that is at least about 20%, preferably at least 30%, 33%, 40%, 50%, 60%, 70%, 80%, 85%, 90%, 91%, 92%, 93%, 94%, 95%, 96%, 97%, 98%, or 99% identical to the amino acid sequences reported herein. In another aspect, suitable isolated nucleic acid molecules encode a polypeptide having an amino acid sequence that is at least about 20%, preferably at least 30%, 33%, 40%, 50%, 60%, 70%, 80%, 85%, 90%, 91%, 92%, 93%, 94%, 95%, 96%, 97%, 98%, or 99% identical to the amino acid sequences reported herein; with the proviso that the polypeptide retains the CE-7 signature motif. Suitable nucleic acid molecules not only have the above homologies, but also typically encode a polypeptide having about 210 to 340 amino acids in length, about 300 to about 340 amino acids, preferably about 310 to about 330 amino acids, and most preferably about 318 to about 325 amino acids in length wherein each polypeptide is characterized as having perhydrolytic activity.

### Targeted Perhydrolases

As used herein, the term “targeted perhydrolase” and “targeted enzyme having perhydrolytic activity” will refer to a fusion proteins comprising at least one perhydrolytic enzyme (wild type or variant thereof) fused/coupled to at least one peptidic component having affinity for a target surface, preferably a targeted body surface. The perhydrolytic enzyme within the targeted perhydrolase may be any perhydrolytic enzyme and may include lipases, proteases, esterases, acyl transferases, aryl esterases, carbohydrate esterases, and combinations so long as the enzyme has perhydrolytic activity for one or more of the present substrates. Examples may include, but are not limited to perhydrolytic proteases (subtilisin variant; U.S. Pat. No. 7,510,859), perhydrolytic esterase (Pseudomonas fluorescens; U.S. Pat. No. 7,384,787; SEQ ID NO: 315 [L29P variant] and SEQ ID NO: 339 [wild type]), a perhydrolytic aryl esterase (Mycobacterium smegmatis; U.S. Pat. No. 7,754,460; WO2005/056782; and EP1689859 B1; SEQ ID NOs: 314 [S54V variant] and 338 [wild type]).

As used herein the terms “at least one binding domain having affinity for hair”, “peptidic component having affinity for a body surface”, “peptidic component having affinity for hair”, and “HSBD” will refer to a peptidic component of a fusion protein that is not part of the perhydrolytic enzyme comprising at least one polymer of two or more amino acids joined by a peptide bond; wherein the component has affinity for hair, preferably human hair.

In one embodiment, the peptidic component having affinity for a body surface may be an antibody, an F_{ab} antibody fragment, a single chain variable fragment (scFv) antibody, a Camelidae antibody (Muyldermans, S., Rev. Mol. Biotechnol. (2001) 74:277-302), a non-antibody scaffold display protein (Hosse et al., Prot. Sci. (2006) 15(1): 14-27 and Binz, H. et al. (2005) Nature Biotechnology 23, 1257-1268 for a review of various scaffold-assisted approaches) or a single chain polypeptide lacking an immunoglobulin fold. In another aspect, the peptidic component having affinity for a body surface is a single chain peptide lacking an immunoglobulin fold (i.e., a body surface-binding peptide or a body surface-binding domain comprising at least one body surface-binding peptide having affinity for hair). In a preferred embodiment, the peptidic component is a single chain peptide lacking an immunoglobulin fold comprising one or more body surface-binding peptides having affinity for hair.

The peptidic component having affinity for hair may be separated from the perhydrolytic enzyme by an optional peptide linker. Certain peptide linkers/spacers are from 1 to 100 or 1 to 50 amino acids in length. In some embodiments, the peptide spacers are about 1 to about 25, 3 to about 40, or 3 to about 30 amino acids in length. In other embodiments are spacers that are about 5 to about 20 amino acids in length.

In one embodiment, the peptidic component having affinity for hair may include one or more hair-binding peptide, each optionally and independently separated by a peptide spacer of 1 to 100 amino acids in length. Examples of hair-binding peptides and/or hair-binding domains comprising a hair-binding peptide may include, but are not limited to SEQ ID NOs: 65-221, 271, 290, 291, 312, and 313. Examples of peptide linkers/spacer may include, but are not limited to SEQ ID NOs: 272 through 285.

Peptides previously identified as having affinity for one body surface may have affinity for the hair as well. As such, the fusion peptide may comprise at least one previously reported to have affinity for another body surface, such as skin (SEQ ID NOs: 217-269) or nail (SEQ ID NOs: 270-271). In another embodiment, the fusion peptide may include any body surface-binding peptide designed to have electrostatic attraction to the target body surface (e.g., a body surface-binding peptide engineered to electrostatically bind to the target body surface).

In one embodiment, examples of targeted perhydrolytic enzymes may include one or more of SEQ ID NOs: 288, 289, 294, 295, 317, 319, 321, 323, 325, 327, 329, 331, 333, 335, and 337. In a preferred embodiment, the examples of targeted perhydrolytic enzymes may include one or more of SEQ ID NOs: 288, 289, 294, 295, 317, 319, 321, 323, 325, 327, and 329.

### Targeted CE-7 Perhydrolases

In a preferred embodiment, the “targeted perhydrolase” is a targeted CE-7 carbohydrate esterase having perhydrolytic activity. As used herein, the terms “targeted CE-7 perhydrolase” and “targeted CE-7 carbohydrate esterase” will refer to fusion proteins comprising at least one CE-7 perhydrolase (wild type or variant perhydrolase) fused/coupled to at least one peptidic component having affinity for a targeted surface, preferably hair. The peptidic component having affinity for a body surface may be any of those describe above. In a preferred aspect, the peptidic component in a targeted CE-7 perhydrolase is a single chain peptide lacking an immunoglobulin fold (i.e., a body surface-binding peptide or a body surface-binding domain comprising at least one body surface-binding peptide having affinity for hair). In a preferred embodiment, the peptidic component is a single chain peptide lacking an immunoglobulin fold comprising one or more body surface-binding peptides having affinity for hair.

The peptidic component having affinity for hair/hair surface may be separated from the CE-7 perhydrolase by an optional peptide linker. Certain peptide linkers/spacers are from 1 to 100 or 1 to 50 amino acids in length. In some embodiments, the peptide spacers are about 1 to about 25, 3 to about 40, or 3 to about 30 amino acids in length. In other embodiments are spacers that are about 5 to about 20 amino acids in length.

As such, examples of targeted CE-7 perhydrolases may include, but are not limited to, any of the CE-7 perhydrolases having an amino acid sequence selected from the group consisting of SEQ ID NOs 2, 4, 6, 8, 10, 12, 14, 16, 18, 20, 22, 24, 26, 27, 28, 29, 30, 31, 32, 33, 34, 35, 36, 38, 40, 42, 44, 46, 48, 50, 52, 54, 56, 58, 60, 62, 64, 293, 297, 301, 303, 305, 307, 309, and 311 coupled to a peptidic component having affinity for hair. In a preferred embodiment, examples of targeted perhydrolases may include, but are not limited to, any of CE-7 perhydrolases having an amino acid sequence selected from the group consisting of SEQ ID NOs 2, 4, 6, 8, 10, 12, 14, 16, 18, 20, 22, 24, 26, 27, 28, 29, 30, 31, 32, 33, 34, 35, 36, 38, 40, 42, 44, 46, 48, 50, 52, 54, 56, 58, 60, 62, 64, 293, 297, 301, 303, 305, 307, 309, and 311 coupled to one or more body surface-binding peptides having affinity for hair (optionally through a peptide spacer).

The fusion peptide may comprise at least one previously reported to have affinity for another body surface, such as skin (SEQ ID NOs: 217-269) or nail (SEQ ID NOs: 270-271). In one embodiment, the CE-7 fusion peptide comprises at least one hair-binding peptide from the group comprising SEQ ID NOs: 65-221, 271, 290, and 291. In another embodiment, the CE-7 perhydrolase fusion peptide may include any body surface-binding peptide designed to have electrostatic attraction to the target body surface (e.g., a body surface-binding peptide engineered to electrostatically bind to the target body surface).

In another embodiment, examples of targeted CE-7 perhydrolases may include, but are not limited to SEQ ID NOs 288, 289, 294, 295, 317, 319, and 321.

### Peptides Having Affinity for a Body Surface

Single chain peptides lacking an immunoglobulin fold that are capable of binding to at least one body surface are referred to as “body surface-binding peptides” (BSBPs) and may include, for example, peptides that bind to hair, skin, or nail. Peptides that have been identified to bind to at least human hair are also referred to as “hair-binding peptides (HBP).” Peptides that have been identified to bind to at least human skin are also referred to as “skin-binding peptides (SBP).” Peptides that have been identified to bind to at least human nail are also referred to as “nail-binding peptides (NBP).” Short single chain body surface-binding peptides may be empirically generated (e.g., positively charged polypeptides targeted to negatively charged surfaces) or generated using biopanning against a target body surface.

Short peptides having strong affinity for various body surfaces have been reported (U.S. Pat. Nos. 7,220,405; 7,309,482; 7,285,264 and 7,807,141; U.S. Patent Application Publication Nos. 2005-0226839; 2007-0196305; 2006-0199206; 2007-0065387; 2008-0107614; 2007-0110686; 2006-0073111; 2010-0158846 and 2010-0158847; and published PCT applications WO2008/054746; WO2004/048399, and WO2008/073368). The body surface-binding peptides have been used to construct peptide-based reagents capable of binding benefit agents to a target body surface. However, the use of these peptides to couple an active perhydrolase to the target body surface (i.e., “targeted perhydrolases”) for the production of a peracid benefit agent has not been described.

A non-limiting list of body surface-binding peptides having affinity for at least one body surface are provided herein including those having affinity for hair (hair-binding peptides having an amino acid sequence selected from the group consisting of SEQ ID NOs: 65-221, 271, 290, and 291), skin (skin-binding peptides comprise an amino acid sequence selected from the group consisting of SEQ ID NOs: 217-269), and nail (nail-binding peptides comprise an amino acid sequence selected from the group consisting of SEQ ID NOs: 270-271). In some embodiments, body surface-binding domains are comprised of body surface-binding peptides that are up to about 60 amino acids in length. In one embodiment, the body surface-binding peptides are 5 to 60 amino acids in length. In other embodiments, body surface-binding peptides are 7 to 50 amino acids in length or 7 to 30 amino acids in length. In still other embodiments are those body surface-binding peptides that are 7 to 27 amino acids in length.

While fusion peptides comprising body surface-binding peptides comprising a single hair-, skin-, nail-binding peptides are certain embodiments of the invention, in other embodiments of the invention, it may be advantageous to use multiple body surface-binding peptides. The inclusion of multiple, i.e., two or more, body surface-binding peptides can provide a peptidic component that is, for example, even more durable than those binding elements including a single body surface-binding. In some embodiments, the body surface-binding domains includes from 2 to about 50 or 2 to about 25 body surface-binding peptides. Other embodiments include those body surface-binding domains including 2 to about 10 or 2 to 5 body surface-binding peptides.

Multiple binding elements (i.e., body surface-binding peptides or body surface-binding domains) can be linked directly together or they can be linked together using peptide spacers. Certain peptide spacers are from 1 to 100 or 1 to 50 amino acids in length. In some embodiments, the peptide spacers are about 1 to about 25, 3 to about 40, or 3 to about 30 amino acids in length. In other embodiments are spacers that are about 5 to about 20 amino acids in length.

Body surface-binding domains, and the shorter body surface-binding peptides of which they are comprised, can be identified using any number of methods known to those skilled in the art, including, for example, any known biopanning techniques such as phage display, bacterial display, yeast display, ribosome display, mRNA display, and combinations thereof. Typically a random or substantially random (in the event bias exists) library of peptides is biopanned against the target body surface to identify peptides within the library having affinity for the target body surface.

The generation of random libraries of peptides is well known and may be accomplished by a variety of techniques including, bacterial display (Kemp, D. J.; Proc. Natl. Acad. Sci. USA 78(7):4520-4524 (1981), and Helfman et al., Proc. Natl. Acad. Sci. USA 80(1):31-35, (1983)), yeast display (Chien et al., Proc Nati Aced Sci USA 88(21):9578-82 (1991)), combinatorial solid phase peptide synthesis (U.S. Pat. No. 5,449,754, U.S. Pat. No. 5,480,971, U.S. Pat. No. 5,585,275, U.S. Pat. No. 5,639,603), and phage display technology (U.S. Pat. No. 5,223,409, U.S. Pat. No. 5,403,484, U.S. Pat. No. 5,571,698, U.S. Pat. No. 5,837,500); ribosome display (U.S. Pat. No. 5,643,768; U.S. Pat. No. 5,658,754; and U.S. Pat. No. 7,074,557), and mRNA display technology (PROFUSION™, see U.S. Pat. Nos. 6,258,558; 6,518,018; 6,281,344; 6,214,553; 6,261,804; 6,207,446; 6,846,655; 6,312,927; 6,602,685; 6,416,950; 6,429,300; 7,078,197; and 6,436,665).

### Binding Affinity

The peptidic component having affinity for the body surface comprises a binding affinity for human hair, skin, or nail or of 10⁻⁵ molar (M) or less. In certain embodiments, the peptidic component is one or more body surface-binding peptides and/or binding donnain(s) having a binding affinity for human hair, skin, or nail of 10⁻⁵ molar (M) or less. In some embodiments, the binding peptides or domains will have a binding affinity value of 10⁻⁵ M or less in the presence of at least about 50-500 mM salt. The term “binding affinity” refers to the strength of the interaction of a binding peptide with its respective substrate, in this case, human hair, skin, or nail. Binding affinity can be defined or measured in terms of the binding peptide's dissociation constant (“K_{D}”), or “MB₅₀.”

“K_{D}” corresponds to the concentration of peptide at which the binding site on the target is half occupied, i.e., when the concentration of target with peptide bound (bound target material) equals the concentration of target with no peptide bound. The smaller the dissociation constant, the more tightly the peptide is bound. For example, a peptide with a nanomolar (nM) dissociation constant binds more tightly than a peptide with a micromolar (μM) dissociation constant. Certain embodiments of the invention will have a K_{D} value of 10⁻⁵ or less.

“MB₅₀” refers to the concentration of the binding peptide that gives a signal that is 50% of the maximum signal obtained in an ELISA-based binding assay. See, e.g., Example 3 of U.S. Patent Application Publication 2005/022683; hereby incorporated by reference. The MB₅₀ provides an indication of the strength of the binding interaction or affinity of the components of the complex. The lower the value of MB₅₀, the stronger, i.e., “better,” the interaction of the peptide with its corresponding substrate. For example, a peptide with a nanomolar (nM) MB₅₀ binds more tightly than a peptide with a micromolar (μM) MB₅₀. Certain embodiments of the invention will have a MB₅₀ value of 10⁻⁵ M or less.

In some embodiments, the peptidic component having affinity for a body surface may have a binding affinity, as measured by K_{D} or MB₅₀ values, of less than or equal to about 10⁻⁵ M, less than or equal to about 10⁻⁶ M, less than or equal to about 10⁻⁷ M, less than or equal to about 10⁻⁸ M, less than or equal to about 10⁻⁹ M, or less than or equal to about 10⁻¹⁰ M.

In some embodiments, the body surface-binding peptides and/or body surface-binding domains may have a binding affinity, as measured by K_{D} or MB₅₀ values, of less than or equal to about 10⁻⁵ M, less than or equal to about 10⁻⁶ M, less than or equal to about 10⁻⁷ M, less than or equal to about 10⁻⁸ M, less than or equal to about 10⁻⁹ M, or less than or equal to about 10⁻¹⁰ M.

As used herein, the term “strong affinity” will refer to a binding affinity having a K_{D} or MB₅₀ value of less than or equal to about 10⁻⁵ M, preferably less than or equal to about 10⁻⁶ M, more preferably less than or equal to about 10⁻⁷ M, more preferably less than or equal to about 10⁻⁸ M, less than or equal to about 10⁻⁹ M, or most preferably less than or equal to about 10⁻¹⁰ M.

### Multicomponent Peroxycarboxylic Acid Generation Systems

The design of systems and means for separating and combining multiple active components generally will depend upon the physical form of the individual reaction components. For example, multiple active fluids (liquid-liquid) systems typically use multi-chamber dispenser bottles or two-phase systems (e.g., U.S. Patent Application Publication No. 2005/0139608; U.S. Pat. No. 5,398,846; U.S. Pat. No. 5,624,634; U.S. Pat. No. 6,391,840; E.P. Patent 0807156B1; U.S. Patent Application. Pub. No. 2005/0008526; and PCT Publication No. WO 00/61713) such as found in some bleaching applications wherein the desired bleaching agent is produced upon mixing the reactive fluids. Other forms of multicomponent systems used to generate peroxycarboxylic acid may include, but are not limited to, those designed for one or more solid components or combinations of solid-liquid components, such as powders (e.g., U.S. Pat. No. 5,116,575), multi-layered tablets (e.g., U.S. Pat. No. 6,210,639), water dissolvable packets having multiple compartments (e.g., U.S. Pat. No. 6,995,125) and solid agglomerates that react upon the addition of water (e.g., U.S. Pat. No. 6,319,888). The individual components should be safe to handle and stable for extended periods of time (i.e., as measured by the concentration of peroxycarboxylic acid produced upon mixing). In one embodiment, the storage stability of a multi-component enzymatic peroxycarboxylic acid generation system may be measured in terms of enzyme catalyst stability. In another embodiment, the storage stability of the multi-component system is measured in terms of both enzyme catalyst stability and substrate (e.g., the carboyxlic acid ester) stability.

Personal care products comprising a multi-component peroxycarboxylic acid generation formulation are provided herein that use an enzyme catalyst to rapidly produce an aqueous peracid solution having a desired peroxycarboxylic acid concentration. The mixing may occur immediately prior to use and/or at the site (in situ) of application. In one embodiment, the personal care product formulation will be comprised of at least two components that remain separated until use. Mixing of the components rapidly forms an aqueous peracid solution. Each component is designed so that the resulting aqueous peracid solution comprises an efficacious peracid concentration suitable for the intended end use (e.g., peracid-based depilation, peracid-based reduction in hair tensile strength, peracid-enhanced hair removal for use with other depilatory products (such as thioglycolate-based hair removal products), hair bleaching, hair dye pretreatment (oxidative hair dyes), hair curling, hair conditioning, skin whitening, skin bleaching, skin conditioning, reducing the appearance of skin wrinkles, skin rejuvenation, reducing dermal adhesions, reducing or eliminating body odors, nail bleaching, or nail disinfecting. The composition of the individual components should be designed to (1) provide extended storage stability and/or (2) provide the ability to enhance formation of a suitable aqueous reaction formulation comprised of peroxycarboxylic acid.

The multi-component formulation may be comprised of at least two substantially liquid components. In one embodiment, the multi-component formulation may be a two component formulation comprises a first liquid component and a second liquid component. The use of the terms “first” or “second” liquid component is relative provided that two different liquid components comprising the specified ingredients remain separated until use. At a minimum, the multi-component peroxycarboxylic acid formulation comprises (1) at least one enzyme catalyst having perhydrolysis activity, (2) a carboxylic acid ester substrate, and (3) a source of peroxygen and water wherein the formulation enzymatically produces the desired peracid upon combining the components.

The type and amount of the various ingredients used within two component formulation should to be carefully selected and balanced to provide (1) storage stability of each component, including the perhydrolysis activity of the enzyme catalyst and the stability/reactivity of each substrate, and (2) physical characteristics that enhance solubility and/or the ability to effectively form the desired aqueous peroxycarboxylic acid solution (e.g., ingredients that enhance the solubility of the ester substrate in the aqueous reaction mixture and/or ingredients that modify the viscosity and/concentration of at least one of the liquid components [i.e., at least one cosolvent that does not have a significant, adverse effect on the enzymatic perhydrolysis activity]).

Various methods to improve the performance and/or catalyst stability of enzymatic peracid generation systems have been disclosed. U.S. Patent Application Publication Nos. 2010-0048448, 2010-0086534, 2010-0086535.

The present hair care product comprises a two compositions that remain separated until use. The first composition is a non-aqueous composition comprising a mixture of:
1) at least one substrate selected from the group consisting of:
   i) esters having the structure

[X]ₘR₅
wherein X=an ester group of the formula R₆C(O)O
R₆=C1 to C7 linear, branched or cyclic hydrocarbyl moiety, optionally substituted with hydroxyl groups or C1 to C4 alkoxy groups, wherein R₆ optionally comprises one or more ether linkages for R₆=C2 to C7;
R₅=a C1 to C6 linear, branched, or cyclic hydrocarbyl moiety or a five-membered cyclic heteroaromatic moiety or six-membered cyclic aromatic or heteroaromatic moiety optionally substituted with hydroxyl groups;
wherein each carbon atom in R₅ individually comprises no more than one hydroxyl group or no more than one ester group or carboxylic acid group;
wherein R₅ optionally comprises one or more ether linkages;
m is an integer ranging from 1 to the number of carbon atoms in R₅; and
wherein said esters have a solubility in water of at least 5 ppm at 25° C.;
   ii) glycerides having the structure

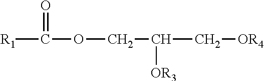

wherein R₁=C1 to C7 straight chain or branched chain alkyl optionally substituted with an hydroxyl or a C1 to C4 alkoxy group and R₃ and R₄ are individually H or R₁C(O);
   iii) one or more esters of the formula

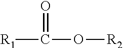

wherein R₁ is a C1 to C7 straight chain or branched chain alkyl optionally substituted with an hydroxyl or a C1 to C4 alkoxy group and R₂ is a C1 to 010 straight chain or branched chain alkyl, alkenyl, alkynyl, aryl, alkylaryl, alkylheteroaryl, heteroaryl, (CH₂CH₂O)ₙ, or (CH₂CH(CH₃)−O)ₙH and n is 1 to 10; and
iv) acetylated saccharides selected from the group consisting of acetylated monosaccharides, acetylated disaccharides, and acetylated polysaccharides;
2) a solid source of peroxygen such as perborate, percarbonate or a combination thereof; and
3) an optional organic cosolvent.
The second component is an aqueous composition comprising:
1) an enzyme catalyst having perhydrolytic activity;
2) at least one buffer; wherein the aqueous composition comprises a pH of at least 4.

The non-aqueous composition and the aqueous compositions remain separated prior to use and wherein an enzymatically generated peracid is produced upon combining the non-aqueous and aqueous compositions.

The type and amount of buffer(s) incorporated in the aqueous composition are chosen such that the pH of the aqueous composition (prior to use) is maintained at a pH of at least 4, preferably in a range from about 4 to about 9. The reaction components are selected such that the resulting reaction mixture obtained upon combing the non-aqueous and the aqueous compositions comprises a pH wherein the enzyme catalyst has perhydrolytic activity and whereby at least on peracid is produced.

The arrangement of the components in the two compositions described herein exhibit storage stability for both the enzyme catalyst (as measured by enzyme activity observed upon initiating the reaction) and substrates (the carboxylic acid ester and the source of peroxygen do no significantly decompose during storage).

As used herein, “substantially stable” means that the storage stability of the component in question retains activity (such as enzyme catalyst activity) or does not significantly change in composition (e.g., the concentration substrate does not substantially change during storage) during storage (prior to use). In one embodiment, the storage conditions comprises storage of the composition at 25° C. for at least 14 days; wherein at least 70%, preferably at least 80%, more preferable at least 90%, even more preferably at least 95%, even more preferably at least 99%, and most preferably about 100% of the original activity (e.g., enzyme catalyst activity) and original substrate concentration (e.g. the carboxylic acid ester substrate) are maintained relative to the activity/concentrations obtained upon creating the compositions. Means to measure catalyst stability and substrate stability are described herein.

### Enzyme Powders

In some embodiments, the personal care compositions may use an enzyme catalyst in form of a stabilized enzyme powder. Methods to make and stabilize formulations comprising an enzyme powder are described in U.S. Patent Application Publication Nos. 2010-0086534 and 2010-0086535.

In one embodiment, the enzyme may be in the enzyme powder in an amount in a range of from about 5 weight percent (wt %) to about 75 wt % based on the dry weight of the enzyme powder. A preferred weight percent range of the enzyme in the enzyme powder/spray-dried mixture is from about 10 wt % to 50 wt %, and a more preferred weight percent range of the enzyme in the enzyme powder/spray-dried mixture is from about 20 wt % to 33 wt %

In one embodiment, the enzyme powder may further comprise an excipient. In one aspect, the excipient is provided in an amount in a range of from about 95 wt % to about 25 wt % based on the dry weight of the enzyme powder. A preferred wt % range of excipient in the enzyme powder is from about 90 wt % to 50 wt %, and a more preferred wt % range of excipient in the enzyme powder is from about 80 wt % to 67 wt %_{.}

In one embodiment, the excipient used to prepare an enzyme powder may be an oligosaccharide excipient. In one embodiment, the oligosaccharide excipient has a number average molecular weight of at least about 1250 and a weight average molecular weight of at least about 9000. In some embodiments, the oligosaccharide excipient has a number average molecular weight of at least about 1700 and a weight average molecular weight of at least about 15000. Specific oligosaccharides may include, but are not limited to, maltodextrin, xylan, mannan, fucoidan, galactomannan, chitosan, raffinose, stachyose, pectin, insulin, levan, graminan, amylopectin, sucrose, lactulose, lactose, maltose, trehalose, cellobiose, nigerotriose, maltotriose, melezitose, maltotriulose, raffinose, kestose, and mixtures thereof. In a preferred embodiment, the oligosaccharide excipient is maltodextrin. Oligosaccharide-based excipients may also include, but are not limited to, water-soluble non-ionic cellulose ethers, such as hydroxymethyl-cellulose and hydroxypropylmethylcellulose, and mixtures thereof. In yet a further embodiment, the excipient may be selected from, but not limited to, one or more of the following compounds: trehalose, lactose, sucrose, mannitol, sorbitol, glucose, cellobiose, α-cyclodextrin, and carboxymethylcellulose.

The formulations may comprise at least one optional surfactant, where the presence of at least one surfactant is preferred. Surfactants may include, but are not limited to, ionic and nonionic surfactants or wetting agents, such as ethoxylated castor oil, polyglycolyzed glycerides, acetylated monoglycerides, sorbitan fatty acid esters, poloxamers, polyoxyethylene sorbitan fatty acid esters, polyoxyethylene derivatives, monoglycerides or ethoxylated derivatives thereof, diglycerides or polyoxyethylene derivatives thereof, sodium docusate, sodium laurylsulfate, cholic acid or derivatives thereof, lecithins, phospholipids, block copolymers of ethylene glycol and propylene glycol, and non-ionic organosilicones. Preferably, the surfactant is a polyoxyethylene sorbitan fatty acid ester, with polysorbate 80 being more preferred.

In one embodiment, suitable nonionic surfactants may include cetomacrogol 1000 (polyoxyethylene(20) cetyl ether), cetostearyl alcohol, cetyl alcohol, coco-betaine, cocamide DEA, cocamide MEA, cocoglycerides, coco-glucoside, decyl glucoside, glyceryl laurate, glyceryl oleate, isoceteth-20, lauryl glucoside, narrow range ethoxylates, NONIDET® P-40, nonoxynol-9, nonoxynols, NP-40, octaethylene glycol monododecyl ether, octyl glucoside, oleyl alcohol, pentaethylene glycol monododecyl ether, Poloxamer, Poloxamer 407, polyglycerol polyricinoleate, polyglyceryl-10 laurate, polysorbate, polysorbate 20, polysorbate 80, sodium coco-sulfate, sorbitan monostearate, sorbitan tristearate, stearyl alcohol, sucrose laurate, TRITON® X-100, TWEEN®-20, and TWEEN®-80.

When the formulation comprises an enzyme powder, the surfactant used to prepare the powder may be present in an amount ranging of from about 5 wt % to 0.1 wt % based on the weight of protein present in the enzyme powder, preferably from about 2 wt % to 0.5 wt % based on the weight of protein present in the enzyme powder.

The enzyme powder may additionally comprise one or more buffers (e.g., sodium and/or potassium salts of bicarbonate, citrate, acetate, phosphate, pyrophosphate, methylphosphonate, succinate, malate, fumarate, tartrate, and maleate), and an enzyme stabilizer (e.g., ethylenediaminetetraacetic acid, (1-hydroxyethylidene)bisphosphonic acid)).

Spray drying of the formulation to form the enzyme powder is carried out, for example, as described generally in Spray Drying Handbook, 5^{th} ed., K. Masters, John Wiley & Sons, Inc., NY, N.Y. (1991), and in PCT Patent Publication Nos. WO 97/41833 and WO 96/32149 to Platz, R. et al.

In general spray drying consists of bringing together a highly dispersed liquid, and a sufficient volume of hot air to produce evaporation and drying of the liquid droplets. Typically the feed is sprayed into a current of warm filtered air that evaporates the solvent and conveys the dried product to a collector. The spent air is then exhausted with the solvent. Those skilled in the art will appreciate that several different types of apparatus may be used to provide the desired product. For example, commercial spray dryers manufactured. by Buchi Ltd. (Postfach, Switzerland) or GEA Niro Corp. (Copenhagen, Denmark) will effectively produce particles of desired size. It will further be appreciated that these spray dryers, and specifically their atomizers, may be modified or customized for specialized applications, such as the simultaneous spraying of two solutions using a double nozzle technique. More specifically, a water-in-oil emulsion can be atomized from one nozzle and a solution containing an anti-adherent such as mannitol can be co-atomized from a second nozzle. In other cases it may be desirable to push the feed solution though a custom designed nozzle using a high pressure liquid chromatography (HPLC) pump. Provided that microstructures comprising the correct morphology and/or composition are produced the choice of apparatus is not critical and would be apparent to the skilled artisan in view of the teachings herein.

The temperature of both the inlet and outlet of the gas used to dry the sprayed material is such that it does not cause degradation of the enzyme in the sprayed material. Such temperatures are typically determined experimentally, although generally, the inlet temperature will range from about 50° C. to about 225° C., while the outlet temperature will range from about 30° C. to about 150° C. Preferred parameters include atomization pressures ranging from about 20-150 psi (0.14 MPa-1.03 MPa), and preferably from about 30-40 to 100 psi (0.21-0.28 MPa to 0.69 MPa). Typically the atomization pressure employed will be one of the following (MPa) 0.14, 0.21, 0.28, 0.34, 0.41, 0.48, 0.55, 0.62, 0.69, 0.76, 0.83 or above.

Suitable Reaction Conditions for the Enzyme-catalyzed Preparation of Peracids from Carboxylic Acid Esters and Hydrogen Peroxide

One or more enzymes having perhydrolytic activity may be used to generate an efficacious concentration of the desired peracid(s) in the present personal care compositions and methods. The desired peroxycarboxylic acid may be prepared by reacting carboxylic acid esters with a source of peroxygen including, but not limited to, hydrogen peroxide, sodium perborate or sodium percarbonate, in the presence of an enzyme catalyst having perhydrolysis activity.

The perhydrolytic enzyme within the targeted perhydrolase may be any perhydrolytic enzyme and may include lipases, proteases, esterases, acyl transferases, aryl esterases, carbohydrate esterases, and combinations so long as the enzyme has perhydrolytic activity for one or more of the present substrates. Examples may include, but are not limited to perhydrolytic proteases (subtilisin variant; U.S. Pat. No. 7,510,859), perhydrolytic esterases (Pseudomonas fluorescens; U.S. Pat. No. 7,384,787; SEQ ID NO: 315 [L29P variant] and SEQ ID NO: 339 [wild type]), perhydrolytic aryl esterases (Mycobacterium smegmatis; U.S. Pat. No. 7,754,460; WO2005/056782; and EP1689859 B1; SEQ ID NOs: 314 [S54V variant] and 338 [wild type]).

In one embodiment, the enzyme catalyst comprises at least one enzyme having perhydrolase activity, wherein said enzyme is structurally classified as a member of the CE-7 carbohydrate esterase family (CE-7; see Coutinho, P. M., and Henrissat, B., supra). In another embodiment, the perhydrolase catalyst is structurally classified as a cephalosporin C deacetylase. In another embodiment, the perhydrolase catalyst is structurally classified as an acetyl xylan esterase.

In one embodiment, the perhydrolase catalyst comprises an enzyme having perhydrolysis activity and a CE-7 signature motif comprising:
a) an RGQ motif that aligns with amino acid residues 118-120 of SEQ ID NO: 2;
b) a GXSQG motif that aligns with amino acid residues 179-183 of SEQ ID NO: 2; and
c) an HE motif that aligns with amino acid residues 298-299 of SEQ ID NO: 2.

In a preferred embodiment, the alignment to reference SEQ ID NO: 2 is performed using CLUSTALW.

In a further embodiment, the CE-7 signature motif additional may comprise and additional (i.e., fourth) motif defined as an LXD motif at amino acid residues 267-269 when aligned to reference sequence SEQ ID NO:2 using CLUSTALW.

In another embodiment, the perhydrolase catalyst comprises an enzyme having perhydrolase activity, said enzyme having an amino acid sequence selected from the group consisting of SEQ ID NOs: 2, 4, 6, 8, 10, 12, 14, 16, 18, 20, 22, 24, 26, 27, 28, 29, 30, 31, 32, 33, 34, 35, 36, 38, 40, 42, 44, 46, 48, 50, 52, 54, 56, 58, 60, 62, 64, 293, 297, 299, 301, 303, 305, 307, 309, and 311.

In another embodiment, the perhydrolase catalyst comprises an enzyme having perhydrolase activity, said enzyme having an amino acid sequence selected from the group consisting of SEQ ID NOs: 2, 4, 6, 8, 10, 12, 14, 16, 18, 20, 22, 24, 26, 27, 28, 29, 30, 31, 32, 33, 34, 35, 36, 38, 40, 42, 44, 46, 48, 50, 52, 54, 56, 58, 60, 62, 64, 293, 297, 299, 301, 303, 305, 307, 309, and 311 wherein said enzyme may have one or more additions, deletions, or substitutions so long as the signature motif is conserved and perhydrolase activity is retained.

As described above, the CE-7 perhydrolase may be a fusion protein having a first portion comprising CE-7 perhydrolase and a second portion comprising a peptidic component having affinity for a target body surface such at that perhydrolase is “targeted” to the desired body surface. In one embodiment, any CE-7 perhydrolase (as defined by the presence of the CE-7 signature motifs) may be fused to any peptidic component/binding element capable of targeting the enzyme to a body surface. In one aspect, the peptidic component having affinity for hair may include antibodies, antibody fragments (F_{ab}), as well as single chain variable fragments (scFv; a fusion of the variable regions of the heavy (V_{H}) and light chains (V_{L}) of immunoglobulins), single domain camelid antibodies, scaffold display proteins, and single chain affinity peptides lacking immunoglobulin folds. The compositions comprising antibodies, antibodies fragments and other immunoglobulin-derived binding elements, as well as large scaffold display proteins, are often not economically viable. As such, and in a preferred aspect, the peptidic component/binding element is a single chain affinity peptide lacking an immunoglobulin fold and/or immunoglobulin domain. Short single chain body surface-binding peptides may be empirically generated (e.g., positively charged polypeptides targeted to negatively charged surfaces) or generated using biopanning against a target body surface. Methods to identify/obtain affinity peptides using any number of display techniques (e.g., phage display, yeast display, bacterial display, ribosome display, and mRNA display) are well known in the art. Individual hair-binding peptides may be coupled together, via optional spacers/linkers, to form larger binding “domains” (also referred to herein as binding “hands”) to enhance attachment/localization of the perhydrolytic enzyme to hair.

The fusion proteins may also include one or more peptide linkers/spacers separating the CE-7 perhydrolase enzyme and the hair-binding domain and/or between different hair-binding peptides (e.g., when a plurality of hair-binding peptides are coupled together to form a larger target hair-binding domain). A non-limiting list of exemplary peptide spacers are provided by the amino acid sequences of SEQ ID NOs: 290, 291, 312, and 313.

Suitable peptides having affinity for hair are described herein, supra. Methods to identify additional hair-binding peptides using any of the above “display” techniques are well known and can be used to identify additional hair-binding peptides.

Suitable carboxylic acid ester substrates may include esters having the following formula:
(a) one or more esters having the structure

[X]ₘR₅
wherein
X is an ester group of the formula R₆C(O)O;
R₆ is a C1 to C7 linear, branched or cyclic hydrocarbyl moiety, optionally substituted with a hydroxyl group or C1 to C4 alkoxy group, wherein R₆ optionally comprises one or more ether linkages where R₆ is C2 to C7;
R₅ is a C1 to C6 linear, branched, or cyclic hydrocarbyl moiety or a five-membered cyclic heteroaromatic moiety or six-membered cyclic aromatic or heteroaromatic moiety optionally substituted with a hydroxyl group; wherein each carbon atom in R₅ individually comprises no more than one hydroxyl group or no more than one ester group or carboxylic acid group, and wherein R₅ optionally comprises one or more ether linkages;
m is an integer ranging from 1 to the number of carbon atoms in R₅,
said one or more esters having solubility in water of at least 5 ppm at 25° C.; or
(b) one or more glycerides having the structure

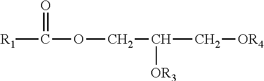

wherein R₁ is a C1 to C7 straight chain or branched chain alkyl optionally substituted with an hydroxyl or a C1 to C4 alkoxy group and R₃ and R₄ are individually H or R₁C(O); or
(c) one or more esters of the formula

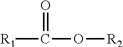

wherein R₁ is a C1 to C7 straight chain or branched chain alkyl optionally substituted with an hydroxyl or a C1 to C4 alkoxy group and R₂ is a C1 to C10 straight chain or branched chain alkyl, alkenyl, alkynyl, aryl, alkylaryl, alkylheteroaryl, heteroaryl, (CH₂CH₂O)ₙ, or (CH₂CH(CH₃)—O)ₙH and n is 1 to 10; or
(d) one or more acetylated monosaccharides, acetylated disaccharides, or acetylated polysaccharides; or
(e) any combination of (a) through (d).

Suitable substrates may also include one or more acylated saccharides selected from the group consisting of acylated mono-, di-, and polysaccharides. In another embodiment, the acylated saccharides are selected from the group consisting of acetylated xylan; fragments of acetylated xylan; acetylated xylose (such as xylose tetraacetate); acetylated glucose (such as α-D-glucose pentaacetate; β-D-glucose pentaacetate; 1-thio-β-D-glucose-2,3,4,6-tetraacetate); β-D-galactose pentaacetate; sorbitol hexaacetate; sucrose octaacetate; β-D-ribofuranose-1,2,3,5-tetraacetate; β-D-ribofuranose-1,2,3,4-tetraacetate; tri-O-acetyl-D-galactal; tri-O-acetyl-D-glucal; β-D-xylofuranose tetraacetate, α-D-glucopyranose pentaacetate; β-D-glucopyranose-1,2,3,4-tetraacetate; β-D-glucopyranose-2,3,4,6-tetraacetate; 2-acetamido-2-deoxy-1,3,4,6-tetracetyl-β-D-glucopyranose; 2-acetamido-2-deoxy-3,4,6-triacetyl-1-chloride-α-D-glucopyranose; α-D-mannopyranose pentaacetate, and acetylated cellulose. In a preferred embodiment, the acetylated saccharide is selected from the group consisting of β-D-ribofuranose-1,2,3,5-tetraacetate; tri-O-acetyl-D-galactal; tri-O-acetyl-D-glucal; sucrose octaacetate; and acetylated cellulose.

In another embodiment, additional suitable substrates may also include 5-acetoxymethyl-2-furaldehyde; 3,4-diacetoxy-1-butene; 4-acetoxybenezoic acid; vanillin acetate; propylene glycol methyl ether acetate; methyl lactate; ethyl lactate; methyl glycolate; ethyl glycolate; methyl methoxyacetate; ethyl methoxyacetate; methyl 3-hydroxybutyrate; ethyl 3-hydroxybutyrate; and triethyl 2-acetyl citrate.

In another embodiment, suitable substrates are selected from the group consisting of: monoacetin; diacetin; triacetin; monopropionin; dipropionin; tripropionin; monobutyrin; dibutyrin; tributyrin; glucose pentaacetate; xylose tetraacetate; acetylated xylan; acetylated xylan fragments; β-D-ribofuranose-1,2,3,5-tetraacetate; tri-O-acetyl-D-galactal; tri-O-acetyl-D-glucal; monoesters or diesters of 1,2-ethanediol; 1,2-propanediol; 1,3-propanediol; 1,2-butanediol; 1,3-butanediol; 2,3-butanediol; 1,4-butanediol; 1,2-pentanediol; 2,5-pentanediol; 1,5-pentanediol; 1,6-pentanediol; 1,2-hexanediol; 2,5-hexanediol; 1,6-hexanediol; and mixtures thereof. In another embodiment, the substrate is a C1 to C6 polyol comprising one or more ester groups. In a preferred embodiment, one or more of the hydroxyl groups on the C1 to C6 polyol are substituted with one or more acetoxy groups (such as 1,3-propanediol diacetate; 1,2-propanediol diacetate; 1,4-butanediol diacetate; 1,5-pentanediol diacetate, etc.). In a further embodiment, the substrate is propylene glycol diacetate (PGDA), ethylene glycol diacetate (EGDA), or a mixture thereof.

In a further embodiment, suitable substrates are selected from the group consisting of monoacetin, diacetin, triacetin, monopropionin, dipropionin, tripropionin, monobutyrin, dibutyrin, and tributyrin. In yet another aspect, the substrate is selected from the group consisting of diacetin and triacetin. In a most preferred embodiment, the suitable substrate comprises triacetin.

In a preferred embodiment, the carboxylic acid ester is a liquid substrate selected from the group consisting of monoacetin, diacetin, triacetin, and combinations (i.e., mixtures) thereof. The carboxylic acid ester is present in the reaction formulation at a concentration sufficient to produce the desired concentration of peroxycarboxylic acid upon enzyme-catalyzed perhydrolysis. The carboxylic acid ester need not be completely soluble in the reaction formulation, but has sufficient solubility to permit conversion of the ester by the perhydrolase catalyst to the corresponding peroxycarboxylic acid. The carboxylic acid ester is present in the reaction formulation at a concentration of 0.05 wt % to 40 wt % of the reaction formulation, preferably at a concentration of 0.1 wt % to 20 wt % of the reaction formulation, and more preferably at a concentration of 0.5 wt % to 10 wt % of the reaction formulation.

The peroxygen source may include, but is not limited to, hydrogen peroxide, hydrogen peroxide adducts (e.g., urea-hydrogen peroxide adduct (carbamide peroxide)) perborate salts and percarbonate salts. The concentration of peroxygen compound in the reaction formulation may range from 0.0033 wt % to about 50 wt %, preferably from 0.033 wt % to about 40 wt %, more preferably from 0.1 wt % to about 30 wt %.

The peroxygen source (i.e., hydrogen peroxide) may also be generated enzymatically using enzyme capable of producing and effective amount of hydrogen peroxide. For example, various oxidases can be used in the present compositions and methods to produce an effective amount of hydrogen peroxide including, but not limited to glucose oxidase, lactose oxidases, carbohydrate oxidase, alcohol oxidase, ethylene glycol oxidase, glycerol oxidase, and amino acid oxidase.

Many perhydrolase catalysts (whole cells, permeabilized whole cells, and partially purified whole cell extracts) have been reported to have catalase activity (EC 1.11.1.6). Catalases catalyze the conversion of hydrogen peroxide into oxygen and water. In one aspect, the perhydrolysis catalyst lacks catalase activity. In another aspect, a catalase inhibitor may be added to the reaction formulation. One of skill in the art can adjust the concentration of catalase inhibitor as needed. The concentration of the catalase inhibitor typically ranges from 0.1 mM to about 1 M; preferably about 1 mM to about 50 mM; more preferably from about 1 mM to about 20 mM.

In another embodiment, the enzyme catalyst lacks significant catalase activity or may be engineered to decrease or eliminate catalase activity. The catalase activity in a host cell can be down-regulated or eliminated by disrupting expression of the gene(s) responsible for the catalase activity using well known techniques including, but not limited to, transposon mutagenesis, RNA antisense expression, targeted mutagenesis, and random mutagenesis. In a preferred embodiment, the gene(s) encoding the endogenous catalase activity are down-regulated or disrupted (i.e., knocked-out). As used herein, a “disrupted” gene is one where the activity and/or function of the protein encoded by the modified gene is no longer present. Means to disrupt a gene are well-known in the art and may include, but are not limited to, insertions, deletions, or mutations to the gene so long as the activity and/or function of the corresponding protein is no longer present. In a further preferred embodiment, the production host is an E. coli production host comprising a disrupted catalase gene selected from the group consisting of katG and katE (see U.S. Patent Application Publication No. 2008-0176299). In another embodiment, the production host is an E. coli strain comprising a down-regulation and/or disruption in both katG and a katE catalase genes.

The concentration of the catalyst in the aqueous reaction formulation depends on the specific catalytic activity of the catalyst, and is chosen to obtain the desired rate of reaction. The weight of catalyst in perhydrolysis reactions typically ranges from 0.0001 mg to 10 mg per mL of total reaction volume, preferably from 0.001 mg to 2.0 mg per mL. The catalyst may also be immobilized on a soluble or insoluble support using methods well-known to those skilled in the art; see for example, Immobilization of Enzymes and Cells; Gordon F. Bickerstaff, Editor; Humana Press, Totowa, N.J., USA; 1997. The use of immobilized catalysts permits the recovery and reuse of the catalyst in subsequent reactions. The enzyme catalyst may be in the form of whole microbial cells, permeabilized microbial cells, microbial cell extracts, partially-purified or purified enzymes, and mixtures thereof.

In one aspect, the concentration of peroxycarboxylic acid generated by the combination of chemical perhydrolysis and enzymatic perhydrolysis of the carboxylic acid ester is sufficient to provide an effective concentration of peroxycarboxylic acid for the chosen personal care application. In another aspect, the present methods provide combinations of enzymes and enzyme substrates to produce the desired effective concentration of peroxycarboxylic acid, where, in the absence of added enzyme, there is a significantly lower concentration of peroxycarboxylic acid produced. Although there may in some cases be substantial chemical perhydrolysis of the enzyme substrate by direct chemical reaction of inorganic peroxide with the enzyme substrate, there may not be a sufficient concentration of peroxycarboxylic acid generated to provide an effective concentration of peroxycarboxylic acid in the desired applications, and a significant increase in total peroxycarboxylic acid concentration is achieved by the addition of an appropriate perhydrolase catalyst to the reaction formulation.

The concentration of peroxycarboxylic acid generated (e.g. peracetic acid) by the perhydrolysis of at least one carboxylic acid ester is at least about 0.1 ppm, preferably at least 0.5 ppm, 1 ppm, 5 ppm, 10 ppm, 20 ppm, 100 ppm, 200 ppm, 300 ppm, 500 ppm, 700 ppm, 1000 ppm, 2000 ppm, 5000 ppm or 10,000 ppm of peracid within 60 minutes, preferably within 30 minutes, of initiating the perhydrolysis reaction. The product formulation comprising the peroxycarboxylic acid may be optionally diluted with water, or a solution predominantly comprised of water, to produce a formulation with the desired lower concentration of peroxycarboxylic acid base on the target application. Clearly one of skill in the art can adjust the reaction components and/or dilution amounts to achieve the desired peracid concentration for the chosen personal care product.

The peracid formed in accordance with the processes describe herein is used in a personal care product/application wherein the peracid is contacted with a target body surface to provide a peracid-based benefit, such as hair removal (a peracid depilatory agent), decrease hair tensile strength, a hair pretreatment used to enhance other depilatory products (such as thioglycolate-based hair removal products), hair bleaching, hair dye pretreatment (oxidative hair dyes), hair curling, hair conditioning, skin whitening, skin bleaching, skin conditioning, reducing the appearance of skin wrinkles, skin rejuvenation, reducing dermal adhesions, reducing or eliminating body odors, nail bleaching, or nail disinfecting. In one embodiment, the process to produce a peracid for a target body surface is conducted in situ.

The temperature of the reaction may be chosen to control both the reaction rate and the stability of the enzyme catalyst activity. Clearly for certain personal care applications the temperature of the target body surface may be the temperature of the reaction. The temperature of the reaction may range from just above the freezing point of the reaction formulation (approximately 0° C.) to about 95° C., with a preferred range of 5° C. to about 75° C., and a more preferred range of reaction temperature of from about 5° C. to about 55° C.

The pH of the final reaction formulation containing peroxycarboxylic acid is from about 2 to about 9, preferably from about 3 to about 8, more preferably from about 5 to about 8, even more preferably about 5.5 to about 8, and yet even more preferably about 6.0 to about 7.5. The pH of the reaction, and of the final reaction formulation, may optionally be controlled by the addition of a suitable buffer including, but not limited to, phosphate, pyrophosphate, bicarbonate, acetate, or citrate. The concentration of buffer, when employed, is typically from 0.1 mM to 1.0 M, preferably from 1 mM to 300 mM, most preferably from 10 mM to 100 mM.

In another aspect, the enzymatic perhydrolysis reaction formulation may contain an organic solvent that acts as a dispersant to enhance the rate of dissolution of the carboxylic acid ester in the reaction formulation. Such solvents include, but are not limited to, propylene glycol methyl ether, acetone, cyclohexanone, diethylene glycol butyl ether, tripropylene glycol methyl ether, diethylene glycol methyl ether, propylene glycol butyl ether, dipropylene glycol methyl ether, cyclohexanol, benzyl alcohol, isopropanol, ethanol, propylene glycol, and mixtures thereof.

Single Step Vs. Multi-Step Application Methods

Typically the minimum set of reaction components to enzymatically produce a peracid benefit agent will include (1) at least one enzyme having perhydrolytic activity as described herein, such as a CE-7 perhydrolase (optionally in the form of a targeted fusion protein), (2) at least one suitable carboyxlic acid ester substrate, and (3) a source of peroxygen.

The peracid-generating reaction components of the personal care composition may remain separated until use. In one embodiment, the peracid-generating components are combined and then contacted with the target body surface whereby the resulting peracid-based benefit agent provides a benefit to the body surface. The components may be combined and then contacted with the target body surface or may be combined on the targeted body surface. In one embodiment, the peracid-generating components are combined such that the peracid is produced in situ.

A multi-step application may also be used. One or two of the individual components of the peracid-generating system (i.e., a sequential application on the body surface of at least one of the three basic reaction components) composition may be contacted with hair prior to applying the remaining components required for enzymatic peracid production. In one embodiment, the perhydrolytic enzyme is contacted with the hair prior to contacting the hair with the carboyxlic acid ester substrate and/or the source of peroxygen (i.e., a “two-step application”). In one embodiment, the enzyme having perhydrolytic activity is a targeted perhydrolase that is applied to hair prior to combining the remaining components necessary for enzymatic peracid production.

In a preferred embodiment, the enzyme having perhydrolytic activity is a “targeted CE-7 perhydrolase” CE-7 fusion protein) that is applied to hair prior to combining the remaining components necessary for enzymatic peracid production (i.e., a two-step application method). The targeted perhydrolase is contacted with the hair under suitable conditions to promote non-covalent bonding of the fusion protein to the hair surface. An optional rinsing step may be used to remove excess and/or unbound fusion protein prior to combining the remaining reaction components.

In another embodiment, the carboxylic acid ester substrate and the source of peroxygen (e.g., a non-aqueous suspension of solid source of peroxygen in the carboxylic acid ester and one or more optional cosolvent) are applied to the hair prior to the addition of the perhydrolytic enzyme (optionally in the form of a fusion protein targeted to hair).

In yet another embodiment, any of the compositions or methods described herein can be incorporated into a kit for practicing the invention. The kits may comprise materials and reagents to facilitate enzymatic production of peracid. An exemplary kit comprises a first container or compartment comprising (1) a composition that is non-aqueous having a solid source of peroxygen, a carboxylic acid ester substrate, and optionally one or more organic cosolvents and (2) a second container or compartment having an aqueous composition comprising the enzyme catalyst having perhydrolytic activity and at least one buffer, wherein the enzyme catalyst can be optionally targeted to hair or a body surface comprising hair. Other kit components may include, without limitation, one or more of the following: sample tubes, solid supports, instruction material, and other solutions or other chemical reagents useful in enzymatically producing peracids, such as acceptable components or carriers.

### Dermatologically Acceptable Components/Carriers/Medium

The compositions and methods described herein may further comprise one or more dermatologically or cosmetically acceptable components known or otherwise effective for use in hair care or other personal care products, provided that the optional components are physically and chemically compatible with the essential components described herein, or do not otherwise unduly impair product stability, aesthetics, or performance. Non-limiting examples of such optional components are disclosed in International Cosmetic Ingredient Dictionary, Ninth Edition, 2002, and CTFA Cosmetic Ingredient Handbook, Tenth Edition, 2004.

In one embodiment, the dermatologically acceptable carrier may comprise from about 10 wt % to about 99.9 wt %, alternatively from about 50 wt % to about 95 wt %, and alternatively from about 75 wt % to about 95 wt %, of a dermatologically acceptable carrier. Carriers suitable for use with the connposition(s) may include, for example, those used in the formulation of hair sprays, mousses, tonics, gels, skin moisturizers, lotions, and leave-on conditioners. The carrier may comprise water; organic oils; silicones such as volatile silicones, amino or non-amino silicone gums or oils, and mixtures thereof; mineral oils; plant oils such as olive oil, castor oil, rapeseed oil, coconut oil, wheatgerm oil, sweet almond oil, avocado oil, macadamia oil, apricot oil, safflower oil, candlenut oil, false flax oil, tamanu oil, lemon oil and mixtures thereof; waxes; and organic compounds such as C₂-C₁₀ alkanes, acetone, methyl ethyl ketone, volatile organic C₁-C₁₂ alcohols, esters (with the understanding that the choice of ester(s) may be dependent on whether or not it may act as a carboxylic acid ester substrates for the perhydrolases) of C₁-C₂₀ acids and of C₁-C₈ alcohols such as methyl acetate, butyl acetate, ethyl acetate, and isopropyl myristate, dimethoxyethane, diethoxyethane, C₁₀-C₃₀ fatty alcohols such as lauryl alcohol, cetyl alcohol, stearyl alcohol, and behenyl alcohol; C₁₀-C₃₀ fatty acids such as lauric acid and stearic acid; C₁₀-C₃₀ fatty amides such as lauric diethanolamide; C₁₀-C₃₀ fatty alkyl esters such as C₁₀-C₃₀ fatty alkyl benzoates; hydroxypropylcellulose, and mixtures thereof. In one embodiment, the carrier comprises water, fatty alcohols, volatile organic alcohols, and mixtures thereof.

The composition(s) of the present invention further may comprise from about 0.1% to about 10%, and alternatively from about 0.2% to about 5.0%, of a gelling agent to help provide the desired viscosity to the composition(s). Non-limiting examples of suitable optional gelling agents include crosslinked carboxylic acid polymers; unneutralized crosslinked carboxylic acid polymers; unneutralized modified crosslinked carboxylic acid polymers; crosslinked ethylene/maleic anhydride copolymers; unneutralized crosslinked ethylene/maleic anhydride copolymers (e.g., EMA 81 commercially available from Monsanto); unneutralized crosslinked alkyl ether/acrylate copolymers (e.g., SALCARE™ SC90 commercially available from Allied Colloids); unneutralized crosslinked copolymers of sodium polyacrylate, mineral oil, and PEG-1 trideceth-6 (e.g., SALCARE™ SC91 commercially available from Allied Colloids); unneutralized crosslinked copolymers of methyl vinyl ether and maleic anhydride (e.g., STABILEZE™ QM-PVM/MA copolymer commercially available from International Specialty Products); hydrophobically modified nonionic cellulose polymers; hydrophobically modified ethoxylate urethane polymers (e.g., UCARE™ Polyphobe Series of alkali swellable polymers commercially available from Union Carbide); and combinations thereof. In this context, the term “unneutralized” means that the optional polymer and copolymer gelling agent materials contain unneutralized acid monomers. Preferred gelling agents include water-soluble unneutralized crosslinked ethylene/maleic anhydride copolymers, water-soluble unneutralized crosslinked carboxylic acid polymers, water-soluble hydrophobically modified nonionic cellulose polymers and surfactant/fatty alcohol gel networks such as those suitable for use in hair conditioning products.

### Hair Care Compositions

The peracid generation components can be incorporated into hair care compositions and products to generate an efficacious concentration of at least one peracid. The perhydrolase used to generate the desired amount of peracid may be used in the form of a fusion protein where the first portion of the fusion protein comprises the perhydrolase a second portion having affinity for hair.

The peracid produced provides a benefit to hair (i.e., a “peracid-based benefit agent”). The peracid may be used as a depilatory agent, a hair treatment agent to reduce the tensile strength of hair, a hair pretreatment agent used to enhance the performance of other depilatory products (such as thioglycolate-based hair removal products), a hair bleaching agent, a hair dye pretreatment agent, a hair curling/styling agent, and as a component in hair conditioning products.

In addition to the peracid-based benefit agent, hair care products and formulations may also include any number of additional components commonly found in hair care products. The additional components may help to improve the appearance, texture, color, and sheen of hair as well as increasing hair body or suppleness.

Hair conditioning agents are well known in the art, see for example Green et al. (WO 0107009), and are available commercially from various sources. Suitable examples of hair conditioning agents include, but are not limited to, cationic polymers, such as cationized guar gum, diallyl quaternary ammonium salt/acrylamide copolymers, quaternized polyvinylpyrrolidone and derivatives thereof, and various polyquaternium-compounds; cationic surfactants, such as stearalkonium chloride, centrimonium chloride, and sapamin hydrochloride; fatty alcohols, such as behenyl alcohol; fatty amines, such as stearyl amine; waxes; esters; nonionic polymers, such as polyvinylpyrrolidone, polyvinyl alcohol, and polyethylene glycol; silicones; siloxanes, such as decamethylcyclopentasiloxane; polymer emulsions, such as amodimethicone; and nanoparticles, such as silica nanoparticles and polymer nanoparticles.

The hair care products may also include additional components typically found in cosmetically acceptable media. Non-limiting examples of such components are disclosed in International Cosmetic Ingredient Dictionary, Ninth Edition, 2002, and CTFA Cosmetic Ingredient Handbook, Tenth Edition, 2004. A non-limiting list of components often included in a cosmetically acceptable medium for hair care are also described by Philippe et al. in U.S. Pat. No. 6,280,747, and by Omura et al. in U.S. Pat. No. 6,139,851 and Cannell et al. in U.S. Pat. No. 6,013,250, all of which are incorporated herein by reference. For example, hair care compositions can be aqueous, alcoholic or aqueous-alcoholic solutions, the alcohol preferably being ethanol or isopropanol, in a proportion of from about 1 to about 75% by weight relative to the total weight, for the aqueous-alcoholic solutions. Additionally, the hair care compositions may contain one or more conventional cosmetic or dermatological additives or adjuvants including but not limited to, antioxidants, preserving agents, fillers, surfactants, UVA and/or UVB sunscreens, fragrances, thickeners, gelling agents, wetting agents and anionic, nonionic or amphoteric polymers, and dyes or pigments.

The hair care compositions and methods may also include at least one coloring agents such as any dye, lake, pigment, and the like that may be used to change the color of hair, skin, or nails. Hair coloring agents are well known in the art (see for example Green et al. supra, CFTA International Color Handbook, 2^{nd} ed., Micelle Press, England (1992) and Cosmetic Handbook, US Food and Drug Administration, FDA/IAS Booklet (1992)), and are available commercially from various sources (for example Bayer, Pittsburgh, Pa.; Ciba-Geigy, Tarrytown, N.Y.; ICI, Bridgewater, N.J.; Sandoz, Vienna, Austria; BASF, Mount Olive, N.J.; and Hoechst, Frankfurt, Germany). Suitable hair coloring agents include, but are not limited to dyes, such as 4-hydroxypropylamino-3-nitrophenol, 4-amino-3-nitrophenol, 2-amino-6-chloro-4-nitrophenol, 2-nitro-paraphenylenediamine, N,N-hydroxyethyl-2-nitro-phenylenediamine, 4-nitro-indole, Henna, HC Blue 1, HC Blue 2, HC Yellow 4, HC Red 3, HC Red 5, Disperse Violet 4, Disperse Black 9, HC Blue 7, HC Blue 12, HC Yellow 2, HC Yellow 6, HC Yellow 8, HC Yellow 12, HC Brown 2, D&C Yellow 1, D&C Yellow 3, D&C Blue 1, Disperse Blue 3, Disperse violet 1, eosin derivatives such as D&C Red No. 21 and halogenated fluorescein derivatives such as D&C Red No. 27, D&C Red Orange No. 5 in combination with D&C Red No. 21 and D&C Orange No. 10; and pigments, such as D&C Red No. 36 and D&C Orange No. 17, the calcium lakes of D&C Red Nos. 7, 11, 31 and 34, the barium lake of D&C Red No. 12, the strontium lake of D&C Red No. 13, the aluminum lakes of FD&C Yellow No. 5, of FD&C Yellow No. 6, of D&C Red No. 27, of D&C Red No. 21, and of FD&C Blue No. 1, iron oxides, manganese violet, chromium oxide, titanium dioxide, titanium dioxide nanoparticles, zinc oxide, barium oxide, ultramarine blue, bismuth citrate, and carbon black particles. In one embodiment, the hair coloring agents are D&C Yellow 1 and 3, HC Yellow 6 and 8, D&C Blue 1, HC Blue 1, HC Brown 2, HC Red 5,2-nitro-paraphenylenediamine, N,N-hydroxyethyl-2-nitro-phenylenediamine, 4-nitro-indole, and carbon black. Metallic and semiconductor nanoparticles may also be used as hair coloring agents due to their strong emission of light (U.S. Patent Application Publication No. 2004-0010864 to Vic et al.).

Hair care compositions may include, but not limited to shampoos, conditioners, lotions, aerosols, gels, mousses, and hair dyes.

In one embodiment, a hair care product is provided comprising:

a) a non-aqueous composition comprising a mixture of:
1) at least one substrate selected from the group consisting of:
   i) esters having the structure

[X]ₘR₅
wherein X=an ester group of the formula R₆C(O)O
R₆=C1 to C7 linear, branched or cyclic hydrocarbyl moiety, optionally substituted with hydroxyl groups or C1 to C4 alkoxy groups, wherein R₆ optionally comprises one or more ether linkages for R₆=C2 to C7;
R₅=a C1 to C6 linear, branched, or cyclic hydrocarbyl moiety or a five-membered cyclic heteroaromatic moiety or six-membered cyclic aromatic or heteroaromatic moiety optionally substituted with hydroxyl groups;
wherein each carbon atom in R₅ individually comprises no more than one hydroxyl group or no more than one ester group or carboxylic acid group;
wherein R₅ optionally comprises one or more ether linkages;
m is an integer ranging from 1 to the number of carbon atoms in R₅; and
wherein said esters have a solubility in water of at least 5 ppm at 25° C.;
   ii) glycerides having the structure

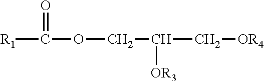

wherein R₁=C1 to C7 straight chain or branched chain alkyl optionally substituted with an hydroxyl or a C1 to C4 alkoxy group and R₃ and R₄ are individually H or R₁C(O);
iii) one or more esters of the formula

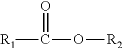

wherein R₁ is a C1 to C7 straight chain or branched chain alkyl optionally substituted with an hydroxyl or a C1 to C4 alkoxy group and R₂ is a C1 to C10 straight chain or branched chain alkyl, alkenyl, alkynyl, aryl, alkylaryl, alkylheteroaryl, heteroaryl, (CH₂CH₂O)ₙ, or (CH₂CH(CH₃)—O)ₙH and n is 1 to 10; and
iv) acetylated saccharides selected from the group consisting of acetylated monosaccharides, acetylated disaccharides, and acetylated polysaccharides; and
2) a solid source of peroxygen comprising a perborate, a percarbonate or a combination thereof;
3) an optional organic cosolvent; and

b) an aqueous composition comprising
1) an enzyme catalyst having perhydrolytic activity; and
2) at least one buffer; wherein the aqueous composition comprises a pH of at least 4; and
wherein the non-aqueous composition and the aqueous compositions remain separated prior to use and wherein an enzymatically generated peracid is produced upon combining the non-aqueous and aqueous compositions.

The buffer(s) in the aqueous composition should be capable of maintaining the aqueous solution during storage at a pH of at least 4. In a preferred aspect, the aqueous composition components are selected to maintain a pH of at least about 4 to about 9. The resulting pH obtained upon combining the reaction components should be in a range where the enzyme catalyst has perhydrolytic activity and is capable of catalyzing the production of at least one peracid.

In one embodiment, the optional organic cosolvent is propylene glycol, dipropylene glycol, triethylene glycol, 1,3-propanediol, 1,3-butanediol, hexylene glycol, or any combination thereof.

In one embodiment, the buffer is selected from the group consisting of acetate, citrate, phosphate, pyrophosphate, glycine, bicarbonate, methylphosphonate, succinate, malate, fumarate, tartrate, maleate, and combinations thereof.

In another embodiment, the enzyme catalyst having perhydrolytic activity is in the form of a fusion protein comprising:

a) a first portion comprising the enzyme having perhydrolytic activity; and

b) a second portion having a peptidic component having affinity for human hair.

In a further aspect, the fusion protein has the following general structure:

PAH-[L]_{y}-HSBD

or

HSBD-[L]_{y}-PAH
wherein
PAH is the enzyme having perhydrolytic activity;
HSBD is a peptidic component having affinity for hair;
L is a linker ranging from 1 to 100 amino acids in length; and
y is 0 or 1.

The non-aqueous composition and the aqueous composition of the above hair care product remain separated until use. As such, the hair care product is in the form of a multi-compartment packet, a multi-compartment bottle, at least two individual containers, and combinations thereof.

The non-aqueous component is substantially free of water until use (i.e. until the reaction components are combined to initiate enzymatic perhydrolysis). In one embodiment, the non-aqueous component may further comprise at least one desiccant.

In one embodiment, a hair care composition is provided comprising:

a) an enzyme catalyst having perhydrolytic activity, wherein said enzyme catalyst comprises an enzyme having a CE-7 signature motif that aligns with a reference sequence SEQ ID NO: 2 using CLUSTALW, said signature motif comprising:
i) an RGQ motif at positions corresponding to positions 118-120 of SEQ ID NO:2;
ii) a GXSQG motif at positions corresponding to positions 179-183 of SEQ ID NO:2; and
iii) an HE motif at positions corresponding to positions 298-299 of SEQ ID NO:2; and

b) at least one substrate selected from the group consisting of:
i) esters having the structure

[X]ₘR₅
wherein X=an ester group of the formula R₆C(O)O
R₆=C1 to C7 linear, branched or cyclic hydrocarbyl moiety, optionally substituted with hydroxyl groups or C1 to C4 alkoxy groups, wherein R₆ optionally comprises one or more ether linkages for R₆=C2 to C7;
R₅=a C1 to C6 linear, branched, or cyclic hydrocarbyl moiety or a five-membered cyclic heteroaromatic moiety or six-membered cyclic aromatic or heteroaromatic moiety optionally substituted with hydroxyl groups; wherein each carbon atom in R5 individually comprises no more than one hydroxyl group or no more than one ester group or carboxylic acid group; wherein R₅ optionally comprises one or more ether linkages;
m is an integer ranging from 1 to the number of carbon atoms in R₅; and
wherein said esters have a solubility in water of at least 5 ppm at 25° C.;
ii) glycerides having the structure

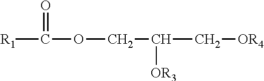

wherein R₁=C1 to C7 straight chain or branched chain alkyl optionally substituted with an hydroxyl or a C1 to C4 alkoxy group and R₃ and R₄ are individually H or R₁C(O);
iii) one or more esters of the formula

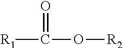

wherein R₁ is a C1 to C7 straight chain or branched chain alkyl optionally substituted with an hydroxyl or a C1 to C4 alkoxy group and R₂ is a C1 to C10 straight chain or branched chain alkyl, alkenyl, alkynyl, aryl, alkylaryl, alkylheteroaryl, heteroaryl, (CH₂CH₂O)ₙ, or (CH₂CH(CH₃)—O)ₙH and n is 1 to 10; and
iv) acetylated saccharides selected from the group consisting of acetylated monosaccharides, acetylated disaccharides, and acetylated polysaccharides;

c) a source of peroxygen; and

d) a dermally acceptable carrier medium; wherein the composition comprises peracid when (a), (b), and (c) are combined.

In another embodiment, the perhydrolytic enzyme used in the hair care composition is a fusion protein comprising

a) a first portion comprising the enzyme having perhydrolytic activity and

b) a second portion having affinity for hair.

In one embodiment, the peracid formed in the hair care composition is peracetic acid.

The components of the hair care composition may remain separated until use. In one embodiment, the peracid-generating components are combined and then contacted with the hair surface whereby the resulting peracid-based benefit agent provides a benefit selected from the group consisting of hair removal, hair weakening (as measured by a decrease in the tensile strength of hair), hair bleaching, hair dye pretreating (oxidative hair dyes), hair curling, and hair conditioning (i.e., a one-step application method). In another embodiment, the peracid-generating components are combined such that the peracid is produced in situ. The relative amount of the ingredients in the hair care composition may be varied according to the desired effect.

In one embodiment a single-step hair treatment method is provided comprising:

1) providing a set of reaction components comprising:
a) at least one substrate selected from the group consisting of:
   i) esters having the structure

[X]ₘR₅
wherein X=an ester group of the formula R₆C(O)O
R₆=C1 to C7 linear, branched or cyclic hydrocarbyl moiety, optionally substituted with hydroxyl groups or C1 to C4 alkoxy groups, wherein R₆ optionally comprises one or more ether linkages for R₆=C2 to C7;
R₅=a C1 to C6 linear, branched, or cyclic hydrocarbyl moiety or a five-membered cyclic heteroaromatic moiety or six-membered cyclic aromatic or heteroaromatic moiety optionally substituted with hydroxyl groups; wherein each carbon atom in R₅ individually comprises no more than one hydroxyl group or no more than one ester group or carboxylic acid group; wherein R₅ optionally comprises one or more ether linkages;
m is an integer ranging from 1 to the number of carbon atoms in R₅; and
wherein said esters have a solubility in water of at least 5 ppm at 25° C.;
ii) glycerides having the structure

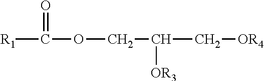

wherein R₁=C1 to C7 straight chain or branched chain alkyl optionally substituted with an hydroxyl or a C1 to C4 alkoxy group and R₃ and R₄ are individually H or R₁C(O);
iii) one or more esters of the formula

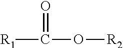

wherein R₁ is a C1 to C7 straight chain or branched chain alkyl optionally substituted with an hydroxyl or a C1 to C4 alkoxy group and R₂ is a C1 to C10 straight chain or branched chain alkyl, alkenyl, alkynyl, aryl, alkylaryl, alkylheteroaryl, heteroaryl, (CH₂CH₂O)ₙ, or (CH₂CH(CH₃)—O)ₙH and n is 1 to 10; and
iv) acetylated saccharides selected from the group consisting of acetylated monosaccharides, acetylated disaccharides, and acetylated polysaccharides;
b) a source of peroxygen; and
c) an enzyme catalyst having perhydrolytic activity, wherein said enzyme catalyst comprises an enzyme having a CE-7 signature motif that aligns with a reference sequence SEQ ID NO: 2 using CLUSTALW, said signature motif comprising:
   i) an RGQ motif at positions corresponding to positions 118-120 of SEQ ID NO:2;
   ii) a GXSQG motif at positions corresponding to positions 179-183 of SEQ ID NO:2; and
   iii) an HE motif at positions corresponding to positions 298-299 of SEQ ID NO:2; and

2) combining the reaction components of (1), whereby at least one peracid is produced; and

3) contacting hair with said peracid; whereby the resulting peracid-based benefit agent provides a benefit selected from the group consisting of hair removal, hair weakening, hair bleaching, hair dye pretreating, hair curling, and hair conditioning; wherein one or more components of a cosmetically acceptable media may be present.

One or two of the individual components of the peracid generating system (i.e., sequential application on the hair surface) composition may be contacted with the hair surface prior to applying the remaining components required for enzymatic peracid production. In one embodiment, the perhydrolytic enzyme is contacted with the hair prior to the substrate and the source of peroxygen (i.e., a “two-step application”). In a preferred embodiment, the enzyme having perhydrolytic activity is a targeted perhydrolase (i.e., fusion protein) that is applied to the hair surface prior to the remaining components necessary for enzymatic peracid production (i.e., a two-step application method).

In another embodiment, a method is provided comprising

1) contacting hair with a fusion protein comprising;
a) a first portion comprising an enzyme having perhydrolytic activity, wherein said enzyme having a CE-7 signature motif that aligns with a reference sequence SEQ ID NO: 2 using CLUSTALW, said signature motif comprising:
   i) an RGQ motif at positions corresponding to positions 118-120 of SEQ ID NO:2;
   ii) a GXSQG motif at positions corresponding to positions 179-183 of SEQ ID NO:2; and
   iii) an HE motif at positions corresponding to positions 298-299 of SEQ ID NO:2; and
b) a second portion comprising a peptidic component having affinity for hair; whereby the fusion peptide binds to the hair;

2) optionally rinsing the hair with an aqueous solution to remove unbound fusion peptide;

3) contacting the hair comprising bound fusion peptide with
a) at least one substrate selected from the group consisting of:
   i) esters having the structure

[X]ₘR₅
wherein X=an ester group of the formula R₆C(O)O
R₆=C1 to C7 linear, branched or cyclic hydrocarbyl moiety, optionally substituted with hydroxyl groups or C1 to C4 alkoxy groups, wherein R₆ optionally comprises one or more ether linkages for R₆=C2 to C7;
R₅=a C1 to C6 linear, branched, or cyclic hydrocarbyl moiety or a five-membered cyclic heteroaromatic moiety or six-membered cyclic aromatic or heteroaromatic moiety optionally substituted with hydroxyl groups; wherein each carbon atom in R₅ individually comprises no more than one hydroxyl group or no more than one ester group or carboxylic acid group; wherein R₅ optionally comprises one or more ether linkages;
m is an integer ranging from 1 to the number of carbon atoms in R₅; and
wherein said esters have a solubility in water of at least 5 ppm at 25° C.;
ii) glycerides having the structure

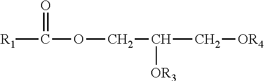

wherein R₁=C1 to C7 straight chain or branched chain alkyl optionally substituted with an hydroxyl or a C1 to C4 alkoxy group and R₃ and R₄ are individually H or R₁C(O);
iii) one or more esters of the formula

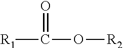

wherein R₁ is a C1 to C7 straight chain or branched chain alkyl optionally substituted with an hydroxyl or a C1 to C4 alkoxy group and R₂ is a C1 to C10 straight chain or branched chain alkyl, alkenyl, alkynyl, aryl, alkylaryl, alkylheteroaryl, heteroaryl, (CH₂CH₂O)ₙ, or (CH₂CH(CH₃)—O)ₙH and n is 1 to 10; and
iii) acetylated saccharides selected from the group consisting of acetylated monosaccharides, acetylated disaccharides, and acetylated polysaccharides; and
b) a source of peroxygen; whereby upon combining the fusion peptide with the substrate and the source of peroxygen a peracid is produced; whereby the resulting peracid provides a benefit selected from the group consisting of hair removal, hair weakening, hair bleaching, hair dye pretreating, hair curling, and hair conditioning.

In a preferred embodiment, the above peracid-based hair care methods is used to remove hair and/or weaken the tensile strength of hair. The hair care methods direct to hair removal or tensile strength reduction may optionally include a reducing agent, such as a thioglycolate, to enhance the weakening and/or removal of the hair from the surface comprising the hair targeted for removal.

In a further embodiment, the above hair depilatory methods may be used as a pre-treatment for subsequence application of a commercial hair removal product comprising at least one reducing agent, such as a thioglycolate-based hair removal product. As such, the above method may include the step of contacting the peracid treated hair with a reducing agent. Preferably the reducing agent is a thioglycolate, such as sodium thioglycolate or potassium thioglycolate (e.g., an active ingredient often used in hair removal products such as NAIR®).

### Recombinant Microbial Expression

The genes and gene products of the instant sequences may be produced in heterologous host cells, particularly in the cells of microbial hosts. Preferred heterologous host cells for expression of the instant genes and nucleic acid molecules are microbial hosts that can be found within the fungal or bacterial families and which grow over a wide range of temperature, pH values, and solvent tolerances. For example, it is contemplated that any of bacteria, yeast, and filamentous fungi may suitably host the expression of the present nucleic acid molecules. The perhydrolase may be expressed intracellularly, extracellularly, or a combination of both intracellularly and extracellularly, where extracellular expression renders recovery of the desired protein from a fermentation product more facile than methods for recovery of protein produced by intracellular expression. Transcription, translation and the protein biosynthetic apparatus remain invariant relative to the cellular feedstock used to generate cellular biomass; functional genes will be expressed regardless. Examples of host strains include, but are not limited to, bacterial, fungal or yeast species such as Aspergillus, Trichoderma, Saccharomyces, Pichia, Phaffia, Kluyveromyces, Candida, Hansenula, Yarrowia, Salmonella, Bacillus, Acinetobacter, Zymomonas, Agrobacterium, Etythrobacter, Chlorobium, Chromatium, Flavobacterium, Cytophaga, Rhodobacter, Rhodococcus, Streptomyces, Brevibacterium, Corynebacteria, Mycobacterium, Deinococcus, Escherichia, Erwinia, Pantoea, Pseudomonas, Sphingomonas, Methylomonas, Methylobacter, Methylococcus, Methylosinus, Methylomicrobium, Methylocystis, Alcaligenes, Synechocystis, Synechococcus, Anabaena, Thiobacillus, Methanobacterium, Klebsiella, and Myxococcus. In one embodiment, bacterial host strains include Escherichia, Bacillus, Kluyveromyces, and Pseudomonas. In a preferred embodiment, the bacterial host cell is Bacillus subtilis or Escherichia coli.

Large-scale microbial growth and functional gene expression may use a wide range of simple or complex carbohydrates, organic acids and alcohols or saturated hydrocarbons, such as methane or carbon dioxide in the case of photosynthetic or chemoautotrophic hosts, the form and amount of nitrogen, phosphorous, sulfur, oxygen, carbon or any trace micronutrient including small inorganic ions. The regulation of growth rate may be affected by the addition, or not, of specific regulatory molecules to the culture and which are not typically considered nutrient or energy sources.

Vectors or cassettes useful for the transformation of suitable host cells are well known in the art. Typically the vector or cassette contains sequences directing transcription and translation of the relevant gene, a selectable marker, and sequences allowing autonomous replication or chromosomal integration. Suitable vectors comprise a region 5′ of the gene which harbors transcriptional initiation controls and a region 3′ of the DNA fragment which controls transcriptional termination. It is most preferred when both control regions are derived from genes homologous to the transformed host cell and/or native to the production host, although such control regions need not be so derived.

Initiation control regions or promoters which are useful to drive expression of the present cephalosporin C deacetylase coding region in the desired host cell are numerous and familiar to those skilled in the art. Virtually any promoter capable of driving these genes is suitable for the present invention including but not limited to, CYC1, HIS3, GAL1, GAL10, ADH1, PGK, PHO5, GAPDH, ADC1, TRP1, URA3, LEU2, ENO, TPI (useful for expression in Saccharomyces); AOX1 (useful for expression in Pichia); and lac, araB, tet, trp, IP_{L}, IP_{R}, T7, tac, and trc (useful for expression in Escherichia coli) as well as the amy, apr, npr promoters and various phage promoters useful for expression in Bacillus.

Termination control regions may also be derived from various genes native to the preferred host cell. In one embodiment, the inclusion of a termination control region is optional. In another embodiment, the chimeric gene includes a termination control region derived from the preferred host cell.

### Industrial Production

A variety of culture methodologies may be applied to produce the perhydrolase catalyst. For example, large-scale production of a specific gene product over-expressed from a recombinant microbial host may be produced by batch, fed-batch, and continuous culture methodologies. Batch and fed-batch culturing methods are common and well known in the art and examples may be found in Thomas D. Brock in Biotechnology: A Textbook of Industrial Microbiology, Second Edition, Sinauer Associates, Inc., Sunderland, Mass. (1989) and Deshpande, Mukund V., Appl. Biochem. Biotechnol., 36:227-234 (1992).

Commercial production of the desired perhydrolase catalyst may also be accomplished with a continuous culture. Continuous cultures are an open system where a defined culture media is added continuously to a bioreactor and an equal amount of conditioned media is removed simultaneously for processing. Continuous cultures generally maintain the cells at a constant high liquid phase density where cells are primarily in log phase growth. Alternatively, continuous culture may be practiced with immobilized cells where carbon and nutrients are continuously added, and valuable products, by-products or waste products are continuously removed from the cell mass. Cell immobilization may be performed using a wide range of solid supports composed of natural and/or synthetic materials.

Recovery of the desired perhydrolase catalysts from a batch fermentation, fed-batch fermentation, or continuous culture, may be accomplished by any of the methods that are known to those skilled in the art. For example, when the enzyme catalyst is produced intracellularly, the cell paste is separated from the culture medium by centrifugation or membrane filtration, optionally washed with water or an aqueous buffer at a desired pH, then a suspension of the cell paste in an aqueous buffer at a desired pH is homogenized to produce a cell extract containing the desired enzyme catalyst. The cell extract may optionally be filtered through an appropriate filter aid such as celite or silica to remove cell debris prior to a heat-treatment step to precipitate undesired protein from the enzyme catalyst solution. The solution containing the desired enzyme catalyst may then be separated from the precipitated cell debris and protein by membrane filtration or centrifugation, and the resulting partially-purified enzyme catalyst solution concentrated by additional membrane filtration, then optionally mixed with an appropriate carrier (for example, maltodextrin, phosphate buffer, citrate buffer, or mixtures thereof) and spray-dried to produce a solid powder comprising the desired enzyme catalyst.

When an amount, concentration, or other value or parameter is given either as a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope be limited to the specific values recited when defining a range.

### General Methods

The following examples are provided to demonstrate preferred aspects of the invention. It should be appreciated by those of skill in the art that the techniques disclosed in the examples follow techniques to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the presently disclosed methods and examples.

All reagents and materials were obtained from DIFCO Laboratories (Detroit, Mich.), GIBCO/BRL (Gaithersburg, Md.), TCI America (Portland, Oreg.), Roche Diagnostics Corporation (Indianapolis, Ind.) or Sigma/Aldrich Chemical Company (St. Louis, Mo.), unless otherwise specified.

The following abbreviations in the specification correspond to units of measure, techniques, properties, or compounds as follows: “sec” or “s” means second(s), “min” means minute(s), “h” or “hr” means hour(s), “μL” means microliter(s), “mL” means milliliter(s), “L” means liter(s), “mM” means millimolar, “M” means molar, “mmol” means millimole(s), “ppm” means part(s) per million, “wt” means weight, “wt %” means weight percent, “g” means gram(s), “mg” means milligram(s), “μg” means microgram(s), “ng” means nanogram(s), “g” means gravity, “gf” means maximum grams force, “den” means denier, “N” means Newtons, “tex” means basic tex unit in mass of yard/fiber in grams per 1000 meters length, “HPLC” means high performance liquid chromatography, “dd H₂O” means distilled and deionized water, “dcw” means dry cell weight, “ATCC” or “ATCC®” means the American Type Culture Collection (Manassas, Va.), “U” means unit(s) of perhydrolase activity, “rpm” means revolution(s) per minute, “Tg” means glass transition temperature, “Ten.” means tenacity, “TS” means tensile strength, and “EDTA” means ethylenediaminetetraacetic acid.

Expression Vector pLD001

Plasmid pLD001 (SEQ ID NO: 292) has been previous reported as a suitable expression vector for E. coli (see U.S. Patent Application Publication No. 2010-0158823 A1 to Wang et al.; incorporated herein by reference).

The vector pLD001 was derived from the commercially available vector pDEST17 (Invitrogen, Carlsbad, Calif.). It includes sequences derived from the commercially available vector pET31 b (Novagen, Madison, Wis.) that encode a fragment of the enzyme ketosteroid isomerase (KSI). The KSI fragment was included as a fusion partner to promote partition of the peptides into insoluble inclusion bodies in E. coli. The KSI-encoding sequence from pET31b was modified using standard mutagenesis procedures (QuickChange II, Stratagene, La Jolla, Calif.) to include three additional Cys codons, in addition to the one Cys codon found in the wild type KSI sequence. In addition, all Asp codons in the coding sequence were replaced by Glu codons. The plasmid pLD001, given by SEQ ID NO: 292, was constructed using standard recombinant DNA methods, which are well known to those skilled in the art.

Coding sequences bounded by BamHI and AscI sites may be ligated between BamHI and AscI sites in pLD001 using standard recombinant DNA methods. The resulting gene fusions resulted in a peptide of interest was fused downstream from a modified fragment of ketosteroid isomerase (KSI(C4)E) that served to drive the peptide into insoluble inclusion bodies in E. coli (See U.S. Patent Application Publication No. 2009-0029420A1; herein incorporated by reference).

### Construction of Hair-Targeted Perhydrolase Fusions

The following describes the design of an expression system for the production of perhydrolases targeted to hair via hair-binding sequences.

The genes (SEQ ID NO: 286 and SEQ ID NO: 287) encoding for fusions of an enzyme having perhydrolytic activity (a “perhydrolase”) to hair-binding domains (SEQ ID NO: 290 and SEQ ID NO: 291) were designed to have the polynucleotide sequence of the C277S variant of the Thermotoga maritime perhydrolase (SEQ ID NO: 293) fused at the 3′-end to the nucleotide sequence encoding a flexible linker; itself further fused to the hair-binding domains HC263 or HC1010 (SEQ ID NO: 290 and SEQ ID NO: 291; respectively). The genes were codon-optimized for expression in E. coli and synthesized by DNA2.0 (Menlo Park, Calif.). The genes were cloned behind the T7 promoter in the expression vector pLD001 (SEQ ID NO: 292) between the NdeI and AscI restriction sites yielding plasmids pLR1021 and pLR1022, respectively. To express the fusion protein, the plasmids were transferred to the E. coli strain BL21Al (Invitrogen, Carlsbad, Calif.) yielding strains LR3311 (perhydrolase fusion to HC263; SEQ ID NO: 288) and LR3312 (perhydrolase fusion to HC1010; SEQ ID NO: 289).

The non-targeted C277S variant of the Thermotoga maritime perhydrolase was cloned similarly. The preparation and recombinant expression of the Thermotoga maritime C277S variant has previously been reported by DiCosimo et al. in U.S. Patent Application Publication No. 2010-0087529; hereby incorporated by reference.

### HPLC Karst Assay Procedure

The following assay procedure was adapted from the procedure reported by U. Karst et al. Anal. Chem. 1997, 69(17):3623-3627.

Assay Procedure
1. Add 300 μL at dd H₂O (400 μL for blank with no sample) to a 2.0-mL HPLC screw cap vial (Agilent-5182-0715). Prepare one vial for each sample.
2. Add 100 μL of 20 mM MTS (Methyl p-tolyl sulfide; Aldrich 7596-25g; fw 138.23; 99% pure)/acetonitrile solution using a 250-μL gas-tight syringe to each vial and swirl to mix.
3. Add 100 μL of the H3PO4 diluted and quenched sample to each vial and swirl to mix.
4. Place vials in a light-proof box and allow assay reaction to proceed in the dark for 10 min with no stirring.
5. Remove vials from light-proof box, add 400 μL acetonitrile to each vial, and swirl to mix.
6. Add 100 μL of 120 mM TPP (triphenyl phosphine, Aldrich T84409-25g; FW 262.29; 99% pure)/acetonitrile solution using a 250-μL gas-tight syringe to each vial, cap vial (Agilent-5182-0723). Vortex to mix.
7. Place vials in the light-proof box and allow the assay to continue in the dark for 30 min with no stirring.
8. Remove vials from light-proof box, add 100 μL of 2.5 mM DEET (N,N-diethyl-m-toluamide, Aldrich-D100951-100g; FW-191.27; 97% pure)/acetonitrile solution (used as HPLC external standard) using a 250-μL gas-tight syringe to each vial and immediately inject on HPLC for analysis. (Total volume of assay solution is 1100 μL)

### HPLC Analysis

The following HPLC conditions were used: Supelco Discovery C8 column (15 cm×4.0 mm, 5 um; Supelco #59353-U40) with Supelguard Discovery C8 Supelguard cartridges.

Mobile phase: 41-100% acetonitrile/59-0% distilled water, 1 mL/min gradient. Injection volume, 15 μL; analysis time, 10 min. Detector—UV absorbance at 225 nm.
Gradient program using CH₃CN (Sigma-34851-1 L) and dd H₂O:

| Time (min:sec) | (% CH₃CN) | (% ddH₂O) |
|---|---|---|
| 0:00 | 41 | 59 |
| 3:00 | 41 | 59 |
| 3:10 | 100 | 0 |
| 6.0 | 100 | 0 |
| 6.1 | 41 | 59 |
| 10.0 | 41 | 59 |

### Hair Tress Tensile Strength Testing Procedure

This tensile strength test procedure was developed for hair bundles containing multiple hair fibers and the results would reflect treatment effects averaged over multiple hair fibers. The hair samples were cut into 4 cm long, 2 mm wide hair bundle of approximately 30-70 mg hair, held together by a 1 mm thick, and 5 mm long glue strip. 5 mm of the free end of this tress was further glued using a fast drying glue (such as DUCO® CEMENT®, a nitro cellulose household cement). After drying the glue, any loose hair strands were cut off and the sample was weighed.

COM-TEN® Tensile Tester 95-VD (Corn-Ten Industries, Pinellas Park, Fla.), fitted with a 100 lb load-cell was used for tensile tests. In order to reduce sample slippage, 5 mm wide strips of industrial grade VELCRO® (Velcro USA, Manchester, N.H.) were attached to the inside edges of the clamps. Before testing the CALIBRATION was set to “off”, FORCE UNITS were set to “grams” and the distance between the clamps was adjusted to 3 cm. The test sample was soaked in water for 30 seconds. Excess moisture was removed by gentle absorption on a paper towel, leaving enough moisture in hair for it to qualify as being at 100% humidity level. The glued edges of the test sample were clamped at both upper and lower clamps in such a way that the VELCRO® strips held the hair just below the glue. Tester speed was set to ˜2.5 inches by adjusting the speed control knob. With the Force meter in RUN mode, TARE was set to ZERO to set the starting PEAK FORCE to 0. To start the test the DIRECTION toggle switch was pressed to UP position. At the conclusion of the test, when the sample failed, the DIRECTION switch was moved to STOP and the peak force was recorded. The hair was cut off along the edge of the clamps at both lower and upper clamps. The clamps were opened and the stubs were removed, dried in air and weighed. The difference in original sample weight and combined weights of the stubs was the weight of the hair undergoing tensile elongation, and this quantity was used to calculate the tensile strength.

For the purpose of comparisons of samples following the treatments, the tensile strengths were defined as follows:

Tensile Strength (N/mg hair)=Peak force (Newtons)/(Initial sample weight−weight of stubs)

Benchmarking the assay was achieved by measuring the tensile-strength (Hair-weakening) of hair-tresses after treatment with a commercially available depilatory product, NAIR® Lotion with Cocoa Butter (Church & Dwight Co., Inc., Princeton, N.J.). Based on the NAIR® product instruction, the recommended treatment time is 5-10 min. Therefore, the tensile strength of a hair sample treated with NAIR® between 5 min to 10 min was used to determine the target level. Test hair sample consisted of a hair bundle of approximately 50 mg hair of 4 cm length, held together by a 1 mm thick, 2 mm wide and 5 mm long glue strip. The test-sample was placed on a glass plate. Approximately 1 mL of NAIR® lotion was applied to the tress with a gloved finger. The lotion was gently spread over and pressed into the tress to cover all hair fibers. After the desired treatment time at room temperature, the tress was rinsed thoroughly with tap water to remove all traces of the lotion. The sample was air-dried and tested for its tensile strength.

For these treatment times, the tensile strengths of the tresses (wet tress, 100% humidity) were found to be between ˜0.2 N/mg hair for 10 min and between 0.7-1.4 N/mg hair for 5 min. The data is provided in Table 1. Given the variation in the tensile strength the desired level of hair weakening efficacy was targeted for 1.5 N/mgH as NAIR® 5 min treatment benchmark.

TABLE 1
Result of benchmarking tensile assay.

| | | | | Treatment | TS, |
|---|---|---|---|---|---|
| Experiment | Sample | Hair state | Humidity | time, min | N/mgH** |
| 1 | 1 | wet | 100% | 5 | 0.74 |
| 2 | 2 | wet | 100% | 5 | 1.00 |
| 3 | 3 | wet | 100% | 5 | 1.18 |
| 4 | 4 | wet | 100% | 5 | 1.42 |
| 5 | 5 | dry | 10-20% | 5 | 2.53 |
| 6 | 6 | wet | 100% | 10 | 0.17 |
| 7 | 7 | wet | 100% | 10 | 0.18 |
| 8 | 8 | wet | 100% | 10 | 0.18 |
| 9 | 8 | wet | 100% | 10 | 0.24 |
| 10 | 10 | dry | 10-20% | 10 | 1.15 |

**TS is average (of 2 samples) tensile strength, expressed as Newton per milligram hair (N/mgH)

### Hair Color Measurement Procedure

Hair tresses were dried under air and color measurements were made using X-RITE® SP64 spectrophotometer (X-Rite, Grandville, Mich.) with 4 mm port. Color numbers were measured at D65/10° from reflectance, according to CIELAB76. Hair tresses (all replicates) were placed under a card paper with punched out holes, making sure that the background was not visible. The port-hole of the spectrophotometer was centered on the hole to scan the hair sample underneath. The tress-bundle was turned over and placed under the card and an additional measurement was made. Average L*, a*, b* (color according to CIELAB76) values were recorded.

ΔE of color loss was calculated according to the following formula:

ΔE=((L*−L*_{ref})²+(a*−a*_{ref})²+(b*−b*_{ref})²)^{0.5}

### Where,

L*, a* and b* are L*, a* and b* values for a sample tress after treatment,
L_{ref}*, a_{ref}* and b_{ref}* are L*, a* and b* values for untreated hair

### Example 1

### Production of the Fusion Proteins

This example describes the expression and purification of perhydrolases targeted to hair via hair-binding domains.

Strains LR3311 and strain LR3312 were grown in 1 liter of autoinduction medium (10 g/L tryptone, 5 g/L yeast extract, 5 g/L NaCl, 50 mM Na₂HPO₄, 50 mM KH₂PO₄, 25 mM (NH₄)₂SO₄, 3 mM MgSO₄, 0.75% glycerol, 0.075% glucose and 0.05% arabinose) containing 50 mg/L spectinomycin at 37° C. for 20 hrs under 200 rpm agitation. Production of the untargeted perhydrolase has been described previously in U.S. Patent Application Publication No. 2010-0087529 to DiCosimo et al.

The cells were harvested by centrifugation at 8000 rpm at 4° C. and washed by resuspending the cell pellets in 300 mL of ice chilled lysis buffer (50 mM Tris pH 7.5, 5 mM EDTA, 100 mM NaCl) using a tissue homogenizer (Brinkman Homogenizer model PCU11; Brinkmann Instruments, Mississauga, Canada) at 3500 rpm followed by centrifugation (8000 rpm, 4° C.). The cells were then lysed by resuspension in chilled lysis buffer containing 75 mg of chicken egg white lysozyme (Sigma) using the tissue homogenizer. The cell suspensions were allowed to rest on ice for 3 hrs to allow the digestion of the cell wall by the lysozyme, with periodic homogenization with the tissue homogenizer. At this stage, care was taken to avoid any foaming of the extracts. The extracts were split (150 mL per 500-mL bottle) and frozen at −20° C. The frozen cell extracts were thawed at room temperature (˜22° C.), homogenized with the tissue homogenizer and disrupted by sonication using a sonicator (Branson Ultrasonics Corporation, Danbury, Conn.; Sonifier model 450) equipped with a 5 mm probe at 20% maximum output, 2 pulses per second for 1 min. The lysed cell extracts were transferred to 4×50-mL conical polypropylene centrifuge tubes and then centrifuged at 10,000 rpm for 10 min at 4° C. The pellet containing cell debris as well as unbroken cells was frozen. Aliquots of the lysate were transferred to 15-mL conical polypropylene tube (12×5-mL) and heated to 80° C. for 15 min, chilled on ice, and pooled into 4×50-mL conical polypropylene centrifuge tubes. The soluble fraction containing the thermostable enzyme and the precipitated E. coli proteins were separated by centrifugation at 10,000 rpm for 10 min at 4° C. If the cell disruption was incomplete after the sonication step, the frozen pellet was thawed again and subjected to a second round of sonication, centrifugation and heat treatment. The output of this purification protocol typically yielded 2-4 mg of protein per mL with a purity of the fusion perhydrolase between 90% and 75% of the protein as estimated by polyacrylamide gel electrophoresis (PAGE) analysis. Total protein was quantitated by the bicinchoninic acid (BCA) assay (Thermo Fisher Scientific, Rockford, Ill.) using a solution of Bovine Serum Albumin as a standard.

### Example 2

### Binding of the Hair-Targeted Perhydrolase Fusion to Hair

This example demonstrates the binding of the perhydrolase to hair in a manner dependent on the fusion of hair-binding sequences to the perhydrolase.

For hair binding experiments brown hair tresses (International Hair Importers and Products, Glensdale N.Y.) were used. The hair was washed with 2% SLES, rinsed extensively with deionized water and air dried.

Around 20 mg of 1 cm brown hair fragments was added in a 1.8-mL microfuge tube. Hydrolase assay buffer (1.2 mL) as added to the hair followed by the addition of the perhydrolase enzymes to the solution. The enzymes were allowed to bind the hair for 30 min with gentle agitation (24 rpm) on an Adams Nutator (model 1105, Becton Dickinson, Franklin Lakes, N.J.). No enzyme controls, with hair and without hair, were included in the binding experiment to account for non-enzymatic hydrolysis of the pNPA hydrolase reagent. After the binding step, a 1.0-mL aliquot of the binding buffer was transferred to a new tube to quantitate the amount of unbound enzyme. Additional binding buffer was removed and the hair fragments were washed 4 times with 1 mL of 1% TWEEN®-20 in hydrolase buffer, followed by 2 washes with 1 mL each in hydrolase buffer. The hair fragments were then resuspended in 1 mL of hydrolase assay and the hydrolase activity that remained bound to the hair was measured. The C277S variant of Thermotoga maritime perhydrolase (SEQ ID NO: 293) was used as a control for an un-targeted perhydrolase. The results are provided in Table 2.

TABLE 2
Retention of Perhydrolase on Hair.

| | | | Activity |
|---|---|---|---|
| | | Activity in | retained on |
| | Activity^{a} | the first | hair after 4 |
| | unbound | TWEEN ®-20 | TWEEN ®-20 |
| Enzyme | (%) | wash (%) | washes (%) |
| Untargeted | 103 | 5 | 1 |
| T. maritima C277S | | | |
| (SEQ ID NO: 293) | | | |
| C277S-HC263 | 52 | 9 | 54 |
| (SEQ ID NO: 288) | | | |
| C277S-HC1010 | 20 | 20 | 41 |
| (SEQ ID NO: 289) | | | |

^{a}= The retention of perhydrolase on hair was detected by its hydrolase activity. 100% of activity is the hydrolase activity added to a tube containing ~20 mg of hair but not subjected to washes. For each enzyme, the 100% activity was: untargeted PAH, 148 μmol/min; C277S-HC263, 53 μmol/min; and C277S-HC1010, 125 μmol/min.

The data in Table 2 demonstrates that the perhydrolase fusions targeted to hair were retained on hair after extensive washes in 1% TWEEN®-20 whereas the untargeted perhydrolase was not.

### Example 3

### Construction and Production of Other Perhydrolases Targeted to Hair

The following example describes the design of expression systems for the production of additional perhydrolases targeted to hair. A summary of the constructs is provided in Table 3.

Briefly, the polynucleotide sequences (SEQ ID NOs: 9, 39, and 41) were designed to encode fusions of xylan esterases from Bacillus pumilus, Lactococcus lactis and Mesorhizobium loti (SEQ ID NOs 10, 40, and 42) to a 18 amino acid flexible linker (GPGSGGAGSPGSAGGPGS; SEQ ID NO: 285); itself fused to the hair-binding domains HC263 (SEQ ID NO 290). These enzymes belong to the CE-7 family of carbohydrate esterases as does the Thermotoga maritime perhydrolase.

The polynucleotide sequences (SEQ ID NOs: 322, 324, 326 and 328) were designed to encode fusions of the S54V variant of the aryl esterase from Mycobacterium smegmatis (SEQ ID NO: 314) to an 18 amino acid flexible linker (SEQ ID NO: 285); itself fused to the hair-binding domains HC263 (SEQ ID NO 290). The aryl esterase from Mycobacterium smegmatis belongs to a different class of hydrolytic enzyme than that of the Thermotoga maritime perhydrolase.

The polynucleotide sequences (SEQ ID NOs: 330, 332, 334, and 336) were designed to encode fusions of the L29P variant of the hydrolase from Pseudomonas fluorescens (SEQ ID NO: 315) to an 18 amino acid flexible linker (SEQ ID NO: 285); itself fused to the hair-binding domains HC263 (SEQ ID NO: 290). The esterase from Pseudomonas fluorescens belongs to a different class of hydrolytic enzymes than that of the Thermotoga maritime perhydrolase or of Mycobacterium smegmatis.

The genes were codon-optimized for expression in E. coli and synthesized by DNA2.0 (Menlo Park, Calif.). The coding sequences were cloned in plasmids behind the T7 promoter or the pBAD promoter in a manner similar as that described in Example 1. The plasmids were transferred in an appropriate expression host: E. coli strain BL21Al (Invitrogen, Carlsbad, Calif.) for constructs under the T7 promoter or in an AraBAD derivative of E. coli MG1655 for constructs under the pBAD promoter.

TABLE 3
Description of various hydrolase/perhydrolases fused
to targeting sequences with affinity for hair

| | | Nucleic Acid | Amino Acid |
|---|---|---|---|
| | | Sequence | sequence |
| | | Encoding the | of the |
| | Targeting | Targeted | Targeted |
| Organism source | Sequence | Perhydrolase | Perhydrolase |
| of perhydrolase | (SEQ ID NO:) | (SEQ ID NO:) | (SEQ ID NO:) |
| Bacillus | HC263 | 316 | 317 |
| pumilus | (SEQ ID NO: 290) | | |
| Lactococcus | HC263 | 318 | 319 |
| lactis | (SEQ ID NO: 290) | | |
| Mesorhizobium | HC263 | 320 | 321 |
| loti | (SEQ ID NO: 290) | | |
| Mycobacterium | HC263 | 322 | 323 |
| smegmatis | (SEQ ID NO: 290) | | |
| Mycobacterium | HC263KtoR | 324 | 325 |
| smegmatis | (SEQ ID NO: 312) | | |
| Mycobacterium | HC1010 | 326 | 327 |
| smegmatis | (SEQ ID NO: 291) | | |
| Mycobacterium | (GK)₅-His6 | 328 | 329 |
| smegmatis | (SEQ ID NO: 313) | | |
| Pseudomonas | HC263 | 330 | 331 |
| fluorescens | (SEQ ID NO: 290) | | |
| Pseudomonas | HC263KtoR | 332 | 333 |
| fluorescens | (SEQ ID NO: 312) | | |
| Pseudomonas | HC1010 | 334 | 335 |
| fluorescens | (SEQ ID NO: 291) | | |
| Pseudomonas | (GK)₅-His6 | 336 | 337 |
| fluorescens | (SEQ ID NO: 313) | | |

### Example 4

### Production of Fusion Proteins Comprising Alternative Esterase/Perhydrolase and a Hair-Binding Domain

This example describes the expression and purification of various alternative esterase/perhydrolase targeted to hair described in Example 3.

Strains expressing the genes encoding fusions to the perhydrolases in Table 3 of Example 3 were grown in 1 L of autoinduction medium (10 g/L Tryptone, 5 g/L Yeast Extract, 5 g/L NaCl, 50 mM Na₂HPO₄, 50 mM KH₂PO₄, 25 mM (NH₄)₂SO₄, 3 mM MgSO₄, 0.75% glycerol, 0.075% glucose and 0.05% arabinose) containing 50 mg/L spectinomycin at 37° C. for 20 hours under 200 rpm agitation. All protein fusions expressed well in E. coli. The cells were harvested by centrifugation at 8000 rpm at 4° C. and washed by resuspending the cell pellets in 300 mL of ice chilled lysis buffer (50 mM Tris, pH 7.5, 100 mM NaCl) using a tissue homogenizer (Brinkman Homogenizer model PCU11) at 3500 rpm followed by centrifugation (8000 rpm, 4° C.). The cells were disrupted by two passes through a French pressure cell at 16,000 psi (˜110.32 MPa). The lysed cell extracts were transferred to 4×50-mL conical polypropylene centrifuge tubes and centrifuged at 10,000 rpm for 10 min at 4° C. The supernatant containing the enzymes were transferred to new tubes. The approximate amount of fusion protein in each extract was estimated by comparison to bands of Bovine Serum Albumin standard on a Coomassie stained PAGE gel.

Since the perhydrolases fusions are not thermophilic, they were purified using their C-terminal His6 by metal chelation chromatography using Co-NTA agarose (HisPur Cobalt Resin, Thermo Scientific). Typically, cell extracts were loaded to a 5 to 10 mL column of Co-NTA agarose equilibrated with 4 volume of equilibration buffer (10 mM Tris HCl pH 7.5, 10% glycerol, 1 mM Imidazole and 150 mM NaCl). The amount of each extract loaded on the column was adjusted to contain between 5 and 10 mg of perhydrolase fusion per mL of Co-NTA agarose beads. The resin was washed with two bed volumes of equilibration buffer and was eluted with two volume of elution buffer (10 mM Tris HCl pH 7.5, 10% glycerol, 150 mM Imidazole, 500 mM NaCl). Fractions were collected and the presence of the purified proteins was detected by PAGE. The eluted proteins were analyzed by PAGE. All these proteins could be purified by affinity chromatography. That fact indicates that the fusion proteins were produced in the full length form.

This example demonstrates the feasibility of producing fusion hydrolases/perhydrolases from different families with a variety of binding domains having affinity to hair.

### Example 5

### Perhydrolase Activity of Alternative Perhydrolases Fused to a Hair-Binding Domains

The following example demonstrates the activity of alternative perhydrolases targeted to hair.

The perhydrolase activity of the enzymes targeted to hair with a variety of targeting domains produced as described in Examples 3 and 4 was measured with the ABTS assay. The results are reported in Table 4 and show that CE-7 (carbohydrate esterase family 7) as well as non-CE-7 hydrolases have perhydrolytic activity

TABLE 4
Perhydrolase Activity of Various Targeted Hydrolases.

| | | Targeted | |
|---|---|---|---|
| | | Perhydrolase | Specific |
| | Targeting | Amino Acid | perhydrolase |
| Organism source | Sequence | Sequence | activity |
| of perhydrolase | (SEQ ID NO:) | (SEQ ID NO:) | (μmol/mg/min) |
| Bacillus | HC263 | 317 | 40 |
| pumilus | (SEQ ID NO: 290) | | |
| Lactococcus | HC263 | 319 | 99 |
| lactis | (SEQ ID NO: 290) | | |
| Mesorhizobium | HC263 | 321 | 34 |
| loti | (SEQ ID NO: 290) | | |
| Mycobacterium | HC263 | 323 | 270 |
| smegmatis | (SEQ ID NO: 290) | | |
| Mycobacterium | HC263KtoR | 325 | 46 |
| smegmatis | (SEQ ID NO: 312) | | |
| Mycobacterium | HC1010 | 327 | 20 |
| smegmatis | (SEQ ID NO: 291) | | |
| Mycobacterium | (GK)₅-His6 | 329 | 264 |
| smegmatis | (SEQ ID NO: 313) | | |
| Pseudomonas | HC263 | 331 | 0.37 |
| fluorescens | (SEQ ID NO: 290) | | |
| Pseudomonas | HC263KtoR | 333 | 1.45 |
| fluorescens | (SEQ ID NO: 312) | | |
| Pseudomonas | HC1010 | 335 | 1.5 |
| fluorescens | (SEQ ID NO: 291) | | |
| Pseudomonas | (GK)₅-His6 | 337 | 2.65 |
| fluorescens | (SEQ ID NO: 313) | | |

Note:
The perhydrolase activity of the fusions of the Pseudomonas fluorescens hydrolase was assayed using 1M Na acetate at pH 5.5 instead of triacetin at pH 7.5 Targeted Perhydrolases HC1121 (C277S-HC263; SEQ ID NO: 288) had no detectable perhydrolase activity with acetate as a substrate.

This example demonstrates that other hair-targeting fusions of hydrolase enzymes, from the CE-7 family or from other families, show perhydrolytic activity and could be used directly or after enzyme evolution in hair applications.

### Example 6

### Hair Binding of Other Perhydrolases Targeted to Hair

The following example demonstrates that various targeted perhydrolases (other than the CE-7 Thermotoga maritime perhydrolase) can bind to hair.

Targeted Perhydrolases HC1121 (C277S-HC263; SEQ ID NO: 288), HC1169 (ArE-HC263; SEQ ID NO: 323) and variants of P. fluorescens perhydrolase (SEQ ID NO:331) were diluted to 50 μg/mL in a solution of 5% PEG-80 sorbitan laurate in 100 mM citrate-phosphate buffer adjusted to pH 6.0. Ten mg of human hair was added to 2 mL of the above formulations and incubated with gentle agitation for 5 minutes at room temperature to allow enzyme binding to hair. A no-enzyme control sample was also included. After binding, the binding solution was removed by aspiration and the hair was washed with 2 mL of 1% TWEEN®-20 in 50 mM pH 7.2 potassium phosphate buffer. The hair was removed from the tube, blotted dry with paper towel, and transferred to a new set of tubes. The hair was rinsed two times with 1% TWEEN®-20 in 50 mM pH 7.2 potassium phosphate buffer and then rinsed twice with 50 mM pH 7.2 potassium phosphate buffer. The amount of enzyme remaining bound to the hair was determined by SDS-PAGE analysis by cutting the hair into 3 mm fragments. The fragments were placed into a 500 μL polypropylene microcentrifuge tube and covered with 80 μL of gel loading buffer (20 μL NuPAGE LDS sample buffer (Invitrogen NP0007), 8 μL of 500 mM DTT, and 52 μL 50 mM pH 7.2 potassium phosphate). The hair samples were heated to 90° C. for 10 minutes, then cooled to 4 degrees.

The supernatant (25 μL) was loaded onto a NuPAGE 4-12% Bis-tris polyacrylamide gel (Invitrogen NP0322) and run at 150 v for 40 min. The gel was washed 3 times with water and stained in 15 mL SIMPLYBLUE™ Safestain (Invitrogen, Carlsbad, Calif.; LC6060) for 1 hour, rinsed 3 times, and then destained for 3 hours in water. The results are reported as relative intensity of enzyme band on the gel and provided in Table 5.

TABLE 5
Relative Binding of Various Perhydrolase Fusions on Hair.

| | | Targeted | Relative |
|---|---|---|---|
| Organism | Targeting | Perhydrolase | intensity |
| source of | sequence | Sequence | band on |
| perhydrolase | (SEQ ID NO:) | (SEQ ID NO) | PAGE |
| Thermotoga | HC263 | 288 | +++ |
| maritima | (SEQ ID NO: 290) | | |
| Mycobacterium | HC263 | 323 | +++ |
| smegmatis | (SEQ ID NO: 290) | | |
| Mycobacterium | HC263KtoR | 325 | +++ |
| smegmatis | (SEQ ID NO: 312) | | |
| Mycobacterium | HC1010 | 327 | + |
| smegmatis | (SEQ ID NO: 291) | | |
| Mycobacterium | (GK)₅-His6 | 329 | +++ |
| smegmatis | (SEQ ID NO: 313) | | |
| Pseudomonas | HC263 | 331 | +++ |
| fluorescens | (SEQ ID NO: 290) | | |
| Pseudomonas | HC263KtoR | 333 | ++ |
| fluorescens | (SEQ ID NO: 312) | | |
| Pseudomonas | HC1010 | 335 | + |
| fluorescens | (SEQ ID NO: 291) | | |
| Pseudomonas | (GK)₅-His6 | 337 | ++ |
| fluorescens | (SEQ ID NO: 313) | | |

The data indicates that diverse perhydrolases from different hydrolase families can be targeted to hair and that hair binding sequences are functional in the context of fusions to perhydrolases other than the Thermotoga perhydrolase.

### Example 7

### Preparation of Percarbonate/Triacetin Suspension as Substrate Stock for Perhydrolase to Generate Peracetic Acid (PAA)

The purpose of this example is to demonstrate that percarbonate and triacetin can be stored together in a non-aqueous environment as co-formulated substrate stock. Sodium percarbonate (Na₂CO₃.1.5 H₂O₂, MW 157.01; Sigma-Aldrich, St. Louis, Mo.) was white solid pellet, and was ground to powder using a mortar and pestle. As depicted in Table 6, different amounts of sodium percarbonate were weighed into glass vials followed by addition of triacetin and propylene glycol as solvent to make suspensions with 10 wt % solid which would supply substrates at desired concentration level when diluted with perhydrolase containing buffer. Stirring bars were added to the vials to keep stirring and percarbonate powder well suspended.

TABLE 6
Preparation of Sodium Percarbonate/Triacetin
as Co-formulated Substrate Stock.

| Substrate | | Equivalent | | |
|---|---|---|---|---|
| suspension | Triacetin | H₂O₂ | Percarbonate | Percarbonate |
| Stock ID | (mM) | (mM) | (mM) | wt % |
| 291-41-1 | 250 | 250 | 166.7 | 10 |
| 291-41-2 | 250 | 500 | 333.3 | 10 |
| 291-41-3 | 500 | 250 | 166.7 | 10 |
| 291-41-4 | 500 | 500 | 333.3 | 10 |

TABLE 7
Peracetic Acid Generation using Percarbonate/Triacetin Suspension Stocks.

| | | | pH 6.6, 50 | | | | Peracetic | Peracetic |
|---|---|---|---|---|---|---|---|---|
| | | | mM | Stock | HC1121 | | acid @ | acid @ |
| | | | Phosphate | suspension | 1 mg/mL | | 60 min | 60 min |
| Sample | Triacetin | H₂O₂ | Buffer | volume | stock | pH @ | day 1 | day 6 |
| ID | (mM) | (mM) | (μL) | (μL) | (μL) | 60 min | (ppm) | (ppm) |
| 291-41-1 | 250 | 250 | 764 | 226 | 10 | 9.2 | 5563 | 3915 |
| 291-41-2 | 250 | 500 | 528 | 462 | 10 | 9.9 | 7427 | 6151 |
| 291-41-3 | 500 | 250 | 773 | 217 | 10 | 8.4 | 6635 | 5291 |
| 291-41-4 | 500 | 500 | 537 | 453 | 10 | 9.5 | 11264 | 9557 |

After the substrate suspension stocks were made, proper volume of the well-mixed suspension stock was mixed with pH 6.6, 50 mM phosphate buffer, and 1 mg/mL HC1121 (C277S-linker-HC263; SEQ ID NO: 288) stock as depicted in Table 7, which made 1 mL reaction mixture with 10 μg/mL HC1121 working concentration, and planned substrate working concentrations (250 mM or 500 mM for triacetin, and 250 mM or 500 mM released H₂O₂). After the reaction proceeded for 60 min, the pH of the reaction mixture was measured and then the reaction was quenched by taking out 100 μL of liquid sample and adding into 900 μL 5 mM H₃PO₄. The quenched samples were filtered using a NANOSEP® MF centrifugal device (300K Molecular Weight Cutoff (MWCO), Pall Life Sciences, Ann Arbor, Mich.) by centrifugation for 6 min at 12,000 rpm. The filtrates were assayed by HPLC Karst assay in duplicates to determine the amount of peracetic acid (PAA) generated at those reaction conditions. The tests were run 1 day and 6 days after the suspension stocks were prepared, and the results of PAA generation at 60 min reaction time on both days are provided Table 7. The results show that PAA was generated in 60 min with percarbonate as a peroxygen source. After 6 days of storage, these substrate suspension stocks were still able to generate ca. 4000 to 9600 ppm PAA at 60 min reaction time, showing 70-85% of the PAA generation activity measured on Day 1.

Reference samples were run with identical concentration of liquid H₂O₂ in the same sample compositions as percarbonate samples as shown in Table 8, but only about half amount of PAA was generated after 60 min reaction (ca. 2700 ppm-4000 ppm). The pH for the liquid H₂O₂ samples was dominated by the 50 mM phosphate buffer, and the pH measured after 60 min reaction time ranged between pH 5.2 and pH 5.5. The pH for sodium percarbonate samples was dominated by the released sodium carbonate upon mixing with aqueous solutions, and the pH measured after 60 min reaction time ranged between pH 8.4 and pH 9.9. The perhydrolase HC1121 (SEQ ID NO: 288) used in this example had higher activity at higher pH as shown in Table 9.

TABLE 8
Peracetic Acid Generation using Liquid H₂O₂ and Triacetin.

| | | | 50 mM | | | | | | Peracetic |
|---|---|---|---|---|---|---|---|---|---|
| | | | Phosphate | Propylene | | HC1121 @ | 30% | | acid |
| Reference | Triacetin | H₂O₂ | Buffer | glycol | Triacetin | 1 mg/mL | H₂O₂ | pH @ | @ 60 min |
| Sample ID | (mM) | (mM) | (μL) | (μL) | (μL) | (μL) | (μL) | 60 min | (ppm) |
| Ref1 | 250 | 250 | 735 | 181 | 45 | 10 | 28.3 | 5.53 | 2780 |
| Ref2 | 250 | 500 | 471 | 416 | 45 | 10 | 56.7 | 5.25 | 2756 |
| Ref3 | 500 | 250 | 744 | 126 | 91 | 10 | 28.3 | 5.29 | 3131 |
| Ref4 | 500 | 500 | 480 | 362 | 91 | 10 | 56.7 | 5.17 | 3964 |

TABLE 9
PAA Generation at Different pH Using HC1121 and No Enzyme.

| | | | | reaction | |
|---|---|---|---|---|---|
| perhydrolase | triacetin | H2O2 | | time | PAA |
| conc | (mM) | (mM) | buffer | (min) | (ppm) |
| no enzyme | 100 | 250 | pH5, 50 mM | 5 | 51 |
| | | | citrate buffer | | |
| no enzyme | 100 | 250 | pH 5.6, 50 mM | 5 | 62 |
| | | | citrate buffer | | |
| no enzyme | 100 | 250 | pH 6, 50 mM | 5 | 49 |
| | | | citrate buffer | | |
| no enzyme | 100 | 250 | pH 6.6, 50 mM | 5 | 62 |
| | | | citrate- | | |
| | | | phosphate buffer | | |
| no enzyme | 100 | 250 | pH 7, 50 mM | 5 | 110 |
| | | | pyrophosphate | | |
| | | | buffer | | |
| 50 μg/mL | 100 | 250 | pH 5, 50 mM | 5 | 277 |
| HC1121 | | | citrate buffer | | |
| 50 μg/mL | 100 | 250 | pH 5.6, 50 mM | 5 | 1222 |
| HC1121 | | | citrate buffer | | |
| 50 μg/mL | 100 | 250 | pH 6, 50 mM | 5 | 2350 |
| HC1121 | | | citrate buffer | | |
| 50 μg/mL | 100 | 250 | pH 6.6, 50 mM | 5 | 4067 |
| HC1121 | | | citrate- | | |
| | | | phosphate buffer | | |
| 50 μg/mL | 100 | 250 | pH 7, 50 mM | 5 | 4832 |
| HC1121 | | | pyrophosphate | | |
| | | | buffer | | |

### Example 8

### Modulate PAA Generation from Percarbonate/Triacetin Suspension Stock

The purpose of this example is to demonstrate that the reaction pH and PAA generation level of the percarbonate/triacetin suspension stock could be modulated with proper buffer.

Three different buffers: (a) pH 6.6, 100 mM phosphate buffer, (b) pH 6.0, 100 mM phosphate buffer, and (3) pH 6.0, 200 mM phosphate buffer were used to make sodium percarbonate solutions at four different concentration levels (50 mM-200 mM equivalent H₂O₂ concentration). The pH of each solution was measured and is shown in Table 10. The pH 6.0, 200 mM phosphate buffer was able to modulate pH of percarbonate solutions at test concentration range to be between pH 6.5 and pH 8, a pH range deemed appropriate for personal care, particularly skin care products.

TABLE 10
pH for Percarbonate Solutions Made in Three Different Buffers

| | | | 100 mM, | 100 mM, | 200 mM, |
|---|---|---|---|---|---|
| | | Percar- | pH 6.6 | pH 6.0 | pH 6.0 |
| Percarbonate | Equivalent | bonate | buffer | buffer | buffer |
| solution ID | H₂O₂ (mM) | (mM) | (pH) | (pH) | (pH) |
| A | 200 | 133.3 | 9.7 | 9.5 | 7.6 |
| B | 150 | 100.0 | 9.5 | 8.9 | 7.3 |
| C | 100 | 66.7 | 8.4 | 7.6 | 7.0 |
| D | 50 | 33.3 | 7.3 | 7.1 | 6.7 |

To make sodium percarbonate/triacetin as co-formulated substrate stock, as depicted in Table 11, different amounts of sodium percarbonate powder were weighed into glass vials followed by addition of triacetin and propylene glycol as solvent if necessary to make suspensions with 5-10 wt % solid which would supply substrates at desired concentration level when diluted with perhydrolase containing buffer. Stirring bars were added to the vials to keep stirring and percarbonate powder well suspended.

TABLE 11
Preparation of Sodium Percarbonate/triacetin
as Co-formulated Substrate Stock

| Substrate | | | | Propylene | |
|---|---|---|---|---|---|
| suspension | Triacetin | H₂O₂ | Triacetin | glycol | Percarbonate |
| Stock ID | (mM) | (mM) | (μL) | (μL) | wt % |
| 291-42-7S | 250 | 50 | 2273 | 0 | 9 |
| 291-42-1S | 250 | 100 | 2273 | 1983 | 10 |
| 291-42-2S | 250 | 150 | 2273 | 4338 | 10 |
| 291-42-3S | 250 | 200 | 2273 | 6693 | 10 |
| 291-42-8S | 500 | 50 | 4546 | 0 | 5 |
| 291-42-4S | 500 | 100 | 4546 | 0 | 9 |
| 291-42-5S | 500 | 150 | 4546 | 1610 | 10 |
| 291-42-6S | 500 | 200 | 4546 | 3965 | 10 |

After the substrate suspension stocks were made, the proper volume of the well-mixed suspension stock was mixed with pH 6, 200 mM phosphate buffer, and 1 mg/mL HC1121 (SEQ ID NO: 288) stock or 1 mg/mL C277S stock (SEQ ID NO: 293) as shown in Table 12, which made 1 mL reaction mixture with 10 μg/mL HC1121 (SEQ ID NO: 288) or 10 μg/mL C277S (untargeted; SEQ ID NO: 293) working concentration, and the planned substrate working concentrations (ca. 250 mM or 500 mM for triacetin, and 50 mM-200 mM released H₂O₂). HC1121 (SEQ ID NO: 288) is a targeted perhydrolase comprising the C277S variant perhydrolase (SEQ ID NO: 293) coupled through a C-terminal 18 amino acid flexible peptide linker (SEQ ID NO: 285) to hair binding domain HC263 (SEQ ID NO: 290). C277S is the untargeted T. maritime variant perhydrolase (SEQ ID NO: 293). After the reaction proceeded for 60 min, the pH of the reaction mixture was measured. The reaction was quenched by taking out 100 μL of liquid sample and adding it into 900 μL of 100 mM H₃PO₄. The quenched samples were filtered using a NANOSEP® MF centrifugal device (300K Molecular Weight Cutoff (MWCO), Pall Life Sciences) by centrifugation for 6 min at 12,000 rpm. The filtrates were assayed by HPLC Karst assay in duplicates to determine the amount of peracetic acid (PAA) generated. Both the pH value and the amount of PAA generated after 60 min reaction time are provided in Table 12. A pH 6.7-pH 7.7 was observed for the pH 6.0, 200 mM phosphate buffered reaction mixtures, and ca. 1700 ppm-6000 ppm PAA was generated after 60 min depending upon substrate concentration. Increasing the substrate concentration increased the amount of PAA generated. Targeted perhydrolase HC1121 (SEQ ID NO: 288) and untargeted perhydrolase C277S (SEQ ID NO: 293) showed similar activity.

TABLE 12
Peracetic Acid Generation and pH After 60 min Reaction
Time using Percarbonate/Triacetin Suspension Stocks
and pH 6, 200 mM Phosphate Buffer with Targeted Perhydrolase
HC1121 or Untargeted Perhydrolase C277S

| | | Equivalent | Phosphate | Stock | | | Peracetic |
|---|---|---|---|---|---|---|---|
| | Triacetin | H₂O₂ | Buffer | suspension | | | acid |
| Sample ID | (mM) | (mM) | (μL) | (μL) | | pH | (ppm) |
| | | | | | HC1121 | | |
| | | | | | (μL)¹ | | |
| 291-42-7S | 250 | 50 | 945 | 45 | 10 | 6.7 | 1726 |
| 291-42-1S | 250 | 100 | 905 | 85 | 10 | 7.1 | 3025 |
| 291-42-2S | 250 | 150 | 858 | 132 | 10 | 7.4 | 3168 |
| 291-42-3S | 250 | 200 | 811 | 179 | 10 | 7.7 | 2871 |
| 291-42-8S | 500 | 50 | 899 | 91 | 10 | 6.7 | 2087 |
| 291-42-4S | 500 | 100 | 899 | 91 | 10 | 7.1 | 2836 |
| 291-42-5S | 500 | 150 | 867 | 123 | 10 | 7.4 | 4865 |
| 291-42-6S | 500 | 200 | 820 | 170 | 10 | 7.7 | 5987 |
| | | | | | C277S | | |
| | | | | | (μL)¹ | | |
| 291-42-7SB | 250 | 50 | 945 | 45 | 10 | 6.8 | 1831 |
| 291-42-8SB | 500 | 50 | 899 | 91 | 10 | 6.8 | 1989 |

¹= 1 mg/mL

### Example 9

### Hair Weakening and Bleaching (Lightening) Efficacy Using Perhydrolase with Percarbonate/Triacetin Suspension Stocks

The purpose of this example is to demonstrate hair weakening efficacy using the percarbonate/triacetin suspension stock with both targeted perhydrolase HC1121 (C2775-linker-HC263; SEQ ID NO: 288) and untargeted perhydrolase C277S (SEQ ID NO: 293).

Four substrate suspension stocks prepared in Example 8 were selected (291-42-1S; 291-42-4S; 291-42-7S; and 291-42-8S) and tested with both targeted perhydrolase HC1121 and untargeted perhydrolase C277S on hair samples with 24 hr treatment cycles. For each test condition, triplicates of hair tresses were used. The hair tresses were medium brown hair form International Hair Importers and Products (Glensdale, N.Y.). Each hair tress was glued at one end, and cut at 5 mm width and 4 cm long (excluding the glued portion), with 100+/−20 mg net hair weight. Each hair tress was placed in a clean plastic weighing tray (VWR, Cat. #12577-053). Each hair treatment solution was prepared, as shown in Table 13, by adding the proper volume of well-mixed percarbonate/triacetin suspension stock to a 3.5 mL 10 μg/mL enzyme solution prepared fresh each cycle from 5 mg/mL stock in pH 6.0, 200 mM phosphate buffer to achieve a 50 mM or 100 mM equivalent H₂O₂ working concentration, and a 250 mM or 500 mM triacetin working concentration. Then, 1 mL of the reaction mixture was added to each hair tress and rubbed into the hair tress with an applicator. The hair tress was sitting in this reaction mixture for 1 hr before being taken out to a dry dish. The hair tress was allowed to air dry for 23 hr and then was washed with 1 mL 1% sodium lauryl ether sulfate (SLES, RHODAPEX ES 2K″ by Rhodia Inc, Cranbury, N.J.) followed by a tap water rinse and paper towel dry. This completed a 24 hr treatment cycle. The treatment cycle was repeated 8-12 times depending upon a visual indication of hair damage.

Hair tresses became lighter-colored and weakened during the treatment. After final rinse and air-drying, L*, a*, b* color measurements were taken for each hair sample to quantify hair color loss, and L*, a*, b* color measurements were also taken for untreated hair as a reference for ΔE color difference calculations.

ΔE was calculated as

ΔE=((L*−L*_{ref})²+(a*−a*_{ref})²+(b*−b*_{ref})²)^{0.5}.

Tensile strength tests were conducted on each hair tress to quantify hair weakening as described above in General Methods.

At selected cycles of treatment the reaction mixture in which each hair tress was soaked was sampled (after the end of 1 hr soaking period) by taking 100 μL of reaction mixture and adding it to 900 μL 100 mM H₃PO₄ to quench the reaction. The quenched samples were filtered using a NANOSEP® MF centrifugal device (300K Molecular Weight Cutoff (MWCO), Pall Life Sciences) by centrifugation for 6 min at 12,000 rpm. The filtrates were assayed by HPLC Karst assay (supra) in duplicates to determine the amount of peracetic acid (PAA) generated. The PAA concentrations are summarized in Table 14. Compared to the no hair reference (control; PAA generation in 1 hr without hair was ca. 1700 ppm-3000 ppm), the PAA level in the reaction mixture after 1 hr hair treatment ranged from ca. 500 ppm-1800 ppm, indicating 40-80% of the PAA generated in 1 hr was apparently consumed during the hair treatment.

TABLE 13
Hair sample treatment solution preparation

| | | Enzyme solution | | | Equivalent | |
|---|---|---|---|---|---|---|
| Test | | 3.5 mL @ 10 | Substrate | Triacetin | H₂O₂ | Treatment |
| Condition | Hair sample ID | μg/mL | Suspension ID | (mM) | (mM) | cycles |
| 1 | 42-1 to 42-3 | HC1121 | 291-42-1S | 250 | 100 | 10 |
| 2 | 42-4 to 42-6 | HC1121 | 291-42-4S | 500 | 100 | 8 |
| 3 | 42-7 to 42-9 | HC1121 | 291-42-7S | 250 | 50 | 12 |
| 4 | 42-10 to 42-12 | HC1121 | 291-42-8S | 500 | 50 | 12 |
| 5 | 42-13 to 42-15 | C277S | 291-42-7S | 250 | 50 | 12 |
| 6 | 42-16 to 42-18 | C277S | 291-42-8S | 500 | 50 | 12 |

TABLE 14
Peracetic Acid Concentration After 1 hr Hair Treatment Using Percarbonate/Triacetin Suspension
Stocks with both Targeted Perhydrolase HC1121 and Untargeted Perhydrolase C277S

| Hair | Substrate | | | | PAA | PAA | PAA | PAA | PAA | PAA | PAA | PAA |
|---|---|---|---|---|---|---|---|---|---|---|---|---|
| Sample | Suspension | TA | H₂O₂ | | no hair | cycle 1 | cycle 2 | cycle 4 | cycle 6 | cycle 8 | cycle10 | cycle 12 |
| ID | ID | (mM) | (mM) | Enzyme | (ppm) | (ppm) | (ppm) | (ppm) | (ppm) | (ppm) | (ppm) | (ppm) |
| 42-13 to | 291-42-7S | 250 | 50 | C277S | 1831 | 524 | 460 | 771 | 468 | 562 | 492 | 567 |
| 42-15 | | | | | | | | | | | | |
| 42-16 to | 291-42-8S | 500 | 50 | C277S | 1989 | 754 | 661 | 824 | 694 | 617 | 683 | 804 |
| 42-18 | | | | | | | | | | | | |
| 42-7 to | 291-42-7S | 250 | 50 | HC1121 | 1726 | 442 | 478 | 649 | 408 | 462 | 1109 | 482 |
| 42-9 | | | | | | | | | | | | |
| 42-10 to | 291-42-8S | 500 | 50 | HC1121 | 2087 | 673 | 638 | 787 | 546 | 692 | 664 | 771 |
| 42-12 | | | | | | | | | | | | |
| 42-1 to | 291-42-1S | 250 | 100 | HC1121 | 3025 | 647 | 611 | 904 | 548 | 702 | 781 | |
| 42-3 | | | | | | | | | | | | |
| 42-4 to | 291-42-4S | 500 | 100 | HC1121 | 2836 | 904 | 981 | 1380 | 1556 | 1848 | | |
| 42-6 | | | | | | | | | | | | |

TABLE 15
Hair Tensile Strength and Hair Color Loss Results

| | | | | | | | | | hair color loss | |
|---|---|---|---|---|---|---|---|---|---|---|
| Hair | TA | H₂O₂ | | | Hair Tensile Strength, N/mgH | | | | avg. | stdev |
| Sample ID | (mM) | (mM) | Enzyme | Cycles | replicate1 | replicate2 | replicate3 | average | Δ E | Δ E |
| control | — | — | — | NAIR ® | 1.5 | — | — | — | — | — |
| | | | | 5 min | | | | | | |
| 42-13, 14, 15 | 250 | 50 | C277S | 12 | 0.64 | 0.48 | 0.38 | 0.50 | 17 | 2.0 |
| 42-16, 17, 18 | 500 | 50 | C277S | 12 | 0.69 | 0.19 | 0.23 | 0.37 | 18 | 4.2 |
| 42-7, 8, 9 | 250 | 50 | HC1121 | 12 | 0.17 | 0.31 | 0.27 | 0.25 | 20 | 0.8 |
| 42-10, 11, 12 | 500 | 50 | HC1121 | 12 | 0.25 | 0.18 | 0.20 | 0.21 | 21 | 1.0 |
| 42-1, 2, 3 | 250 | 100 | HC1121 | 10 | 0.04 | 0.05 | 0.34 | 0.14 | 21 | 1.2 |
| 42-4, 5, 6 | 500 | 100 | HC1121 | 8 | 0.00 | 0.00 | 0.05 | 0.02 | 27 | 3.0 |

The results in Table 15 indicated all treated hair was weakened significantly to below 0.5 N/mg hair tensile strength, far below the NAIR® 5 min treatment benchmark of 1.5 N/mg hair tensile strength. The higher the substrate concentration, the stronger weakening effect and the larger hair color loss. At the same substrate concentration level, targeted perhydrolase HC1121 (SEQ ID NO: 288) showed stronger hair weakening and hair lightening efficacy, even though similar level of PAA generation was detected for both enzymes (Table 12 and Table 14).

### Example 10

### Two-Compartment Depilatory Product Using Percarbonate/Triacetin Suspension Stock and Buffered Perhydrolase Stock

The purpose of this example is to demonstrate depilatory efficacy of a two-compartment product prototype with percarbonate/triacetin suspension stock on one compartment and buffered perhydrolase stock in the second compartment.

Similar to Example 8, sodium percarbonate/triacetin suspension as co-formulated substrate stock was prepared following the recipe in Table 16: sodium percarbonate powder was weighed into glass vials followed by addition of triacetin and propylene glycol as solvent to make suspensions with 5 wt % solid which would supply substrates at 250 mM triacetin and 100 mM H₂O₂ when diluted with perhydrolase containing buffer. Stirring bars were added to the vials to keep stirring and percarbonate powder well suspended.

Then 11 μg/mL solution of HC1121 was made by diluting the 5 mg/mL stock into pH 6, 200 mM phosphate buffer. The HC1121 solution was used as buffered perhydrolase stock. Each day, 1819 μL of this perhydrolase stock was mixed with 181 μL of the well-mixed percarbonate/triacetin suspension stock to make a 2-mL reaction mixture. Then, 0.5 mL of the 2-mL reaction mixture was transferred to one of the hair tress triplicates and was rubbed into the hair with an applicator. The hair tresses were medium brown hair form International Hair Importers. Each hair tress was glued at one end, and cut at 5 mm width and 4 cm long (excluding the glued portion), with 100+/−20 mg net hair weight. The hair was air dried for 24 hr before being washed with 1 mL 1% SLES followed by tap water rinse and paper towel dry. This treatment cycle was repeated for 14 cycles on each hair tress before measuring tensile strength test and conducting color measurement. The same test was carried out using an enzyme-free control where 1819 μL pH 6, 200 mM phosphate buffer (used in place of perhydrolase stock) was mixed with 181 μL of the percarbonate/triacetin suspension. The reaction conditions, the tensile test results and hair color loss results are summarized in Table 17. The enzyme free control lightened hair to similar degree as the HC1121 containing sample when using percarbonate/triacetin suspension as substrate stock, but didn't weaken hair as much. Targeted perhydrolase HC1121 at 10 μg/mL (working concentration) weakened the hair to the tensile strength at about 0.6 N/mg hair, much less than 1.5 N/mg NAIR® treated hair benchmark.

TABLE 16
Preparation of Sodium Percarbonate/Triacetin
as Co-formulated Substrate Stock.

| | Target working conc. | | Stock prep quantity | | | |
|---|---|---|---|---|---|---|
| | Triac- | | Triac- | Propylene | percar- | total |
| Substrate | etin | H₂O₂ | etin | glycol | bonate | vol. |
| Stock ID | (mM) | (mM) | (μL) | (μL) | wt % | (mL) |
| 291-44-S1 | 250 | 50 | 9092 | 8977 | 5 | 18 |

TABLE 17
Hair Treatment Conditions with Hair Tensile Strength and Hair Color Loss Results.

| | Reaction Condition | | | | | | | | |
|---|---|---|---|---|---|---|---|---|---|
| | HC1121 | | Equivalent | | | | | | |
| | working | TA | H₂O₂ | Hair Tensile Strength | | | | Hair color loss | |
| Reaction | conc. | working | working | (N/mgH) | | | | avg. | stdev |
| ID | (μg/mL) | conc (mM) | conc (mM) | replicate1 | replicate2 | replicate3 | average | Δ E | Δ E |
| 44-S1 | 0 | 250 | 50 | 2.55 | 2.05 | 2.33 | 2.31 | 14 | 0.3 |
| (control) | | | | | | | | | |
| 44-S1 | 10 | 250 | 50 | 0.40 | 0.63 | 0.72 | 0.58 | 16 | 2.8 |
| HC1121 | | | | | | | | | |

What is claimed is:

1. A hair care product comprising:
a) a non-aqueous composition comprising a mixture of:
1) at least one substrate selected from the group consisting of:
i) esters having the structure
[X]ₘR₅
wherein X=an ester group of the formula R₆C(O)O
R₆=C1 to C7 linear, branched or cyclic hydrocarbyl moiety, optionally substituted with hydroxyl groups or C1 to C4 alkoxy groups, wherein R₆ optionally comprises one or more ether linkages for R₆=C2 to C7;
R₅=a C1 to C6 linear, branched, or cyclic hydrocarbyl moiety or a five-membered cyclic heteroaromatic moiety or six-membered cyclic aromatic or heteroaromatic moiety optionally substituted with hydroxyl groups; wherein each carbon atom in R₅ individually comprises no more than one hydroxyl group or no more than one ester group or carboxylic acid group; wherein R₅ optionally comprises one or more ether linkages;
m is an integer ranging from 1 to the number of carbon atoms in R₅; and
wherein said esters have a solubility in water of at least 5 ppm at 25° C.;
ii) glycerides having the structure 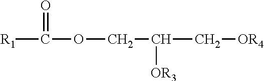
wherein R₁=C1 to C7 straight chain or branched chain alkyl optionally substituted with an hydroxyl or a C1 to C4 alkoxy group and R₃ and R₄ are individually H or R₁C(O);
iii) one or more esters of the formula 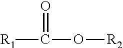
wherein R₁ is a C1 to C7 straight chain or branched chain alkyl optionally substituted with an hydroxyl or a C1 to C4 alkoxy group and R₂ is a C1 to C10 straight chain or branched chain alkyl, alkenyl, alkynyl, aryl, alkylaryl, alkylheteroaryl, heteroaryl, (CH₂CH₂O)ₙ, or (CH₂CH(CH₃)—O)ₙH and n is 1 to 10; and
iv) acetylated saccharides selected from the group consisting of acetylated monosaccharides, acetylated disaccharides, and acetylated polysaccharides; and
2) a solid source of peroxygen such as perborate, percarbonate or a combination thereof;
3) an optional organic cosolvent; and
b) an aqueous composition comprising
1) an enzyme catalyst having perhydrolytic activity;
2) at least one buffer; wherein the aqueous composition comprises a pH of at least 4; and
wherein the non-aqueous composition and the aqueous compositions remain separated prior to use and wherein an enzymatically generated peracid is produced upon combining the non-aqueous and aqueous compositions.

2. The hair care product of claim 1 wherein the buffer is selected from the group consisting of acetate, citrate, phosphate, pyrophosphate, glycine, bicarbonate, methylphosphonate, succinate, malate, fumarate, tartrate, maleate, and combinations thereof.

3. The hair care product of claim 1 wherein the enzyme having perhydrolytic activity is in the form of a fusion protein comprising:
a) a first portion comprising the enzyme having perhydrolytic activity; and
b) a second portion having a peptidic component having affinity for human hair.

4. The hair care product of claim 3 wherein the second portion is a single chain peptide comprising at least one hair-binding peptide.

5. The hair care product of claim 4 wherein the at least one hair-binding peptide range from 5 to 60 amino acids in length.

6. The hair care product of claim 3 wherein the hair care product is in the form of a multi-compartment packet, a multi-compartment bottle, at least two individual containers, and combinations thereof.

7. The hair care product of claim 1 wherein the non-aqueous composition and the aqueous composition are each substantially stable at 25° C. for at least 14 days.

8. The hair care product of claim 1 wherein the non-aqueous composition further comprises a desiccant.

9. The hair care product of claim 1 wherein the buffer in the aqueous composition is at a concentration of 10 mM to 1.0 M.

10. The hair care product of claim 1 further comprising a cosmetically acceptable carrier medium.

11. The hair care product of claim 3 wherein the enzyme catalyst having perhydrolytic activity comprises at least one enzyme having perhydrolytic activity selected from the group consisting of lipases, esterases, carbohydrate esterases, proteases, acyl transferases, aryl esterases, and combinations thereof.

12. The hair care product of claim 11 wherein the aryl esterase comprises an amino acid sequence having at least 95% identify to SEQ ID NO: 314.

13. The hair care product of claim 11 wherein the enzyme having perhydrolytic activity comprises an amino acid sequence having at least 95% identity to any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 293, 297, 299, 301, 303, 305, 307, 309, 311, 314, 315, 338, and 339.

14. The hair care product of claim 11 wherein the carbohydrate esterases are CE-7 carbohydrate esterases having a CE-7 signature motif that aligns with a reference sequence SEQ ID NO: 2 using CLUSTALW, said signature motif comprising:
a) an RGQ motif at positions corresponding to positions 118-120 of SEQ ID NO:2;
b) a GXSQG motif at positions corresponding to positions 179-183 of SEQ ID NO:2; and
c) an HE motif at positions corresponding to positions 298-299 of SEQ ID NO:2.

15. The hair care produce of claim 3 wherein the fusion protein comprises the following general structure:
PAH-[L]_{y}-HSBD
or
HSBD-[L]_{y}-PAH
wherein
PAH is the enzyme having perhydrolytic activity;
HSBD is a peptidic component having affinity for hair;
L is a linker ranging from 1 to 100 amino acids in length; and
y is 0 or 1.

16. The hair care product of claim 15 wherein the peptidic component having affinity for hair is an antibody, an F_{ab} antibody fragment, a single chain variable fragment (scFv) antibody, a Camelidae antibody, a scaffold display protein or a single chain polypeptide lacking an immunoglobulin fold.

17. The hair care product of claim 16 wherein the peptidic component having affinity for hair comprises a K_{D} value or an MB₅₀ value of 10⁻⁵ M or less for human hair.

18. The hair care product of claim 16 wherein the single chain polypeptide lacking an immunoglobulin fold comprises 2 to 50 hair-binding peptides, wherein the hair-binding peptides are independently and optionally separated by a polypeptide spacer ranging from 1 to 100 amino acids in length.

19. The hair care product of claim 16 wherein the peptidic component having affinity for hair comprises a net positive charge.

20. The hair care product of claim 1 wherein the organic cosolvent is selected from the group consisting of propylene glycol, dipropylene glycol, triethylene glycol, 1,3-propanediol, 1,3-butanediol, hexylene glycol, and any combination thereof.

21. A method to provide a peracid-based benefit to hair comprising
a) providing the hair care product of claim 1 or claim 3;
b) contacting hair with the enzymatically generated peracid produced when the aqueous composition and the non-aqueous composition are combined; whereby the hair receives a peracid-based benefit selected from the group consisting of hair removal, hair weakening, hair bleaching, hair styling, hair curling, hair conditioning, hair pretreating prior to application of a non-peracid-based benefit agent, and combinations thereof.

22. The method of claim 21 wherein the non-peracid-based benefit agent is a depilatory agent, a hair dye, a hair conditioning agent, and combinations thereof.

23. The method of claim 22 wherein an effective amount of peracid is generated, said effective amount ranging from 0.001 wt % to 4 wt %.

24. The method of claim 23 wherein the peracid is peracetic acid.

25. The method of claim 21 wherein the non-aqueous composition and the aqueous composition are combined prior to contacting human hair.

26. The method of claim 21 wherein the non-aqueous composition and the aqueous composition are applied simultaneously to human hair.

27. The method of claim 19 wherein the non-aqueous composition and the aqueous composition are applied sequentially to human hair.

28. The method of claim 27 wherein the non-aqueous composition is applied to human hair and then the aqueous composition is applied to the human hair.

29. The method of claim 27 wherein the aqueous composition is applied to human hair and then the non-aqueous composition is applied to the human hair.
